(19) United States
(12) Patent Application Publication
(10) Pub. No.: US 2008/0139546 A1
(43) Pub. Date: Jun. 12, 2008

(54) Tetrahydroquinoline, indoline, and related aniline derivatives of heterocycle-fused benzodioxan methylamines

(75) Inventors: Minsheng Zhang, Warren, NJ (US); Charles J. Stanton, Jersey City, NJ (US); Boyd Lynn Harrison, Princeton Junction, NJ (US); Terrance H. Andree, Doylestown, PA (US)

Correspondence Address: WilmerHale/Wyeth, 60 STATE STREET, BOSTON, MA, 02109, US

(73) Assignee: Wyeth, Madison, NJ (US)

(21) Appl. No.: 11/904,773

(22) Filed: Sep. 28, 2007

Related U.S. Application Data
(60) Provisional application No. 60/848,277, filed on Sep. 29, 2006.

(51) Int. Cl. A61K 31/4741; C07D 413/14; C07D 405/14; A61P 25/00; A61P 25/30; C12N 5/06; C12N 5/08; A61P 25/18; A61P 25/28; C07D 405/12; A61K 31/538

(52) U.S. Cl. 514/230.5; 546/90; 544/105; 514/291

(57) ABSTRACT

The present invention relates to a compound of the formula:

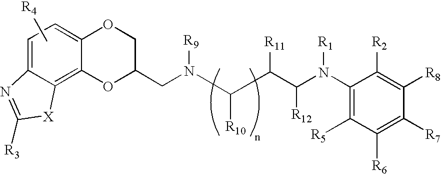

or an enantiomer, diastereomer, tautomer, or pharmaceutically acceptable salt or solvate thereof, useful as modulators of 5-HT_{1A} receptor activity and/or serotonin reuptake. These compounds are useful in treating nervous system disorders, such as anxiety-related disorders, cognition-related disorders, depression, schizophrenia, or sexual dysfunction and related illnesses.

### CROSS REFERENCE TO RELATED APPLICATION

This application claims the benefit of priority under 35 U.S.C. §119(e) to U.S. Patent Application Ser. No. 60/848,277 filed on Sep. 29, 2006, and is hereby incorporated by reference in its entirety.

### FIELD OF THE INVENTION

The invention relates to the preparation and use of novel tetrahydroquinoline, indoline, and related aniline derivatives of heterocycle-fused benzodioxan methylamine compounds.

### BACKGROUND

Depression is a serious health problem affecting a significant proportion of the population. Selective serotonin reuptake inhibitors (SSRIs) have produced success in treating depression and related illnesses and have become among the most prescribed drugs. They nonetheless have a slow onset of action, often taking several weeks to produce their full therapeutic effect. They also exhibit undesirable side effects, such as sexual dysfunction. Furthermore, SSRIs are effective in less than two-thirds of patients.

A number of serotonin (5-HT) receptors have been identified. It has been proposed that an antagonist of one such receptor, the 5-HT_{1A} receptor (also termed “5-HT_{1A}”), can improve the efficacy of the serotonin reuptake mechanism by blocking inhibitory 5-HT_{1A} autoreceptors (Perez et al., The Lancet, 349:1594-1597 (1997)). Likewise, 5-HT_{1A} agonists, such as gepirone, could have antidepressant-like activity by acting at postsynaptic 5-HT_{1A} receptors (Robinson et al., Clinical Therap. 25(6):1618-33 (2003)), and/or by producing a faster desensitization of 5-HT_{1A} autoreceptors. Such a combination therapy (5-HT_{1A} antagonist or 5-HT_{1A} agonist) would be expected to speed up the effect of the serotonin reuptake inhibitor. There is also evidence that 5-HT_{1A} partial agonism, combined with serotonin reuptake inhibition, can produce antidepressant-like effects (Hughes et al. Eur. J. Pharmacol, 510:49-57 (2005)). For example, vilazodone, a prototype SSRI/5-HT_{1A} partial agonist is currently in clinical trials for depression (de Paulis, Idrugs 10:193-201 (2007)).

Certain indole alkyl derivatives of heterocycle-fused benzodioxan methylamines have been reported to possess pharmaceutical activity (see, e.g., U.S. Pat. No. 6,800,637). In particular, these compounds are reported to have the ability to block the reuptake of serotonin, as well as having high affinity for 5-HT_{1A} serotonin receptors.

### SUMMARY

The invention relates to the synthesis and identification of compounds (5-HT_{1A} binding compounds) that are useful for modulation of serotonin (5-HT).

In one aspect, the invention provides a compound of Formula I, or an enantiomer, diastereomer, tautomer, or pharmaceutically acceptable salt or solvate thereof,

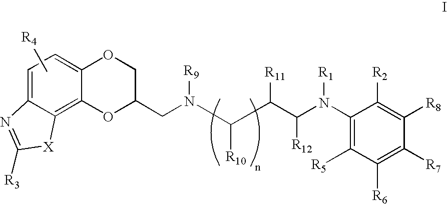

wherein
X is —O—, —N═CH—, —CR₁₃═CH—, —CR₁₃═N—, —CH═N—, —CH═CR₁₃—, —N═CR₁₃— or —NR₁₃—, in which R₁₃ is hydrogen or (C₁-C₆)-alkyl;

R₁ is hydrogen, (C₁-C₆)-alkyl, (C₁-C₆)-haloalkyl, (C₂-C₆)-alkenyl, or (C₂-C₆)-alkynyl;

R₂ is hydrogen, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₁₄, —OSO₂R₁₄, —SR₁₄, —SO₂R₁₄, —SO₂NR₁₄R₁₅, —NR₁₄R₁₅, —COR₁₄, —CO₂R₁₄, —NR₁₄CO₂R₁₅, —NR₁₄COR₁₅, —NR₁₄CONR₁₅, —NR₁₄SO₂R₁₅, or —CONR₁₄R₁₅; or

R₁ and R₂, when taken together with the atoms to which they are attached and the carbon interposed between them, form a 5- to 7-membered saturated ring, optionally containing an additional heteroatom selected from O, NR₁₄ or SOₘ;

R₃ is hydrogen, (C₁-C₆)-alkyl, halogen, or NR₁₄R₁₅;

R₄ is hydrogen, (C₁-C₆)-alkyl, halogen, —CF₃, —CN, —OR₁₄, —SO₂R₁₄, —NR₁₄SO₂R₅, —NR₁₄R₁₅, —COR₁₄, —CO₂R₁₄, —NR₁₄COR₁₅, or —CONR₁₄R₁₅;

R₉ is hydrogen or (C₁-C₃)-alkyl;

R₅, R₆, R₇, R₈, R₁₀, R₁₁, and R₁₂ are each independently hydrogen, halogen, (C₁-C₃)-alkyl, (C₁-C₃)-haloalkyl, (C₂-C₃)-alkenyl, or (C₂-C₃)-alkynyl, —CN, —CF₃, —NO₂, —CN, —C(O)NR₁₄ or —OR₁₄;

R₁₄ and R₁₅ are each independently is hydrogen, (C₁-C₆)-alkyl, (C₁-C₆)-haloalkyl, (C₂-C₆)-alkenyl, or (C₂-C₆)-alkynyl; and

n and m are each independently 0, 1, or 2;

wherein when X is —O—, R₁ and R₂ are taken together with the atoms to which they are attached and the carbon interposed between them to form a 5- to 7-membered saturated ring, optionally containing an additional heteroatom selected from O, NR₁₄ or SOₘ.

In some embodiments of this aspect, X is —O— or —CR₁₃═CH— and R₁₃ is hydrogen or (C₁-C₃)-alkyl. In some other embodiments, X is —CR₁₃═CH—. In some embodiments, X is —CR₁₃═CH— in which R₁₃ is hydrogen.

In some embodiments of this aspect, R₃ is hydrogen, —NH₂, or (C₁-C₆)-alkyl. In some other embodiments, R₃ is hydrogen or (C₁-C₃)-alkyl. In some embodiments, R₃ is methyl.

In some embodiments of this aspect, R₄ is hydrogen, halogen, —CN, —CF₃, (C₁-C₆)-alkyl, or OR₁₄, and R₁₄ is hydrogen or (C₁-C₆)-alkyl. In some embodiments, R₄ is hydrogen. In some other embodiments, R₁ is hydrogen or (C₁-C₆)-alkyl; R₂ is hydrogen, halogen, —CN, —CF₃, (C₁-C₆)-alkyl, or OR₁₄; and R₁₄ is hydrogen or (C₁-C₃)-alkyl. In some embodiments, R₁ is hydrogen or (C₁-C₃)-alkyl. In some other embodiments, R₂ is hydrogen, halogen or (C₁-C₃)-alkyl.

In some embodiments of this aspect, R₁ and R₂ are taken together with the atoms to which they are attached and the carbon interposed between them to form a 5- to 7-membered saturated ring, optionally containing an additional heteroatom selected from O, NR₁₄ or SOₘ. In some other embodiments, R₁ and R₂ are taken together with the atoms to which they are attached and the carbon interposed between them to form a 5- to 7-membered saturated ring optionally containing O.

In some embodiments of this aspect, R₅, R₆, R₇, and R₈ are each independently hydrogen, halogen, (C₁-C₃)-alkyl, —OR₁₄, —CN, or —C(O)NR₁₄ and R₁₄ is hydrogen or (C₁-C₆)-alkyl. In some other embodiments, R₅, R₆, R₇, and R₈ are each independently hydrogen, (C₁-C₃)-alkyl, or halogen. In some embodiments, R₅, R₆, R₇, and R₈ are each independently hydrogen or fluorine. In some other embodiments, one of R₅, R₆, R₇, and R₈ is hydrogen, (C₁-C₃)-alkyl, or halogen and the remaining R₅-R₈ are hydrogen. In some embodiments, two of R₅, R₆, R₇, and R₈ are each independently hydrogen, (C₁-C₃)-alkyl, —CN or halogen and the remaining R₅-R₈ are hydrogen.

In some embodiments of this aspect, one of R₅, R₆, and R₇ is hydrogen, (C₁-C₃)-alkyl, —CN or halogen and the remaining R₅-R₇ are each independently hydrogen and R₈ is hydrogen. In some other embodiments, two of R₅, R₆, and R₇ are each independently hydrogen, (C₁-C₃)-alkyl, —CN or halogen and the remaining R₅-R₇ is hydrogen and R₈ is hydrogen. In some embodiments, R₉ is methyl or ethyl. In some other embodiments, R₉ is hydrogen. In some embodiments, R₁₀, R₁₁, and R₁₂ are each independently hydrogen, (C₁-C₃)-alkyl, or halogen. In some other embodiments, R₁₀, R₁₁ and R₁₂ are each independently hydrogen. In some embodiments, n is 1 or 2.

In some embodiments of this aspect, X is —CR₁₃═CH— and R₁₃ is hydrogen or (C₁-C₃)-alkyl, R₁ is hydrogen or (C₁-C₆)-alkyl, R₂ is hydrogen, halogen, (C₁-C₆)-alkyl, —CN, —CF₃, or —OR₁; or R₁ and R₂, when taken together with the atoms to which they are attached and the carbon interposed between them, form a 5- to 7-membered unsaturated ring, optionally containing O; R₃ is hydrogen (C₁-C₆)-alkyl, or —NR₁₄, R₄ is hydrogen, halogen, (C₁-C₆)-alkyl, —CN, —CF₃, or —OR₁₄; R₅, R₆, R₇, and R₈ are each independently hydrogen, halogen, (C₁-C₃)-alkyl, —OR₁₄, —CN, or —C(O)NR₁₄; R₉ is hydrogen or (C₁-C₃)-alkyl; R₁₀, R₁₁ and R₁₂ are each independently hydrogen, halogen, or (C₁-C₆)-alkyl; R₁₄ is hydrogen or (C₁-C₆)-alkyl; and n is 1 or 2.

In some embodiments of this aspect, X is —CR₁₃═CH— in which R₁₃ is hydrogen, R₁ is hydrogen or (C₁-C₃)-alkyl; R₂ is hydrogen, (C₁-C₃)-alkyl, or halogen; or R₁ and R₂, when taken together with the atoms to which they are attached and the carbon interposed between them, form a 5- to 7-membered unsaturated ring, optionally containing O; R₃ is hydrogen or —CH₃; R₅, R₆, R₇ and R₈ are each independently hydrogen or halogen; R₉, R₁₀, R₁₁ and R₁₂ are each independently hydrogen or (C₁-C₃)-alkyl; and n is 1 or 2.

In some embodiments of this aspect, the invention provides a compound of Formula I having a structure of Formula Ib:

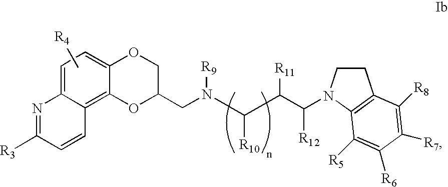

or an enantiomer, diastereomer, tautomer, pharmaceutically acceptable salt or solvate thereof, wherein R₃, R₄, R₅, R₆, R₇, R₈, R₁₀, R₁₁, R₁₂, and n are as defined hereinabove.

In some embodiments of this aspect, the invention provides a compound of Formula I having a structure of Formula Ic:

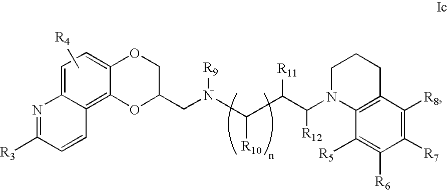

or an enantiomer, diastereomer, tautomer, pharmaceutically acceptable salt or solvate thereof, wherein R₃, R₄, R₅, R₆, R₇, R₈, R₁₀, R₁₁, R₁₂, and n are as defined hereinabove.

In some embodiments of this aspect, the invention provides a compound of Formula I having a structure of Formula Id:

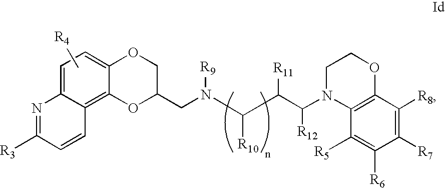

or an enantiomer, diastereomer, tautomer, pharmaceutically acceptable salt or solvate thereof, wherein R₃, R₄, R₅, R₆, R₇, R₈, R₁₀, R₁₁, R₁₂, and n are as defined hereinabove.

In some embodiments of this aspect, the invention provides a compound of Formula I having a structure of Formula Ie,

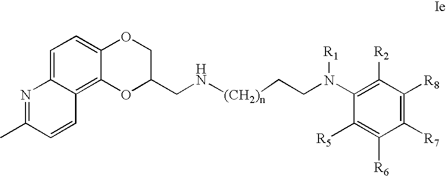

or an enantiomer, diastereomer, tautomer, or pharmaceutically acceptable salt or solvate thereof, wherein R₁, R₂, R₅, R₆, R₇, R₈, and n are as described hereinabove.

In some embodiments of this aspect, the compound of Formula I includes:
3-(3,4-dihydroquinolin-1(2H)-yl)-N-{[8-methyl-2,3-dihydro[1,4]dioxino[2,3-f]quinolin-2-yl]methyl}propan-1-amine;
3-(5-fluoro-2,3-dihydro-1H-indol-1-yl)-N-{[8-methyl-2,3-dihydro[1,4]dioxino[2,3-f]quinolin-2-yl]methyl}propan-1-amine;
3-(6-fluoro-2,3-dihydro-1H-indol-1-yl)-N-{[8-methyl-2,3-dihydro[1,4]dioxino[2,3-f]quinolin-2-yl]methyl}propan-1-amine;
3-(7-fluoro-3,4-dihydroquinolin-1(2H)-yl)-N-{[8-methyl-2,3-dihydro[1,4]dioxino[2,3-f]quinolin-2-yl]methyl}propan-1-amine;
3-(2,3-dihydro-1H-indol-1-yl)-N-{[8-methyl-2,3-dihydro[1,4]dioxino[2,3-f]quinolin-2-yl]methyl}propan-1-amine;
3-(2,3-dihydro-4H-1,4-benzoxazin-4-yl)-N-{[8-methyl-2,3-dihydro[1,4]dioxino[2,3-f]quinolin-2-yl]methyl}propan-1-amine;
3-(6,7-difluoro-3,4-dihydroquinolin-1(2H)-yl)-N-{[8-methyl-2,3-dihydro[1,4]dioxino[2,3-f]quinolin-2-yl]methyl}propan-1-amine;
3-(6-fluoro-3,4-dihydroquinolin-1(2H)-yl)-N-{[8-methyl-2,3-dihydro[1,4]dioxino[2,3-f]quinolin-2-yl]methyl}propan-1-amine;
3-(6,8-difluoro-3,4-dihydroquinolin-1(2H)-yl)-N-{[8-methyl-2,3-dihydro[1,4]dioxino[2,3-f]quinolin-2-yl]methyl}propan-1-amine;
3-(6-fluoro-2,3-dihydro-4H-1,4-benzoxazin-4-yl)-N-{[8-methyl-2,3-dihydro[1,4]dioxino[2,3-f]quinolin-2-yl]methyl}propan-1-amine;
N-{[8-methyl-2,3-dihydro[1,4]dioxino[2,3-f]quinolin-2-yl]methyl}-3-(2,3,4,5-tetrahydro-1H-1-benzazepin-1-yl)propan-1-amine;
N-(2-chlorophenyl)-N-methyl-N′-{[8-methyl-2,3-dihydro[1,4]dioxino[2,3-f]quinolin-2-yl]methyl}propane-1,3-diamine;
4-(6-fluoro-2,3-dihydro-1H-indol-1-yl)-N-{[8-methyl-2,3-dihydro[1,4]dioxino[2,3-f]quinolin-2-yl]methyl}butan-1-amine;
4-(3,4-dihydroquinolin-1(2H)-yl)-N-{[8-methyl-2,3-dihydro[1,4]dioxino[2,3-f]quinolin-2-yl]methyl}butan-1-amine;
4-(5-fluoro-2,3-dihydro-1H-indol-1-yl)-N-{[8-methyl-2,3-dihydro[1,4]dioxino[2,3-f]quinolin-2-yl]methyl}butan-1-amine; or

an enantiomer, diastereomer, tautomer, pharmaceutically acceptable salt or solvate thereof.

In some embodiments of this aspect, the compound of Formula I includes:
3-(3,4-dihydroquinolin-1(2H)-yl)-N-{[(2S)-8-methyl-2,3-dihydro[1,4]dioxino[2,3-f]quinolin-2-yl]methyl}propan-1-amine;
3-(5-fluoro-2,3-dihydro-1H-indol-1-yl)-N-{[(2S)-8-methyl-2,3-dihydro[1,4]dioxino[2,3-f]quinolin-2-yl]methyl}propan-1-amine;
3-(6-fluoro-2,3-dihydro-1H-indol-1-yl)-N-{[(2S)-8-methyl-2,3-dihydro[1,4]dioxino[2,3-f]quinolin-2-yl]methyl}propan-1-amine;
3-(7-fluoro-3,4-dihydroquinolin-1(2H)-yl)-N-{[(2S)-8-methyl-2,3-dihydro[1,4]dioxino[2,3-f]quinolin-2-yl]methyl}propan-1-amine;
3-(2,3-dihydro-1H-indol-1-yl)-N-{[(2S)-8-methyl-2,3-dihydro[1,4]dioxino[2,3-f]quinolin-2-yl]methyl}propan-1-amine;
3-(2,3-dihydro-4H-1,4-benzoxazin-4-yl)-N-{[(2S)-8-methyl-2,3-dihydro[1,4]dioxino[2,3-f]quinolin-2-yl]methyl}propan-1-amine;
3-(6,7-difluoro-3,4-dihydroquinolin-1(2H)-yl)-N-{[(2S)-8-methyl-2,3-dihydro[1,4]dioxino[2,3-f]quinolin-2-yl]methyl}propan-1-amine;
3-(6-fluoro-3,4-dihydroquinolin-1(2H)-yl)-N-{[(2S)-8-methyl-2,3-dihydro[1,4]dioxino[2,3-f]quinolin-2-yl]methyl}propan-1-amine;
3-(6,8-difluoro-3,4-dihydroquinolin-1(2H)-yl)-N-{[(2S)-8-methyl-2,3-dihydro[1,4]dioxino[2,3-f]quinolin-2-yl]methyl}propan-1-amine;
3-(6-fluoro-2,3-dihydro-4H-1,4-benzoxazin-4-yl)-N-{[(2S)-8-methyl-2,3-dihydro[1,4]dioxino[2,3-f]quinolin-2-yl]methyl}propan-1-amine;
N-{[(2S)-8-methyl-2,3-dihydro[1,4]dioxino[2,3-f]quinolin-2-yl]methyl}-3-(2,3,4,5-tetrahydro-1H-1-benzazepin-1-yl)propan-1-amine;
N-(2-chlorophenyl)-N-methyl-N′-{[(2S)-8-methyl-2,3-dihydro[1,4]dioxino[2,3-f]quinolin-2-yl]methyl}propane-1,3-diamine;
4-(6-fluoro-2,3-dihydro-1H-indol-1-yl)-N-{[(2S)-8-methyl-2,3-dihydro[1,4]dioxino[2,3-f]quinolin-2-yl]methyl}butan-1-amine;
4-(3,4-dihydroquinolin-1(2H)-yl)-N-{[(2S)-8-methyl-2,3-dihydro[1,4]dioxino[2,3-f]quinolin-2-yl]methyl}butan-1-amine;
4-(5-fluoro-2,3-dihydro-1H-indol-1-yl)-N-{[(2S)-8-methyl-2,3-dihydro[1,4]dioxino[2,3-f]quinolin-2-yl]methyl}butan-1-amine; or

an enantiomer, diastereomer, tautomer, pharmaceutically acceptable salt or solvate thereof.

In another aspect, the invention provides a compound of Formula Ie:

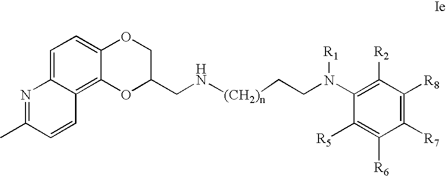

or an enantiomer, diastereomer, tautomer, pharmaceutically acceptable salt or solvate thereof, wherein

R₁ is hydrogen or (C₁-C₃)-alkyl;

R₂ is hydrogen, (C₁-C₃)-alkyl, or halogen; or

R₁ and R₂, when taken together with the atoms to which they are attached and the carbon interposed between them, form a 5- to 7-membered unsaturated ring, optionally containing O;

R₅, R₆, R₇, and R₈ are each independently hydrogen, —F or —Cl; and

n is 0, 1 or 2.

In some embodiments of this aspect, two of R₅, R₆, R₇, and R₈ are each independently hydrogen, —F or —Cl and the remaining R₅, R₆, R₇, or R₈ are each independently hydrogen.

In another aspect, compounds and pharmaceutically acceptable salts of the compounds of Formulae I, Ia, Ib, Ic, Id and le are also useful when formulated as pharmaceutical compositions. In some embodiments, these pharmaceutical compositions comprise compounds and pharmaceutically acceptable salts of the compounds of Formulae I, Ia, Ib, Ic, Id and Ie and a pharmaceutically acceptable carrier.

In another aspect, methods of synthesizing the compounds and pharmaceutically acceptable salts of compounds of Formulas I and Ie are provided.

In one aspect, a method of modulating 5-HT activity in a cell is provided. The method comprises providing a cell comprising a 5-HT_{1A} and contacting the cell with a compound of Formulae I, Ia, Ib, Ic, Id and Ie or a pharmaceutically acceptable salt thereof as described hereinabove in an amount and for a time sufficient for the compound to contact the 5-HT_{1A}. In certain embodiments of this aspect, the cell is a nervous system cell or a derivative thereof. In some embodiments, the 5-HT activity in the cell is increased. In some other embodiments, the 5-HT activity in the cell is decreased. In yet other embodiments, the 5-HT activity is increased and 5-HT transport is decreased.

In one aspect, a method of treating a 5-HT_{1A} related disorder in a subject is provided. The method comprises administering to the subject in need thereof an effective amount of a compound Formulae I, Ia, Ib, Ic, Id and Ie or a pharmaceutically acceptable salt thereof as described hereinabove.

In another aspect, a method of treating a central nervous system disorder in an animal is provided. The method comprises administering to the animal in need thereof an effective amount of a compound of Formulae I, Ia, Ib, Ic, Id and Ie or a pharmaceutically acceptable salt thereof as described hereinabove. In some cases of this aspect, the central nervous system disorder is an anxiety-related disorder, a cognition-related disorder, depression, or schizophrenia. In some other cases, the central nervous system disorder is a cognition-related disorder and the disorder is a cognitive deficit, dementia, Parkinson's disease, Huntington's disease, Alzheimer's disease, or schizophrenia. In some cases, the cognition-related disorder is a cognitive deficient and is cognitive deficit associated with Alzheimer's disease or mild cognitive impairment. In some other cases, the central nervous system disorder is an anxiety-related disorder and is attention deficit disorder, obsessive compulsive disorder, substance addiction, withdrawal from substance addiction, premenstrual dysphoric disorder, social anxiety disorder, anorexia nervosa, or bulimia nervosa.

In yet another aspect, a method of modulating the activity of a 5-HT_{1A} receptor is provided. The method comprises providing a 5-HT_{1A} receptor and contacting the receptor with at a compound of Formulae I, Ia, Ib, Ic, Id and Ie or a pharmaceutically acceptable salt thereof as described hereinabove in an amount and for a time sufficient to modulate 5-HT_{1A} activity. In certain embodiments of this aspect, the 5-HT_{1A} receptor is in a subject. In certain other embodiments, the 5-HT_{1A} receptor is in a human.

In yet another aspect, a method of modulating 5-HT_{1A}-mediated activity in a cell is provided. The method comprises providing a cell comprising 5-HT_{1A} and contacting the cell with a compound of Formulae I, Ia, Ib, Ic, Id and Ie or a pharmaceutically acceptable salt thereof as described hereinabove such that at least one 5-HT_{1A}-mediated activity is modulated. In certain embodiments of this aspect, the 5-HT_{1A}-mediated activity is cyclic AMP (cAMP) level and cAMP is increased compared to a control. In certain other embodiments, the cell is in a human.

In yet another aspect, a method of modulating serotonin reuptake in a cell is provided. The method comprises providing a cell comprising a serotonin transporter; and contacting the cell with a compound of Formulae I, Ia, Ib, Ic, Id and Ie or a pharmaceutically acceptable salt thereof as described hereinabove such that 5-HT reuptake is modulated. In certain embodiments of this aspect, the cell is in a human.

In yet another aspect, a pharmaceutical composition for treating a 5-HT_{1A} related disorder is provided. The composition comprises a compound of Formulae I, Ia, Ib, Ic, Id and Ie or a pharmaceutically acceptable salt thereof as described hereinabove.

In yet another aspect, a pharmaceutical composition for treating a central nervous system disorder is provided. The composition comprises a compound of Formulae I, Ia, Ib, Ic, Id and Ie or a pharmaceutically acceptable salt thereof as described hereinabove.

In a further aspect, the invention provides a use of a compound of Formulae I, Ia, Ib, Ic, Id and Ie or a pharmaceutically acceptable salt thereof as described hereinabove in the preparation of a medicament for the treatment of a 5-HT_{1A} related disorder, the treatment of a central nervous system disorder, the modulation of the activity of a 5-HT_{1A} receptor, the modulation of 5-HT_{1A}-mediated activity in a cell or the modulation of serotonin reuptake in a cell.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Other features and advantages of the invention will be apparent from the detailed description, drawings, and from the claims.

### DETAILED DESCRIPTION

The present invention relates to the identification of novel compounds that are useful as modulators of 5-HT_{1A} receptors and can block serotonin reuptake (e.g., as a serotonin reuptake inhibitor (SSRI)). Certain compounds have activity as 5-HT_{1A} agonists and some can act as of 5-HT_{1A} partial agonists. A partial agonist can display agonism or antagonism depending upon the local environment, levels of endogenous transmitter and sensitivity of the receptor, as well as depending upon the second messenger system through which it might work. Accordingly, certain compounds disclosed herein can display 5-HT antagonist activity.

Compounds described herein are useful for treating disorders that involve 5-HT_{1A} activity (5-HT_{1A} related disorders). For example, drugs that combine 5-HT transporter blockade and 5-HT_{1A} agonism can be useful for treating depression.

The term “(C₁-C₆)-alkyl” as used herein refers to a linear or branched, saturated hydrocarbon having from 1 to 6 carbon atoms. Representative (C₁-C₆)-alkyl groups include, but are not limited to methyl, ethyl, propyl, isopropyl, butyl, sec-butyl, tert-butyl, pentyl, isopentyl, neopentyl, hexyl, isohexyl, and neohexyl. In some embodiments, the (C₁-C₆)-alkyl group is optionally substituted with one or more, e.g. one to three, of the following groups (which may be the same or different): halogen, —N₃, —NO₂, —CN, —OR′, —SR′, —SO₂R′, —SO₂N(R′)₂, —N(R′)₂, —COR′, —CO₂R′, —NR′CO₂R′, —NR′COR′, —NR′CONR′, or —CON(R′)₂, wherein each R′ is independently hydrogen or unsubstituted (C₁-C₆)-alkyl. Similarly, “(C₁-C₃)-alkyl” as used herein refers to a linear or branched, saturated hydrocarbon, optionally substituted as described above, having from 1 to 3 carbon atoms. In certain embodiments some or all of the (C₁-C₆)-alkyl groups are (C₁-C₃)-alkyl groups.

The term “(C₂-C₆)-alkenyl” as used herein refers to a linear or branched hydrocarbon having from 2 to 6 carbon atoms and having at least one carbon-carbon double bond. In one embodiment, the (C₂-C₆)-alkenyl has one double bond. In another embodiment, the (C₂-C₆)-alkenyl has two double bonds. The (C₂-C₆)-alkenyl moiety may exist in the E or Z conformation and the compounds described herein include both conformations. In one embodiment, the (C₂-C₆)-alkenyl group is optionally substituted with one or more, e.g. one to three, of the following groups (which may be the same or different): halogen, —N₃, —NO₂, —CN, —OR′, —SR′, —SO₂R′, —SO₂N(R′)₂, —N(R′)₂, —COR′, —CO₂R′, —NR′CO₂R′, —NR′COR′, —NR′CONR′, or —CON(R′)₂, wherein each R′ is independently hydrogen or unsubstituted (C₁-C₆)-alkyl. Similarly, “(C₂-C₃)-alkenyl” and “C₃-alkenyl” as used herein refer to a linear or branched hydrocarbon, optionally substituted as described above, having from 2 to 3, or 3 carbon atoms, respectively, and having at least one carbon-carbon double bond. In certain embodiments some or all of the (C₂-C₆)-alkenyl groups are (C₂-C₃)-alkenyl. Examples of suitable alkenyl groups include ethenyl and allyl.

The term “(C₂-C₆)-alkynyl” as used herein refers to a linear or branched hydrocarbon having from 2 to 6 carbon atoms and having at least one carbon-carbon triple bond. In one embodiment, the (C₂-C₆)-alkenyl group is optionally substituted with one or more, e.g. one to three, of the following groups (which may be the same or different): halogen, —N₃, —NO₂, —CN, —OR′, —SR′, —SO₂R′, —SO₂N(R′)₂, —N(R′)₂, —COR′, —CO₂R′, —NR′CO₂R′, —NR′COR′, —NR′CONR′, or —CON(R′)₂, wherein each R′ is independently hydrogen or unsubstituted (C₁-C₆)-alkyl. Similarly, “(C₂-C₃)-alkynyl” and “C₃-alkynyl” as used herein refers to a linear or branched hydrocarbon, optionally substituted as described above, having from 2 to 3, or 3 carbon atoms, respectively, and having at least one carbon-carbon triple bond. In certain embodiments some or all of the (C₂-C₆)-alkynyl groups are (C₂-C₃)-alkynyl groups.

“(C₁-C₆)-haloalkyl” refers to a C₁-C₆ alkyl group, as defined herein, wherein one or more of the C₁-C₆ alkyl group's hydrogen atoms has been replaced with —F, —Cl, —Br or —I. Representative examples of an alkylhalo group include, but are not limited to, —CH₂F, —CCl₃, —CF₃, —CH₂Cl, —CH₂CH₂Br, —CH₂CH₂I, —CH₂CH₂CH₂F, —CH₂CH₂CH₂Cl, —CH₂CH₂CH₂CH₂Br, —CH₂CH₂CH₂CH₂I, —CH₂CH₂CH₂CH₂CH₂Br, —CH₂CH₂CH₂CH₂CH₂I, —CH₂CH(Br)CH₃, —CH₂CH(Cl)CH₂CH₃, —CH(F)CH₂CH₃, —C(CH₃)₂(CH₂Cl), —CH₂CH₂CH₂CH₂CH₂CH₂Br, and —CH₂CH₂CH₂CH₂CH₂CH₂I.

The term “administer”, “administering”, or “administration”, as used herein refers to either directly administering a compound or pharmaceutically acceptable salt of the compound or a composition to an animal, or administering a prodrug derivative or analog of the compound or pharmaceutically acceptable salt of the compound or composition to the animal, which can form an amount of the compound equivalent to an amount of the compound that can be directly administered or an amount of the prodrug derivative or analog that is pharmaceutically effective (“an equivalent amount”) of the compound within the animal's body.

The term “amine protecting group” as used herein refers to a moiety that temporarily blocks an amine reactive site in a compound. Generally, this is done so that a chemical reaction can be carried out at another reactive site in a multifunctional compound or to otherwise stabilize the amine. In one embodiment, an amine protecting group is selectively removable by a chemical reaction. An exemplary amine protecting group is an organoxycarbonyl group, i.e. where the amine is protected as a carbamate. Carbamates include, without limitation, t-butyl carbamate, methyl carbamate, ethyl carbamate, 2,2,2-trichloroethyl carbamate, 2-(trimethylsilyl)ethyl carbamate, 1,1-dimethyl-2,2,2-trichloroethyl carbamate, benzyl carbamate, p-methoxybenzyl carbamate, p-nitrobenzylcarbamate, p-bromobenzyl carbamate, p-chlorobenzyl carbamate, and 2,4-dichlorobenzyl carbamate. In one embodiment, the amine protecting group is t-BOC (t-butoxycarbonyl). In another embodiment, the amine protecting group is CBZ (benzyloxycarbonyl). For more exemplary amine protecting groups, see, e.g., Greene and Wuts, Protecting Groups in Organic Synthesis, Third Edition, John Wiley & Sons (1999).

The term “leaving group” as used herein refers to a moiety in a molecule which takes part in a nucleophilic substitution reaction. During the reaction, a nucleophile replaces the leaving group to produce a new molecule. Exemplary leaving groups include, but not limited to, halides; sulfonic ester groups such as tosylate, brosylate, nosylate, and mesylate; oxonium ions; alkyl perchlorates; ammonioalkanesulfonate esters (betylates); alkyl fluorosulfonates; and the fluorinated compounds triflates and nonaflates. See, Jerry March, Advanced Organic Chemistry, Fourth Edition, John Wiley & Sons, pages 352-357 (1992). In certain embodiments, the leaving group is a sulfonic ester. In certain other embodiments, the leaving group is a moiety of the structure

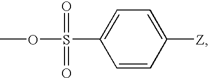

wherein Z is —NO₂, halogen, —CH₃ or —CF₃. In some cases, the leaving group is brosylate, tosylate, or mesylate.

The term “animal” as used herein includes, without limitation, a human, mouse, rat, guinea pig, dog, cat, horse, cow, pig, non-human primate (e.g., monkey, chimpanzee, baboon, or Rhesus Macaque). In one embodiment, the animal is a mammal. In another embodiment, the animal is a human.

The term “conditions effective to” as used herein refers to synthetic reaction conditions that will be apparent to those skilled in the art of synthetic organic chemistry.

The term “effective amount” as used herein refers to an amount of a compound or pharmaceutically acceptable salt of a compound that, when administered to an animal, is effective to prevent, to at least partially ameliorate, or to cure, a condition from which the animal suffers or is suspected to suffer.

The term “halogen” as used herein refers to fluorine, chlorine, bromine, and iodine.

The term “isolated” as used herein refers to separate from other components of a reaction mixture, or a natural source. In certain embodiments, the isolate contains at least about 50%, at least about 55%, at least about 60%, at least about 65%, at least about 70%, at least about 75%, at least about 80%, at least about 85%, at least about 90%, at least about 95%, or at least about 98% of the compound or pharmaceutically acceptable salt of the compound by weight of the isolate.

The term “pharmaceutically acceptable salt”, as used herein, refers to salts derived from organic and inorganic acids of a compound described herein. Exemplary salts include, but are not limited to, sulfate, citrate, acetate, oxalate, chloride, hydrochloride, bromide, hydrobromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, succinate, fumarate, maleate, malonate, mandelate, malate, phthalate, and pamoate. The term “pharmaceutically acceptable salt” as used herein also refers to a salt of a compound described herein having an acidic functional group, such as a carboxylic acid functional group, and a base. Exemplary bases include, but are not limited to, hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like. The term “pharmaceutically acceptable salt” also includes hydrates of a compound described herein.

The term “substantially free of its corresponding opposite enantiomer” as used herein means that the compound contains no more than about 10% by weight of its corresponding opposite enantiomer. In other embodiments, the compound that is substantially free of its corresponding opposite enantiomer contains no more than about 5%, no more than about 1%, no more than about 0.5%, or no more than about 0.1% by weight of its corresponding opposite enantiomer. An enantiomer that is substantially free of its corresponding opposite enantiomer includes a compound that has been isolated and purified or has been prepared substantially free of its corresponding opposite enantiomer.

When ranges are used herein for physical properties, such as molecular weight, or chemical properties, such as chemical formulae, all combinations and subcombinations of ranges of specific embodiments therein are intended to be included.

The disclosure of each patent, patent application, and publication cited or described in this document is hereby incorporated by reference, in their entirety.

In one embodiment, compounds of Formula I are provided:

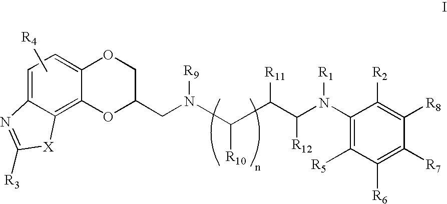

or an enantiomer, diastereomer, tautomer, pharmaceutically acceptable salt or solvate thereof, wherein:

X is —O—, —N═CH—, —CR₁₃═CH—, —CR₁₃═N—, —CH═N—, —CH═CR₁₃—, —N═CR₁₃— or —NR₁₃—, in which R₁₃ is hydrogen or (C₁-C₆)-alkyl;

R₁ is hydrogen, (C₁-C₆)-alkyl, (C₁-C₆)-haloalkyl, (C₂-C₆)-alkenyl, or (C₂-C₆)-alkynyl;

R₂ is hydrogen, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₁₄, —OSO₂R₁₄, —SR₁₄, —SO₂R₁₄, —SO₂NR₁₄R₁₅, —NR₁₄R₁₅, —C(O), —COR₁₄, —CO₂R₁₄, —NR₁₄CO₂R₁₅, —NR₁₄COR₁₅, —NR₁₄CON R₁₄R₁₅, —NR₁₄SO₂R₁₅, or —CONR₁₄R₁₅; or

R₁ and R₂, when taken together with the atoms to which they are attached and the carbon interposed between them, form a 5- to 7-membered saturated ring, optionally containing an additional heteroatom selected from O, NR₁₄ or SOₘ;

R₃ is hydrogen, (C₁-C₆)-alkyl, halogen, or —NR₁₄R₁₅;

R₄ is hydrogen, (C₁-C₆)-alkyl, halogen, —CF₃, —CN, —OR₁₄, —SO₂R₁₄, —NR₁₄SO₂R₁₅, —NR₁₄R₁₅, —COR₁₄, —CO₂R₁₄, —NR₁₄COR₁₅, or —CONR₁₄R₁₅;

R₉ is hydrogen or (C₁-C₃)-alkyl;

R₅, R₆, R₇, R₈, R₁₀, R₁₁ and R₁₂ are each independently hydrogen, halogen, (C₁-C₃)-alkyl, (C₁-C₃)-haloalkyl, (C₂-C₃)-alkenyl, (C₂-C₃)-alkynyl, —CN, —CF₃, —NO₂, —CN, —C(O)NR₁₄ or —OR₁₄;

R₁₄ and R₁₅ are each independently hydrogen, (C₁-C₆)-alkyl, (C₁-C₆)-haloalkyl, (C₂-C₆)-alkenyl, or (C₂-C₆)-alkynyl;

n and m are each independently 0, 1, or 2; and

wherein when X is —O—, R₁ and R₂ are taken together with the atoms to which they are attached and the carbon interposed between them, to form a 5- to 7-membered saturated ring, optionally containing an additional heteroatom selected from —O—, —NR₁₄ or —SOₘ—.

In some embodiments, X is —O— or —CR₁₃═CH— and R₁₃ is hydrogen or (C₁-C₃)-alkyl. In some embodiments, X is —CR₁₃═CH—. In some embodiments, X is —CR₁₃═CH— in which R₁₃ is hydrogen.

In some embodiments, R₃ is hydrogen, —NH₂, or (C₁-C₆)-alkyl. In some embodiments, R₃ is hydrogen or (C₁-C₃)-alkyl. In some embodiments, R₃ is methyl.

In some embodiments, X is —O— or —CR₁₃═CH—, R₃ is hydrogen, —NH₂, or (C₁-C₆)-alkyl, and R₁₃ is hydrogen or (C₁-C₃)-alkyl. In some embodiments, X is —CR₁₃═CH—, R₃ is hydrogen or (C₁-C₃)-alkyl, and R₁₃ is hydrogen or (C₁-C₃)-alkyl. In some embodiments, X is —CH═CH— and R₃ is methyl.

In some embodiments, R₄ is hydrogen, halogen, —CN, —CF₃, (C₁-C₆)-alkyl, or OR₁₄, and R₁₄ is hydrogen or (C₁-C₆)-alkyl. In some embodiments, R₄ is hydrogen, halogen, or OR₁₄, and R₁₄ is hydrogen or (C₁-C₃)-alkyl. In some embodiments, R₄ is hydrogen.

In some embodiments, R₁ is hydrogen or (C₁-C₃)-alkyl. In some embodiments, R₁ is methyl or ethyl.

In some embodiments, when R₁ is (C₂-C₆)-alkenyl or (C₂-C₆)-alkynyl, the carbon-carbon double or triple bond, respectively, is not bonded to the carbon immediately adjacent to the nitrogen atom. For example, in some embodiments, R₁ is —CH₂—CH═CH₂, —CH₂—CH═CH—CH₃, —CH₂—CH₂—CH═CH₂, —CH₂—CH═CH—CH₂—CH₃, —CH₂—CH₂—CH═CH—CH₃, —CH₂—CH₂—CH₂—CH═CH₂, —CH₂—CH═CH—CH₂—CH₂—CH₃, —CH₂—CH₂—CH═CH—CH₂—CH₃, —CH₂—CH₂—CH—CH═CH₂—CH₃, —CH₂—CH₂—CH₂—CH₂—CH═CH₂, —CH₂—C≡CH, —CH₂—C≡C—CH₃, —CH₂—CH₂—C≡CH, —CH₂—C≡C—CH₂—CH₃, —CH₂—CH₂—C≡C—CH₃, —CH₂—CH₂—CH₂—C≡CH, —CH₂—C≡C—CH₂—CH₂—CH₃, —CH₂—CH₂—C≡C—CH₂—CH₃, —CH₂—CH₂—CH₂—C≡C—CH₃, or —CH₂—CH₂—CH₂—CH₂—C≡CH.

In some embodiments, R₂ is hydrogen, halogen, —CN, —CF₃, (C₁-C₆)-alkyl, or OR₁₄, and R₁₄ is hydrogen or (C₁-C₃)-alkyl. In some embodiments, R₂ is hydrogen, halogen or (C₁-C₃)-alkyl. In some embodiments, R₂ is hydrogen, —Cl, methyl or ethyl. In some embodiments, R₂ is —Cl.

In some embodiments, R₁ and R₂, when taken together with the atoms to which they are attached and the carbon interposed between them, form a 5- to 7-membered saturated ring, optionally containing an additional heteroatom selected from O, NR₁₄ or SOₘ. In some embodiments, the heteroatom is located in the para position relative to the nitrogen in a 6-membered saturated ring formed from R₁ and R₂. In some embodiments, R₁ and R₂, taken together with the atoms to which they are attached and the carbon interposed between them, form a 5- to 7-membered saturated ring, optionally containing O. In some embodiments, R₁ and R₂, taken together with the atoms to which they are attached and the carbon interposed between them, form a 5- to 7-membered saturated alkyl ring. In some embodiments, R₁ and R₂, taken together with the atoms to which they are attached and the carbon interposed between them, form a 5- to 7-membered saturated heteroalkyl ring containing one oxygen atom. In some embodiments, the oxygen atom is located in the para position relative to the nitrogen in a 6-membered saturated heteroalkyl ring formed from R₁ and R₂.

In some embodiments, R₁ is hydrogen or (C₁-C₆)-alkyl. In some other embodiments, R₁ is hydrogen or (C₁-C₂)-alkyl.

In some embodiments, R₂ is hydrogen, —Cl, or (C₁-C₂)-alkyl.

In some embodiments, R₅, R₆, R₇, and R₈ are each independently hydrogen, halogen, (C₁-C₃)-alkyl, —OR₁₄, —CN, or —C(O)NR₁₄. In some embodiments, R₅, R₆, R₇, and R₈ are each independently hydrogen, halogen, (C₁-C₃)-alkyl, —OR₁₄, —CN, or —C(O)NR₁₄ and R₁₄ is hydrogen or (C₁-C₃)-alkyl. In some embodiments, R₅, R₆, R₇, and R₈ are each independently hydrogen, (C₁-C₃)-alkyl, —CN, or halogen. In some embodiments, R₅, R₆, R₇, and R₈ are each independently hydrogen, —Cl or —F. In some embodiments, R₅, R₆, R₇, and R₈ are each independently hydrogen or —F.

In some embodiments, one of R₅, R₆, R₇, and R₈ is hydrogen, (C₁-C₃)-alkyl, —CN, or halogen and the remaining R₅-R₈ are hydrogen. In some embodiments, one of R₅, R₆, R₇, and R₈ is hydrogen, —Cl or —F and the remaining R₅-R₈ are hydrogen. In some embodiments, one of R₅, R₆, R₇, and R₈ is hydrogen or —F and the remaining R₅-R₈ are hydrogen.

In some embodiments, two of R₅, R₆, R₇, and R₈ are hydrogen, (C₁-C₃)-alkyl, —CN or halogen and the remaining R₅-R₈ are hydrogen. In some embodiments, two of R₅, R₆, R₇, and R₈ are hydrogen, —Cl or —F and the remaining R₅-R₈ are hydrogen. In some embodiments, two of R₅, R₆, R₇, and R₈ are hydrogen or —F and the remaining R₅-R₈ are hydrogen.

In some embodiments, one of R₅, R₆, and R₇, is hydrogen, (C₁-C₃)-alkyl, —CN or halogen and the remaining R₅-R₇ are hydrogen and R₈ is hydrogen. In some embodiments, one of R₅, R₆, and R₇ is hydrogen, —Cl or —F, R₈ is hydrogen and the remaining R₅-R₇ are hydrogen. In some embodiments, one of R₅, R₆, and R₇ is hydrogen or —F, R₈ is hydrogen, and the remaining R₅-R₇ are hydrogen.

In some embodiments, two of R₅, R₆, and R₇, is hydrogen, (C₁-C₃)-alkyl, or halogen and the remaining R₅-R₇ is hydrogen and R₈ is hydrogen. In some embodiments, two of R₅, R₆, and R₇ are hydrogen, —Cl or —F, R₈ is hydrogen, and the remaining R₅-R₇ is hydrogen. In some embodiments, two of R₅, R₆, and R₇ are hydrogen or —F, R₈ is hydrogen, and the remaining R₅-R₇ is hydrogen.

In some embodiments, R₉ is hydrogen, methyl or ethyl.

In some embodiments, R₁₀, R₁₁, and R₁₂ are each independently hydrogen, (C₁-C₃)-alkyl, —CN or halogen. In some embodiments, R₁₀, R₁₁, and R₁₂ are each independently hydrogen or (C₁-C₃)-alkyl. In some embodiments, R₁₀, R₁₁, and R₁₂ are each hydrogen.

In some embodiments, R₉, R₁₀, R₁₁, and R₁₂ are each hydrogen.

In some embodiments, when n is 2, each R₁₀ is independently hydrogen, (C₁-C₃)-alkyl, —CN or halogen. In some embodiments, when n is 2, each R₁₀ is independently hydrogen or (C₁-C₃)-alkyl.

In some embodiments, R₁₄ and R₁₅ are each independently hydrogen or (C₁-C₆) alkyl. In some embodiments, R₁₄ and R₁₅ are each independently hydrogen or (C₁-C₃)alkyl. In some embodiments, R₁₄ is hydrogen or (C₁-C₃)alkyl and R₁₅ is hydrogen.

In some embodiments, R₁ is hydrogen or (C₁-C₆)-alkyl, R₂ is hydrogen, halogen, —CN, —CF₃, (C₁-C₆)-alkyl, or OR₁₄, and R₁₄ is hydrogen or (C₁-C₃)-alkyl.

In some embodiments, n is 1 or 2.

In some embodiments, X is —CR₁₃═CH— and R₁₃ is hydrogen or (C₁-C₃)-alkyl, R₁ is hydrogen or (C₁-C₆)-alkyl, R₂ is hydrogen, halogen, (C₁-C₆)-alkyl, —CN, —CF₃, or —OR₁; or R₁ and R₂, when taken together with the atoms to which they are attached and the carbon interposed between them, form a 5- to 7-membered unsaturated ring, optionally containing O; R₃ is hydrogen (C₁-C₆)-alkyl, or —NR₁₄, R₄ is hydrogen, halogen, (C₁-C₆)-alkyl, —CN, —CF₃, or —OR₁₄; R₅, R₆, R₇, and R₈ are each independently hydrogen, halogen, (C₁-C₃)-alkyl, —OR₁₄, —CN, or —C(O)NR₁₄; R₉ is hydrogen or (C₁-C₃)-alkyl; R₁₀, R₁₁, and R₁₂ are each independently hydrogen, halogen, or (C₁-C₆)-alkyl; R₁₄ is hydrogen or (C₁-C₆)-alkyl; and n is 1 or 2.

In some embodiments, X is —CR₁₃═CH— in which R₁₃ is hydrogen, R₁ is hydrogen or (C₁-C₃)-alkyl; R₂ is hydrogen, (C₁-C₃)-alkyl, or halogen; or R₁ and R₂, when taken together with the atoms to which they are attached and the carbon interposed between them, form a 5- to 7-membered unsaturated ring, optionally containing O; R₃ is hydrogen or —CH₃; R₅, R₆, R₇ and R₈ are each independently hydrogen or halogen; R₉, R₁₀, R₁₁, and R₁₂ are each independently hydrogen or (C₁-C₃)-alkyl; and n is 1 or 2.

In another embodiment, compounds of Formula Ia are provided:

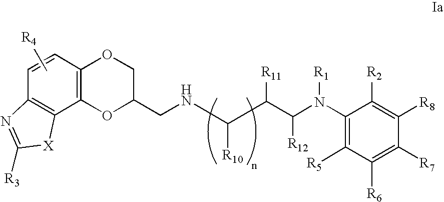

or an enantiomer, diastereomer, tautomer, or pharmaceutically acceptable salt or solvate thereof, wherein the R-groups, X and n are as defined herein.

In some embodiments, the compound of Formula I is a compound of Formula Ib:

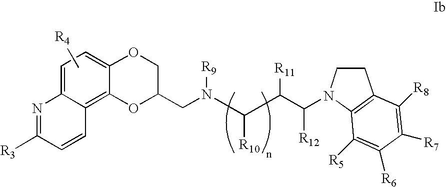

or an enantiomer, diastereomer, tautomer, or pharmaceutically acceptable salt or solvate thereof, wherein the R-groups and n are as defined herein.

In some embodiments, the compound of Formula I is a compound of Formula Ic:

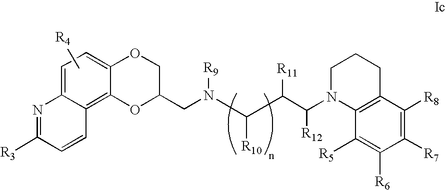

or an enantiomer, diastereomer, tautomer, or pharmaceutically acceptable salt or solvate thereof, wherein the R-groups and n are as defined herein.

In some embodiments, the compound of Formula I is a compound of Formula Id:

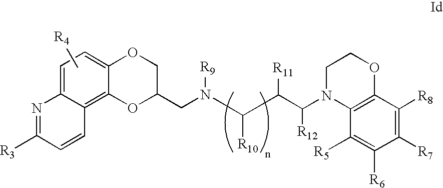

or an enantiomer, diastereomer, tautomer, or pharmaceutically acceptable salt or solvate thereof, wherein the R-groups and n are as defined herein.

In another embodiment, compounds of Formula Ie are provided:

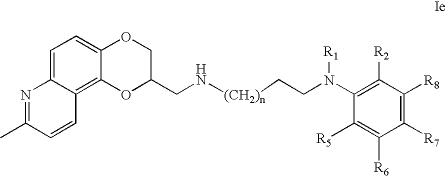

or an enantiomer, diastereomer, tautomer, or pharmaceutically acceptable salt or solvate thereof, wherein

R₁ is hydrogen or (C₁-C₃)-alkyl;

R₂ is hydrogen, (C₁-C₃)-alkyl, or halogen; or

R₁ and R₂, taken together with the atoms to which they are attached and the carbon interposed between them, form a 5- to 7-membered unsaturated ring, optionally containing O;

R₅, R₆, R₇, and R₈ are each independently hydrogen, —F or —Cl; and

n is 0, 1 or 2.

In some embodiments, two of R₅, R₆, R₇, and R₈ of the compound of Formula Ie are each independently hydrogen, —F or —Cl and the remaining R₅, R₆, R₇, or R₈ are hydrogen.

Specific examples of compounds of Formula I include, without limitation:
3-(3,4-dihydroquinolin-1(2H)-yl)-N-{[8-methyl-2,3-dihydro[1,4]dioxino[2,3-f]quinolin-2-yl]methyl}propan-1-amine
3-(5-fluoro-2,3-dihydro-1H-indol-1-yl)-N-{[8-methyl-2,3-dihydro[1,4]dioxino[2,3-f]quinolin-2-yl]methyl}propan-1-amine
3-(6-fluoro-2,3-dihydro-1H-indol-1-yl)-N-{[8-methyl-2,3-dihydro[1,4]dioxino[2,3-f]quinolin-2-yl]methyl}propan-1-amine
3-(7-fluoro-3,4-dihydroquinolin-1(2H)-yl)-N-{[8-methyl-2,3-dihydro[1,4]dioxino[2,3-f]quinolin-2-yl]methyl}propan-1-amine
3-(2,3-dihydro-1H-indol-1-yl)-N-{[8-methyl-2,3-dihydro[1,4]dioxino[2,3-f]quinolin-2-yl]methyl}propan-1-amine
3-(2,3-dihydro-4H-1,4-benzoxazin-4-yl)-N-{[8-methyl-2,3-dihydro[1,4]dioxino[2,3-f]quinolin-2-yl]methyl}propan-1-amine
3-(6,7-difluoro-3,4-dihydroquinolin-1(2H)-yl)-N-{[8-methyl-2,3-dihydro[1,4]dioxino[2,3-f]quinolin-2-yl]methyl}propan-1-amine
3-(6-fluoro-3,4-dihydroquinolin-1(2H)-yl)-N-{[8-methyl-2,3-dihydro[1,4]dioxino[2,3-f]quinolin-2-yl]methyl}propan-1-amine
3-(6,8-difluoro-3,4-dihydroquinolin-1(2H)-yl)-N-{[8-methyl-2,3-dihydro[1,4]dioxino[2,3-f]quinolin-2-yl]methyl}propan-1-amine
3-(6-fluoro-2,3-dihydro-4H-1,4-benzoxazin-4-yl)-N-{[8-methyl-2,3-dihydro[1,4]dioxino[2,3-f]quinolin-2-yl]methyl}propan-1-amine
N-{[8-methyl-2,3-dihydro[1,4]dioxino[2,3-f]quinolin-2-yl]methyl}-3-(2,3,4,5-tetrahydro-1H-1-benzazepin-1-yl)propan-1-amine
N-(2-chlorophenyl)-N-methyl-N′-{[8-methyl-2,3-dihydro[1,4]dioxino[2,3-f]quinolin-2-yl]methyl}propane-1,3-diamine
4-(6-fluoro-2,3-dihydro-1H-indol-1-yl)-N-{[8-methyl-2,3-dihydro[1,4]dioxino[2,3-f]quinolin-2-yl]methyl}butan-1-amine
4-(3,4-dihydroquinolin-1(2H)-yl)-N-{[8-methyl-2,3-dihydro[1,4]dioxino[2,3-f]quinolin-2-yl]methyl}butan-1-amine
4-(5-fluoro-2,3-dihydro-1H-indol-1-yl)-N-{[8-methyl-2,3-dihydro[1,4]dioxino[2,3-f]quinolin-2-yl]methyl}butan-1-amine or

an enantiomer, diastereomer, tautomer, or pharmaceutically acceptable salt or solvate thereof.

The compounds and pharmaceutically acceptable salts of compounds described herein can contain an asymmetric carbon atom and some of the compounds or pharmaceutically acceptable salts of compounds can contain one or more asymmetric centers, and can thus give rise to optical isomers and diastereomers. The embodiments described herein relate to both the R and S stereoisomers of the compounds described herein as well as to mixtures of the R and S stereoisomers. Throughout this application, the name of compounds, where the absolute configuration of the compounds is not indicated, is intended to embrace the individual R and S enantiomers as well as mixtures of the two. In some embodiments, the compounds described herein include the S enantiomer. Certain of the compounds described herein contain two stereogenic centers and thus may exist as diastereomers. In some embodiments, the compounds include one or both diastereomers, as well as mixtures of diastereomers. For example, specific examples of compounds of Formula I include, without limitation:
3-(3,4-dihydroquinolin-1(2H)-yl)-N-{[(2S)-8-methyl-2,3-dihydro[1,4]dioxino[2,3-f]quinolin-2-yl]methyl}propan-1-amine
3-(5-fluoro-2,3-dihydro-1H-indol-1-yl)-N-{[(2S)-8-methyl-2,3-dihydro[1,4]dioxino[2,3-f]quinolin-2-yl]methyl}propan-1-amine
3-(6-fluoro-2,3-dihydro-1H-indol-1-yl)-N-{[(2S)-8-methyl-2,3-dihydro[1,4]dioxino[2,3-f]quinolin-2-yl]methyl}propan-1-amine
3-(7-fluoro-3,4-dihydroquinolin-1(2H)-yl)-N-{[(2S)-8-methyl-2,3-dihydro[1,4]dioxino[2,3-f]quinolin-2-yl]methyl}propan-1-amine
3-(2,3-dihydro-1H-indol-1-yl)-N-{[(2S)-8-methyl-2,3-dihydro[1,4]dioxino[2,3-f]quinolin-2-yl]methyl}propan-1-amine
3-(2,3-dihydro-4H-1,4-benzoxazin-4-yl)-N-{[(2S)-8-methyl-2,3-dihydro[1,4]dioxino[2,3-f]quinolin-2-yl]methyl}propan-1-amine
3-(6,7-difluoro-3,4-dihydroquinolin-1(2H)-yl)-N-{[(2S)-8-methyl-2,3-dihydro[1,4]dioxino[2,3-f]quinolin-2-yl]methyl}propan-1-amine
3-(6-fluoro-3,4-dihydroquinolin-1(2H)-yl)-N-{[(2S)-8-methyl-2,3-dihydro[1,4]dioxino[2,3-f]quinolin-2-yl]methyl}propan-1-amine
3-(6,8-difluoro-3,4-dihydroquinolin-1(2H)-yl)-N-{[(2S)-8-methyl-2,3-dihydro[1,4]dioxino[2,3-f]quinolin-2-yl]methyl}propan-1-amine
3-(6-fluoro-2,3-dihydro-4H-1,4-benzoxazin-4-yl)-N-{[(2S)-8-methyl-2,3-dihydro[1,4]dioxino[2,3-f]quinolin-2-yl]methyl}propan-1-amine
N-{[(2S)-8-methyl-2,3-dihydro[1,4]dioxino[2,3-f]quinolin-2-yl]methyl}-3-(2,3,4,5-tetrahydro-1H-1-benzazepin-1-yl)propan-1-amine
N-(2-chlorophenyl)-N-methyl-N′-{[(2S)-8-methyl-2,3-dihydro[1,4]dioxino[2,3-f]quinolin-2-yl]methyl}propane-1,3-diamine
4-(6-fluoro-2,3-dihydro-1H-indol-1-yl)-N-{[(2S)-8-methyl-2,3-dihydro[1,4]dioxino[2,3-f]quinolin-2-yl]methyl}butan-1-amine
4-(3,4-dihydroquinolin-1(2H)-yl)-N-{[(2S)-8-methyl-2,3-dihydro[1,4]dioxino[2,3-f]quinolin-2-yl]methyl}butan-1-amine
4-(5-fluoro-2,3-dihydro-1H-indol-1-yl)-N-{[(2S)-8-methyl-2,3-dihydro[1,4]dioxino[2,3-f]quinolin-2-yl]methyl}butan-1-amine or

an enantiomer, diastereomer, tautomer, or pharmaceutically acceptable salt or solvate thereof.

In some embodiments, the stereoisomer is substantially free of the corresponding enantiomer. Thus, an enantiomer substantially free of the corresponding enantiomer refers to a compound which is isolated or separated via separation techniques or prepared free of the corresponding enantiomer. In some embodiments, stereoisomers are isolated from racemic mixtures by any method known to those skilled in the art, including high performance liquid chromatography (HPLC) and the formation and crystallization of chiral salts or prepared by methods described herein. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972).

In some embodiments, the compounds and pharmaceutically acceptable salts of compounds of the present invention exist as tautomers. Such tautomers can be transient or isolatable as a stable product. These tautomers are within the scope of the compounds described herein. For example, when X is —NR₁₃— and R₁₃ is hydrogen, two tautomeric forms of the compounds of Formula I; both are within the scope of the invention, as well as mixtures of the two.

Prodrugs of the compounds or pharmaceutically acceptable salts of compounds are also within the scope of the present invention.

The compounds and pharmaceutically acceptable salts of compounds described herein can be prepared using a variety of methods starting from commercially available compounds, known compounds, or compounds prepared by known methods. General synthetic routes to many of the compounds described herein are included in the following Schemes. The methods for making some intermediates are described in U.S. Pat. Nos. 6,800,637, 6,458,802, 6,525,075, 6,573,283, 6,599,915, 6,613,913, 6,617,327, 6,617,334, 6,656,947, 6,780,860, 6,803,368, 6,815,448 and 6,911,445 each of which is incorporated in its entirety by reference. It is understood by those skilled in the art that protection and deprotection steps not shown in the Schemes may be required for these syntheses, and that the order of steps may be changed to accommodate functionality in the target molecule. The need for protection and deprotection, and the selection of appropriate protecting groups can be readily determined by one skilled in the art. The chemistry of protecting groups can be found, for example, in Greene et al., Protective Groups in Organic Synthesis, 2d. ed., Wiley & Sons, 1991, which is incorporated herein by reference in its entirety.

In some embodiments, there is provided a method of synthesizing a compound of Formula I:

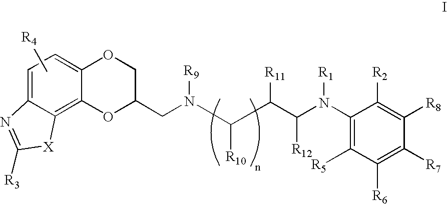

or an enantiomer, diastereomer, tautomer, pharmaceutically acceptable salt, or solvate thereof, wherein:

X is —O—, —N═CH—, —CR₁₃═CH—, —CR₁₃═N—, —CH═N—, —CH═CR₁₃—, —N═CR₁₃— or —NR₁₃—, in which R₁₃ is hydrogen or (C₁-C₆)-alkyl;

R₁ is hydrogen, (C₁-C₆)-alkyl, (C₁-C₆)-haloalkyl, (C₂-C₆)-alkenyl, or (C₂-C₆)-alkynyl;

R₂ is hydrogen, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₁₄, —OSO₂R₁₄, —SR₁₄, —SO₂R₁₄, —SO₂NR₁₄R₁₅, —NR₁₄R₁₅, —C(O), —COR₁₄, —CO₂R₁₄, —NR₁₄CO₂R₁₅, —NR₁₄COR₁₅, —NR₁₄CONR₁₅, —NR₁₄SO₂R₁₅, or —CONR₁₄R₁₅; or

R₁ and R₂, when taken together with the atoms to which they are attached and the carbon interposed between them, form a 5- to 7-membered saturated ring, optionally containing an additional heteroatom selected from O, NR₁₄ or SOₘ;

R₃ is hydrogen, (C₁-C₆)-alkyl, halogen, or NR₁₄R₁₅;

R₄ is hydrogen, (C₁-C₆)-alkyl, halogen, —CF₃, —CN, —OR₁₄, —SO₂R₁₄, —NR₁₄SO₂R₁₅, —NR₁₄R₁₅, —COR₁₄, —CO₂R₁₄, —NR₁₄COR₁₅, or —CONR₁₄R₁₅;

R₉ is hydrogen or (C₁-C₃)-alkyl;

R₅, R₆, R₇, R₈, R₁₀, R₁₁ and R₁₂ are each independently hydrogen, halogen, (C₁-C₃)-alkyl, (C₁-C₃)-haloalkyl, (C₂-C₃)-alkenyl, or (C₂-C₃)-alkynyl, —CN, —CF₃, —NO₂, —CN, —C(O)NR₁₄ or —OR₁₄;

R₁₄ and R₁₅ are each independently is hydrogen, (C₁-C₆)-alkyl, (C₁-C₆)-haloalkyl, (C₂-C₆)-alkenyl, or (C₂-C₆)-alkynyl; and

n and m are each independently 0, 1, or 2;

wherein when X is —O—, R₁ and R₂ are taken together with the atoms to which they are attached and the carbon interposed between them to form a 5- to 7-membered saturated ring, optionally containing an additional heteroatom selected from O, NR₁₄ or SOₘ; the method comprising:

reacting a compound of Formula 1, or an enantiomer, diastereomer, tautomer, pharmaceutically acceptable salt or solvate thereof,

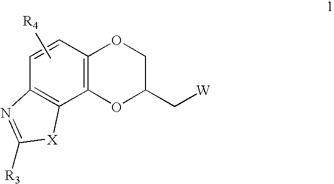

wherein X, R₃ and R₄ are as defined hereinabove, and W is a leaving group;

with an amino alcohol of Formula 2, or an enantiomer, diastereomer, tautomer, or pharmaceutically acceptable salt or solvate thereof,

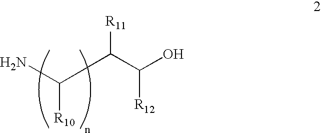

wherein R₁₀, R₁₁, R₁₂, and n are as defined hereinabove,

under conditions effective to produce a compound of Formula 3, or an enantiomer, diastereomer, tautomer, pharmaceutically acceptable salt or solvate thereof,

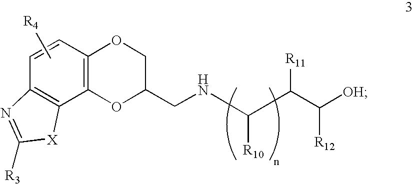

wherein X, R₃, R₄, R₁₀, R₁₁, R₁₂, and n are as defined hereinabove,

protecting the amino nitrogen of the compound of Formula 3, or an enantiomer, diastereomer, tautomer, or pharmaceutically acceptable salt or solvate thereof, under conditions effective to produce a compound of Formula 4, or an enantiomer, diastereomer, tautomer, or pharmaceutically acceptable salt or solvate thereof,

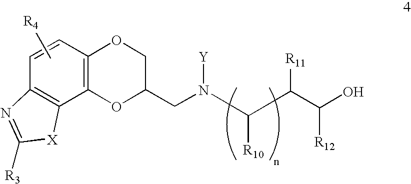

wherein X, R₃, R₄, R₁₀, R₁₁, R₁₂, and n are as defined hereinabove, and Y is an amino protecting group;

oxidizing the alcohol of the compound of formula 4, or an enantiomer, diastereomer, tautomer, or pharmaceutically acceptable salt or solvate thereof, under conditions effective to produce a compound of Formula 5, or an enantiomer, diastereomer, tautomer, or pharmaceutically acceptable salt or solvate thereof,

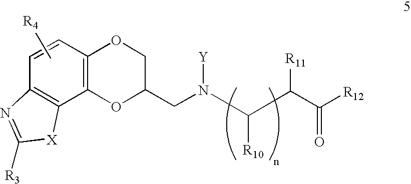

wherein X, R₃, R₄, R₁₀, R₁₁, R₁₂, Y, and n are as defined hereinabove,

reacting the compound of Formula 5, or an enantiomer, diastereomer, tautomer, or pharmaceutically acceptable salt or solvate thereof, with a compound of Formula 6, or an enantiomer, diastereomer, tautomer, pharmaceutically acceptable salt or solvate thereof,

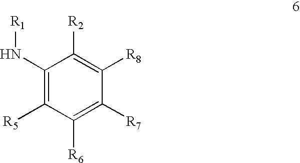

wherein R₁, R₂, R₅, R₆, R₇, and R₈ are as defined hereinabove,

under conditions effective to bring about reductive amination at the carbonyl of the compound of Formula 5, or an enantiomer, diastereomer, tautomer, or pharmaceutically acceptable salt or solvate thereof, thereby providing a compound of Formula 7, or an enantiomer, diastereomer, tautomer, pharmaceutically acceptable salt or solvate thereof,

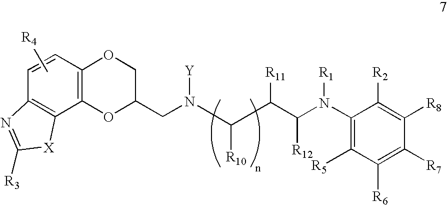

wherein X, R₁, R₂, R₃, R₄, R₅, R₆, R₇, R₈, R₁₀, R₁₁, R₁₂, Y, and n are as defined hereinabove,

reacting the compound of Formula 7, or an enantiomer, diastereomer, tautomer, pharmaceutically acceptable salt or solvate thereof, under conditions effective to remove the protecting group Y, thereby providing a compound having Formula Ia, or an enantiomer, diastereomer, tautomer, or pharmaceutically acceptable salt or solvate thereof,

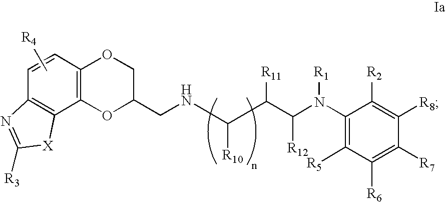

wherein X, R₁, R₂, R₃, R₄, R₅, R₆, R₇, R₈, R₁₀, R₁₁, R₁₂, and n are as defined hereinabove,

reacting the compound of Formula Ia, or an enantiomer, diastereomer, tautomer, pharmaceutically acceptable salt or solvate thereof, with a compound of Formula 8:

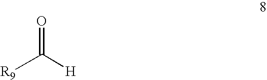

wherein R₉ is hydrogen or (C₁-C₃)-alkyl,

under conditions effective to bring about reductive amination at the nitrogen of the compound of Formula Ia, or an enantiomer, diastereomer, tautomer, pharmaceutically acceptable salt or solvate thereof, thereby providing a compound of Formula I, or an enantiomer, diastereomer, tautomer, pharmaceutically acceptable salt or solvate thereof.

In some embodiments, W is brosylate, tosylate, or mesylate. In some other embodiments, Y is t-BOC, or CBZ. In some embodiments, X is —CR₁₃═CH— and R₁₃ is hydrogen or (C₁-C₃)-alkyl. In some other embodiments, R₃ is hydrogen, —NH₂, or (C₁-C₆)-alkyl. In some embodiments, R₃ is hydrogen, —NH₂, or (C₁-C₆)-alkyl.

In some embodiments, X is —CR₁₃═CH— and R₃ is (C₁-C₆)-alkyl. In some other embodiments, R₄ is hydrogen, halogen, —CN, —CF₃, (C₁-C₆)-alkyl, or OR₁₄; and R₁₄ is hydrogen or (C₁-C₆)-alkyl. In some embodiments, R₁ is hydrogen or (C₁-C₆)-alkyl. In some other embodiments, wherein R₂ is hydrogen, halogen or (C₁-C₆)-alkyl. In some embodiments, R₁ and R₂ are taken together with the atoms to which they are attached and the carbon interposed between them to form a 5- to 7-membered saturated ring, optionally containing an additional heteroatom selected from O, NR₁₄ or SOₘ.

In some embodiments, R₅, R₆, R₇, and R₈ are each independently hydrogen, halogen, (C₁-C₃)-alkyl, —OR₂₅, —CN, or —C(O)NR₂₅ and R₂₅ is hydrogen or (C₁-C₆)-alkyl. In some embodiments, R₉ is methyl or ethyl. In some embodiments, R₁₀, R₁₁ and R₁₂ are each independently hydrogen, (C₁-C₃)-alkyl, or halogen. In some other embodiments, n is 1 or 2.

In some embodiments, X is —CR₁₃═CH— and in which R₁₃ is hydrogen or (C₁-C₃)-alkyl; R₁ is hydrogen or (C₁-C₆)-alkyl; R₂ is hydrogen, halogen, (C₁-C₆)-alkyl, —CN, —CF₃, or —OR₁₄, or R₁ and R₂, when taken together with the atoms to which they are attached and the carbon interposed between them, form a 5- to 7-membered unsaturated ring, optionally containing O; R₃ is hydrogen, (C₁-C₆)-alkyl, or —NR₁₄R₁₅; R₄ is hydrogen, halogen, (C₁-C₆)-alkyl, —CN, —CF₃, or —OR₁₄; R₅, R₆, R₇, and R₈ are each independently hydrogen, halogen, (C₁-C₃)-alkyl, —OR₁₄, —CN, or —C(O)NR₁₄; R₉ is hydrogen or (C₁-C₃)-alkyl; R₁₀, R₁₁, and R₁₂ are each independently hydrogen, halogen, or (C₁-C₃)-alkyl; R₁₄ is hydrogen or (C₁-C₆)-alkyl; and n is 1 or 2.

In some embodiments, X is —CR₁₃═CH— and in which R₁₃ is hydrogen; R₁ is hydrogen or (C₁-C₃)-alkyl; R₂ is hydrogen, (C₁-C₃)-alkyl, or halogen; or R₁ and R₂, when taken together with the atoms to which they are attached and the carbon interposed between them, form a 5- to 7-membered unsaturated ring, optionally containing O; R₃ is hydrogen or —CH₃; R₅, R₆, R₇ and R₈ are each independently hydrogen or halogen; R₉, R₁₀, R₁₁, and R₁₂ are each independently hydrogen or (C₁-C₃)-alkyl; and n is 1 or 2.

In some embodiments, there is provided a method of synthesizing a compound of Formula Ie, or an enantiomer, diastereomer, tautomer, or pharmaceutically acceptable salt or solvate thereof,

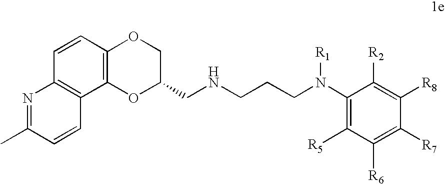

wherein:

R₁ is hydrogen, (C₁-C₆)-alkyl, (C₁-C₆)-haloalkyl, (C₂-C₆)-alkenyl, or (C₂-C₆)-alkynyl;

R₂ is hydrogen, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₁₄, —OSO₂R₁₄, —SR₁₄, —SO₂R₁₄, —SO₂NR₁₄R₁₅, —NR₁₄R₁₅, —C(O), —COR₁₄, —CO₂R₁₄, —NR₁₄CO₂R₁₅, —NR₁₄COR₁₅, —NR₁₄CONR₁₅, —NR₁₄SO₂R₁₅, or —CONR₁₄R₁₅; or

R₁ and R₂, when taken together with the atoms to which they are attached and the carbon interposed between them, form a 5- to 7-membered saturated ring, optionally containing an additional heteroatom selected from O, NR₁₄ or SOₘ;

R₅, R₆, R₇, and R₈ are each independently hydrogen, halogen, (C₁-C₃)-alkyl, (C₁-C₃)-haloalkyl, (C₂-C₃)-alkenyl, or (C₂-C₃)-alkynyl, —CN, —CF₃, —NO₂, —CN, —C(O)NR₁₄ or —OR₁₄;

R₁₄ and R₁₅ are each independently is hydrogen, (C₁-C₆)-alkyl, (C₁-C₆)-haloalkyl, (C₂-C₆)-alkenyl, or (C₂-C₆)-alkynyl; and

n and m are each independently 0, 1, or 2;

the method comprising:

reacting a compound of Formula Ie, or an enantiomer, diastereomer, tautomer, or pharmaceutically acceptable salt or solvate thereof,

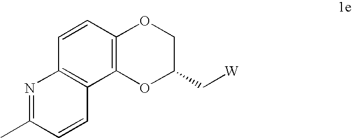

wherein W is a leaving group;

with an amino alcohol of Formula 2e, or an enantiomer, diastereomer, tautomer, or pharmaceutically acceptable salt or solvate thereof,

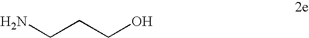

under conditions effective to produce a compound of Formula 3e, or an enantiomer, diastereomer, tautomer, or pharmaceutically acceptable salt or solvate thereof,

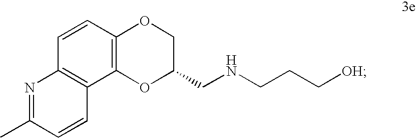

protecting the amino nitrogen of the compound of Formula 3e, or an enantiomer, diastereomer, tautomer, or pharmaceutically acceptable salt or solvate thereof, under conditions effective to produce a compound of Formula 4e, or an enantiomer, diastereomer, tautomer, or pharmaceutically acceptable salt or solvate thereof,

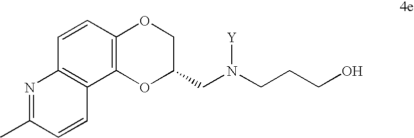

wherein Y is an amino protecting group;

oxidizing the alcohol of the compound of formula 4e, or an enantiomer, diastereomer, tautomer, or pharmaceutically acceptable salt or solvate thereof, under conditions effective to produce a compound of Formula 5e, or an enantiomer, diastereomer, tautomer, or pharmaceutically acceptable salt or solvate thereof,

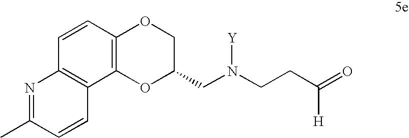

wherein Y is as defined hereinabove,

reacting the compound of Formula 5e, or an enantiomer, diastereomer, tautomer, or pharmaceutically acceptable salt or solvate thereof, with a compound of Formula 6e, or an enantiomer, diastereomer, tautomer, or pharmaceutically acceptable salt or solvate thereof,

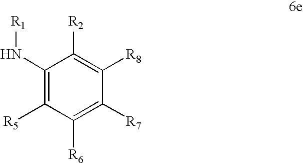

wherein R₁, R₂, R₅, R₆, R₇, and R₈ are as defined hereinabove,

under conditions effective to bring about reductive amination at the carbonyl of the compound of Formula 5, or an enantiomer, diastereomer, tautomer, or pharmaceutically acceptable salt or solvate thereof, thereby providing a compound of Formula 7e, or an enantiomer, diastereomer, tautomer, or pharmaceutically acceptable salt or solvate thereof,

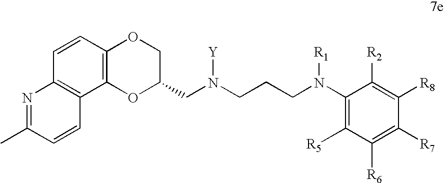

wherein R₁, R₂, R₅, R₆, R₇, R₈ and Y are as defined hereinabove,

reacting the compound of Formula 7e, or an enantiomer, diastereomer, tautomer, or pharmaceutically acceptable salt or solvate thereof, under conditions effective to remove the protecting group Y, thereby providing a compound having Formula Ie, or an enantiomer, diastereomer, tautomer, or pharmaceutically acceptable salt or solvate thereof.

In some embodiments, W is brosylate, tosylate, or mesylate. In some other embodiments, Y is t-BOC or CBZ.

In some embodiments, R₁ is hydrogen or (C₁-C₆)-alkyl; R₂ is hydrogen, halogen, (C₁-C₆)-alkyl, —CN, —CF₃, or —OR₁₄, or R₁ and R₂, when taken together with the atoms to which they are attached and the carbon interposed between them, form a 5- to 7-membered unsaturated ring, optionally containing O; R₅, R₆, R₇, and R₈ are each independently hydrogen, halogen, (C₁-C₃)-alkyl, —OR₁₄, —CN, or —C(O)NR₁₄; and R₁₄ is hydrogen or (C₁-C₆)-alkyl.

In some embodiments, R₁ is hydrogen; R₂ is hydrogen, (C₁-C₃)-alkyl, or halogen; or R₁ and R₂, when taken together with the atoms to which they are attached and the carbon interposed between them, form a 5- to 7-membered unsaturated ring, optionally containing O; and R₅, R₆, R₇ and R₈ are each independently hydrogen or halogen.

Scheme 1 illustrates one process for making compounds of Formula I, where the R groups and other groups are as defined herein. As shown in Scheme 1, compound I containing a leaving group, W (e.g., brosylate, tosylate, mesylate, nosylate, halogen, alkoxy, sulphonyloxy, optionally substituted alkylsulphonyloxy, optionally substituted alkenylsulfonyloxy, optionally substituted arylsulfonyloxy or other leaving groups known to those skilled in the art (See, e.g., Jerry March, Advanced Organic Chemistry, 4th ed., John Wiley & Sons, New York, 1992)) is reacted with an amino alcohol 2 (e.g., amino propanol, amino ethanol, or amino methanol). In some embodiments, W is brosylate, tosylate, or mesylate. The compounds are reacted under conditions effective to produce the corresponding amino alcohol derivative 3. For example, in some embodiments, the reaction proceeds in a polar solvent (e.g., dimethylsulfoxide (DMSO) or DMF) with added heat (e.g., about 50-150° C. for several hours or about 150° C. in a microwave reactor for about 10-30 minutes). The amino alcohol derivative 3 is subsequently protected with a protecting group Y (e.g., a t-butyl moiety such as t-BOC, CBZ, benzyl, tetrahydropyran, or other secondary amine protecting groups known to those of skill in the art) to produce compound 4. In some embodiments, Y is Y is t-BOC, or CBZ. The alcohol 4 is then oxidized under conditions effective to produce the corresponding compound 5. Oxidizing agents known to those of skill in the art, such as Dess-Martin periodinane, Swem oxidation agents (where a primary or secondary alcohol is oxidized to an aldehyde or ketone using oxalyl chloride, dimethyl sulfoxide (DMSO) and an organic base, such as triethylamine, see, e.g., Omura, K.; Swem, D. Tetrahedron 1978, 34, 1651; Mancuso, A. J.; Huang, S.-L.; Swem, D. J. Org. Chem. 1978, 43, 2480-2482; Mancuso, A. J.; Brownfain, D. S.; Swem, D. J. Org. Chem. 1979, 44, 4148.), or PDC are used to accomplish the oxidation reaction. Compound 5 is reacted with substituted aniline 6 under conditions effective to bring about subsequent deprotection affording compound Ia where R₁, R₂, R₃, R₄, R₅, R₆, R₇, R₈, R₁₀, R₁₁, R₁₂, X, and n are as defined herein. Where further substitution of the unsubstituted nitrogen of compound Ia is desired, a second reductive amination with compound 8, wherein R₉ is hydrogen or (C₁₋₃)alkyl, followed by deprotection affords compound I where R₉ is as defined herein.

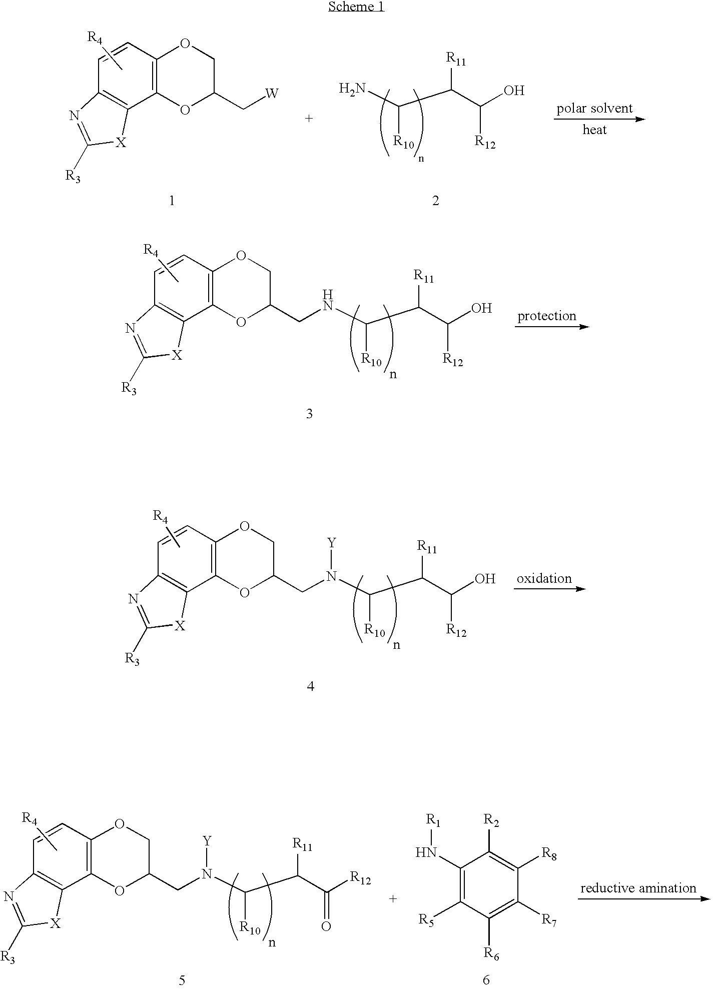

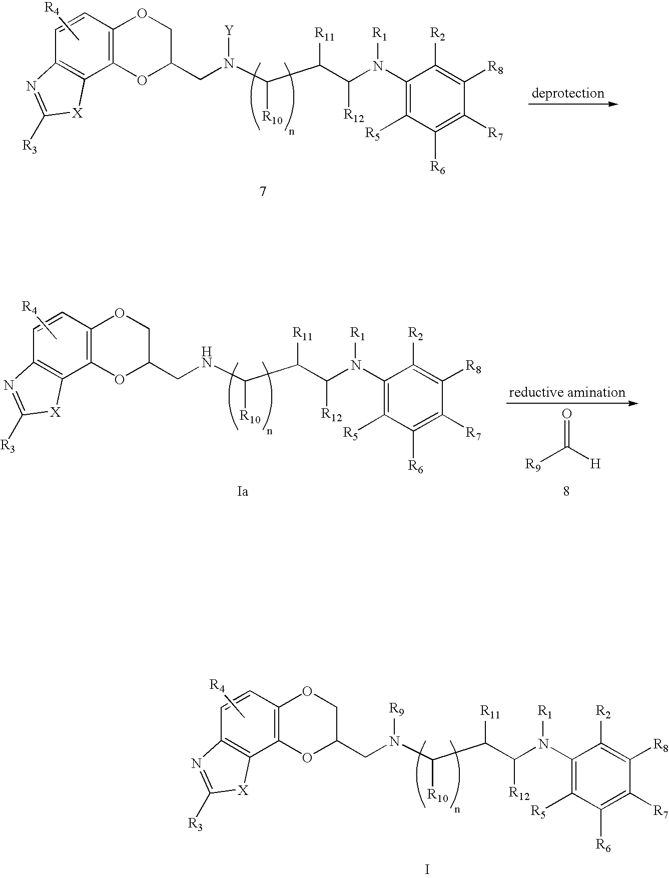

Scheme 2 illustrates an exemplary process for the preparation of compounds of formula Ie where n is 1. As shown in Scheme 2, a quinolinedioxane derivative Ie containing a leaving group W, such as a brosylate is allowed to react with an amino alcohol, such as amino propanol 2e, in a polar solvent, such as dimethylsulfoxide (DMSO) with heating (e.g., from 50-150° C. for several hours or at 150° C. in a microwave reactor for 10-30 minutes) to give the corresponding amino alcohol derivative 3e, which is subsequently protected with an amine protecting group Y (e.g., BOC) to give primary alcohol 4e. The primary alcohol 4e is then oxidized to the corresponding aldehyde 5e with an oxidizing agent such as Dess-Martin periodinane. Subsequent reductive amination with substituted aniline 6e affords compound 7e and deprotection affords the compound of Formula Ie where R₁, R₂, R₅-R₈, X, and Y are as defined in the generic structure.

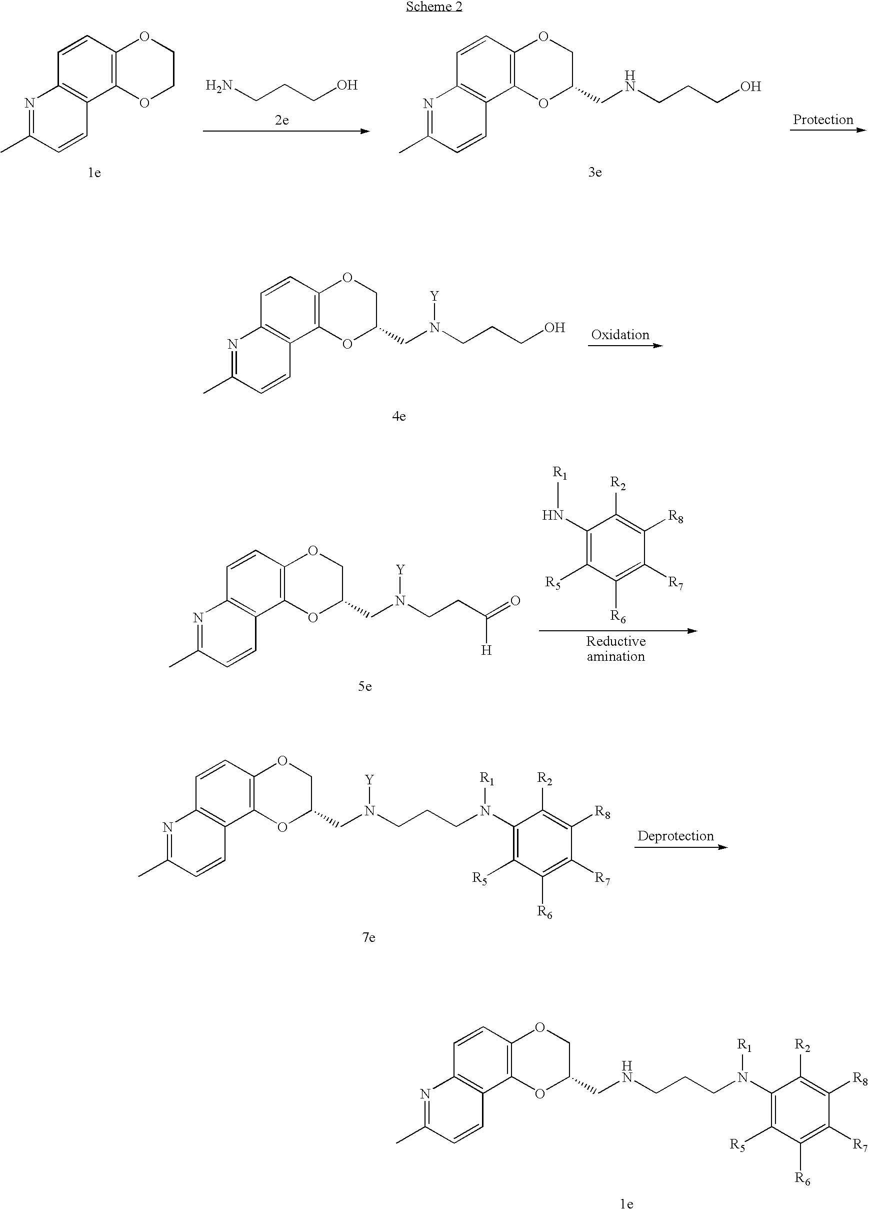

Synthetic intermediates useful for preparing the compounds described herein may be prepared according to methods known to those in the art or methods published elsewhere. In some embodiments, the substituted anilines described herein are either available from commercial sources or may be prepared according to the methods described in the examples described herein.

In some embodiments, the compounds described herein are further reacted to form a salt through an acid addition process. In one non-limiting example, one or more equivalents of an acid (e.g., hydrochloride, succinic, or adipic acids) are reacted with the free base of a compound described herein to form an acid addition salt. Exemplary salts include, without limitation, mono-, di-, tri- and tetra-acid salts.

Schemes 1 and 2 illustrate the synthetic methodology used to prepare particular compounds described herein. One of skill in the art will recognize that Schemes 1 and 2 can be adapted to produce the other compounds according to one or more embodiments described herein and that other methods may be used to produce the compounds described herein.

### Features of 5-HT1A Binding Compounds

The compounds disclosed herein have features that include the ability to bind 5-HT_{1A} receptors with high affinity, e.g., an affinity (Kᵢ) value of less than 100 nM. Methods of testing compounds for the ability to bind to a 5-HT_{1A} receptor are known in the art (for example, Watson et al., British J. Pharmacol. 130:1108-1114 (2000); Kim et al., Pharmacology 65:175-181 (2002); Atkinson et al., Bioorg. Med. Chem. Lett. 15:737-741 (2005)) Additional methods are illustrated in the Examples.

Compounds can also be tested for their ability to inhibit serotonin reuptake, and therefore act as an SSRI (for example, Atkinson et al., Bioorg. Med. Chem. Lett. 15:737-741 (2005) and Examples provided infra). Methods of testing for such function is known in the art and an example of a method for determining serotonin uptake is described in the Examples.

### Uses of Compounds

In general, the compounds disclosed herein (5-HT_{1A} binding compounds) are useful for modulating 5-HT activity, particularly by interacting with a 5-HT_{1A} receptor. Typically, the compounds disclosed herein act as partial 5-HT_{1A} agonists. In addition to their activity as 5-HT_{1A} agonists or antagonists, these compounds can also act as SSRIs. This capacity for dual functionality is a useful property of these compounds, e.g., for treating disorders such as cognitive or psychiatric disorders. For example, 5-HT_{1A} partial agonists have been shown to enhance cholinergic transmission and cognitive function in rodents (Millan, JPET Fast Forward (DOI: 10.1124/jpet.104.069625) (2004)), decrease anxiety in an animal model (Adamic et al., Eur. J. Pharmacol. 504:65-77 (2004)), improve behavioral and psychological symptoms of dementia (Sato, Int. J. Neuropsychopharmacol. 10:281-283 (2007); Epub 2006 Jul. 3).

Compounds that act as 5-HT_{1A} agonists and as SSRIs (dual function 5-HT_{1A} binding compounds) are also useful. For example, such compounds are useful for treating conditions that are treated with SSRIs alone. Treatment of a subject with a dual function 5-HT_{1A} binding compound can result in the subject having fewer undesirable side effects (e.g., less sexual dysfunction) than is experienced using an SSRI alone. Preclinical studies have demonstrated that the addition of a 5-HT_{1A} antagonist to an SSRI can reverse the sexual side effects produced by the SSRI alone, as measured by the number on noncontact penile observed in male rats. (Sukoff Rizzo et al. Psychopharmacology, published online Sep. 16, 2007). In addition, a dual function 5-HT_{1A} binding compound can have increased therapeutic activity, e.g., may decrease the amount of time to achieve a therapeutic effect) compared to treatment with an SSRI compound that does not have 5-HT_{1A} agonist activity.

Modulation of 5-HT activity is generally achieved by contacting a cell having a 5-HT receptor, generally a 5-HT_{1A} receptor, contacting the cell with a compound disclosed herein under conditions and for a time sufficient to elicit modulation of 5-HT activity. In some cases, the modulation is compared to a reference such as a corresponding cell that is not contacted with the compound. The cell can be a cultured cell, a cell in a culture tissue, or a cell in an animal (e.g., in a mammal, such as a human). As is known in the art, 5-HT activity can depend on the type of cell contacted with the compound, and methods of assaying such activity are known in the art. Types of activity that are modulated include, without limitation, effects associated with inhibition of serotonin reuptake (e.g., inhibition of a 5-HT transporter protein), modulation of cAMP levels in a cell (e.g., a decrease or an increase in cAMP levels), or modulation of the function of an ion channel in the cell. In some cases, modulation of 5-HT_{1A} activity is amelioration of a disorder associated with 5-HT_{1A}, as described herein.

### Pharmaceutical Formulations

In some embodiments, the compounds or pharmaceutically acceptable salts of the compounds described herein are a component of a composition that comprises one or more pharmaceutically acceptable vehicles, carriers, excipients, or diluents. Such pharmaceutical compositions can be prepared using a method comprising admixing the compound or pharmaceutically acceptable salt of the compound and a physiologically acceptable carrier, excipient, or diluent. Admixing can be accomplished using methods known for admixing a compound or a pharmaceutically acceptable salt of a compound and a physiologically acceptable carrier, excipient, or diluent. Examples of such carriers, excipients, and diluents are well known to those skilled in the art and are prepared in accordance with acceptable pharmaceutical procedures, such as, for example, those described in Remington's Pharmaceutical Sciences, 19th edition, ed. Alfonso R. Gennaro, Mack Publishing Company, Easton, Pa. (1995), which is incorporated herein by reference in its entirety. Pharmaceutically acceptable carriers, excipients, and diluents are those that are compatible with the other ingredients in the formulation and biologically acceptable.

Exemplary physiologically acceptable excipients include, without limitation, liquids such as water and oils, including those of petroleum, animal, vegetable, or synthetic origin, such as peanut oil, soybean oil, mineral oil, sesame oil and the like. Other non-limiting examples of physiologically acceptable excipients are saline, gum acacia, gelatin, starch paste, talc, keratin, colloidal silica, urea and the like. In addition, auxiliary, stabilizing, thickening, lubricating, and coloring agents can optionally be used. In some cases the physiologically acceptable excipients are sterile when administered to an animal. The physiologically acceptable excipient should be stable under the conditions of manufacture and storage and should be preserved against the contaminating action of microorganisms. Water is a particularly useful excipient when the compound or a pharmaceutically acceptable salt of the compound is administered intravenously. Saline solutions and aqueous dextrose and glycerol solutions can also be employed as liquid excipients, particularly for injectable solutions. Suitable physiologically acceptable excipients also include, without limitation, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, also optionally contain wetting or emulsifying agents, or pH buffering agents. Other examples of suitable physiologically acceptable excipients are described in Remington's Pharmaceutical Sciences 1447-1676 (Alfonso R. Gennaro, ed., 19th ed. 1995).

The present compositions can take the form of solutions, suspensions, emulsions, tablets, pills, pellets, capsules, capsules containing liquids, powders, sustained-release formulations, suppositories, aerosols, sprays, or any other form suitable for use. In some embodiments, the composition is in the form of a capsule.

The compounds or pharmaceutically acceptable salts of the compounds described herein may be administered orally or parenterally, neat or in combination with conventional pharmaceutical carriers as described above. The compounds or pharmaceutically acceptable salts of the compounds described herein can also be administered by any convenient route, for example, orally, by infusion or bolus injection, by absorption through epithelial or mucocutaneous linings (e.g., nasal, oral, rectal, vaginal, and intestinal mucosa) and can be administered together with another therapeutic agent. Other methods of administration include, but are not limited to, intradermal, intramuscular, intraperitoneal, intravenous, subcutaneous, intranasal, epidural, sublingual, intracerebral, intravaginal, transdermal, rectal, by inhalation, or topical, particularly to the ears, nose, eyes, or skin. Administration can be systemic or local. Various known delivery systems, including encapsulation in liposomes, microparticles, microcapsules, and capsules, can be used. For example, in some embodiments, the compounds or pharmaceutically acceptable salts of the compounds described herein can be delivered in a vesicle, in particular a liposome (see Langer, Science 249:1527-1533 (1990) and Treat et al., Liposomes in the Therapy of Infectious Disease and Cancer, pp. 317-327 and pp. 353-365 (1989)). In some instances, administration will result in release of the compound or a pharmaceutically acceptable salt of the compound into the bloodstream.

In some embodiments, the compound or pharmaceutically acceptable salt of the compound is administered orally.

In some cases, the compound or pharmaceutically acceptable salt of the compound is administered intravenously.

In some embodiments, the compounds or pharmaceutically acceptable salts of the compounds described herein are administered locally. This can be achieved, for example, by local infusion during surgery, topical application, e.g., in conjunction with a wound dressing after surgery, by injection, by means of a catheter, by means of a suppository or enema, or by means of an implant, said implant being of a porous, non-porous, or gelatinous material, including membranes, such as silastic membranes, or fibers.

In certain embodiments, it can be desirable to introduce the compounds or pharmaceutically acceptable salts of the compounds described herein into the central nervous system, circulatory system or gastrointestinal tract by any suitable route, including intraventricular, intrathecal injection, paraspinal injection, epidural injection, enema, and by injection adjacent to a peripheral nerve. Intraventricular injection can be facilitated by an intraventricular catheter, for example, attached to a reservoir, such as an Ommaya reservoir.

In some embodiments, the compound or a pharmaceutically acceptable salt of the compound is formulated in accordance with routine procedures as a composition adapted for oral administration to humans. For example, compositions for oral delivery can be in the form of tablets, lozenges, buccal forms, troches, aqueous or oily suspensions or solutions, granules, powders, emulsions, capsules, syrups, elixirs, oral liquids, suspensions or solutions. Orally administered compositions can contain one or more agents, for example, a sweetening agent such as fructose, aspartame or saccharin; flavoring agents such as peppermint, oil of wintergreen, or cherry; coloring agents; and preserving agents, to provide a pharmaceutically palatable preparation. Oral formulations may utilize standard delay or time release formulations to alter the absorption of the compound or pharmaceutically acceptable salt of the compound. An oral formulation may also be formulated such that the compound or pharmaceutically acceptable salt of the compound can be administered in a liquid such as water or fruit juice. Such compositions can contain appropriate solubilizers or emulsifiers as needed.

Applicable solid carriers can include one or more substances acting as flavoring agents, lubricants, solubilizers, suspending agents, fillers, glidants, compression aids, binders or tablet-disintegrating agents or encapsulating materials. They are formulated in conventional manner, for example, in a manner similar to that used for known antihypertensive agents, diuretics and β-blocking agents. In powders, the carrier is a finely divided solid, which is an admixture with the finely divided compound or pharmaceutically acceptable salt of the compound. In tablets, in some embodiments, the compound or pharmaceutically acceptable salt of the compound is mixed with a carrier having the necessary compression properties in suitable proportions and compacted in the shape and size desired. In some embodiments, a powder or tablet contains up to 99% of the compound or pharmaceutically acceptable salt of the compound.

Capsules can contain mixtures of the compounds or pharmaceutically acceptable salts of the compounds with inert fillers and/or diluents such as pharmaceutically acceptable starches (e.g. corn, potato or tapioca starch), sugars, artificial sweetening agents, powdered celluloses, (such as crystalline and microcrystalline celluloses), flours, gelatins, gums, and other pharmaceutically acceptable fillers and/or diluents known to those of skill in the art.

Tablet formulations can be made by conventional compression, wet granulation or dry granulation methods and utilize one or more of a pharmaceutically acceptable diluent, binding agent, lubricant, disintegrant, surface modifying agent (including surfactants), suspending or stabilizing agent, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, a low melting wax or ion exchange resin. In some embodiments, the surface modifying agent can include one or more of a nonionic and/or anionic surface modifying agent. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine.

The compound or a pharmaceutically acceptable salt of the compound can be administered by controlled-release or sustained-release means or by delivery devices that are known to those of ordinary skill in the art (see, e.g., Goodson, in Medical Applications of Controlled Release, vol. 2, pp. 115-138 (1984)). Such dosage forms can be used to provide controlled- or sustained-release of one or more compounds or pharmaceutically acceptable salts of the compounds using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the compounds or pharmaceutically acceptable salts of the compounds described herein. Thus, in some embodiments, the compounds or pharmaceutically acceptable salts of the compounds are provided as single unit dosage forms suitable for oral administration such as, but not limited to, tablets, capsules, gelcaps, and caplets that are adapted for controlled- or sustained-release. Other controlled- or sustained-release systems such as those discussed in Langer (Science 249:1527-1533 (1990)) or other methods known in the art can be used. In some cases, a pump is used (Langer, Science 249:1527-1533 (1990); Sefton, CRC Crit. Ref. Biomed. Eng. 14:201-240 (1987); Buchwald et al., Surgery 88:507-516 (1980); and Saudek et al., N. Engl. J. Med. 321:574-579 (1989)). In some cases, polymeric materials are used (for example, see, Medical Applications of Controlled Release (Langer and Wise eds., CRC Press, Boca Raton, Fla. (1984); Controlled Drug Bioavailability, Drug Product Design and Performance (Smolen and Ball eds., Wiley Interscience Publication, 1984); Langer and Peppas, J. Macromol. Sci. Rev. Macromol. Chem. 2:61-126 (1983); Levy et al., Science 228:190-192 (1985); During et al., Ann. Neurol. 25:351-356 (1989); and Howard et al., J. Neurosurg. 71:105-109 (1989)).

Similarly, when in a tablet or pill form, the compositions can be coated to delay disintegration and absorption in the gastrointestinal tract, thereby providing a sustained action over an extended period of time. Selectively permeable membranes surrounding an osmotically active driving compound or a pharmaceutically acceptable salt of the compound are also suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In some embodiments, the excipients are of pharmaceutical grade.

Liquid carriers may be used in preparing solutions, suspensions, emulsions, syrups and elixirs. A compound or pharmaceutically acceptable salt of a compound described herein can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include one or more of water (particularly containing additives as above, e.g., cellulose derivatives, such as a sodium carboxymethyl cellulose solution), an alcohol (including monohydric alcohols and polyhydric alcohols, e.g., glycols) and their derivatives, and oils (e.g., fractionated coconut oil or arachis oil). For parenteral administration the carrier can be an oily ester such as ethyl oleate or isopropyl myristate. Sterile liquid carriers are used in sterile liquid form compositions for parenteral administration. The liquid carrier for pressurized compositions can be halogenated hydrocarbon or other pharmaceutically acceptable propellant.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration may be in either liquid or solid form.

Pulmonary administration can also be employed, e.g., by use of an inhaler or nebulizer, and formulation with an aerosolizing agent, or via perfusion in a fluorocarbon or synthetic pulmonary surfactant. In some embodiments, the compounds or pharmaceutically acceptable salts of the compounds are administered directly to the airways in the form of an aerosol. For administration by intranasal or intrabronchial inhalation, the compounds or pharmaceutically acceptable salts of the compounds may be formulated into an aqueous solution, partially aqueous solution, or powder.

The compounds or pharmaceutically acceptable salts of the compounds described herein may be administered parenterally or intraperitoneally. Solutions or suspensions of these compounds or pharmaceutically acceptable salts may be prepared in water suitably mixed with a surfactant such as hydroxyl propylcellulose. Dispersions may also be prepared in glycerol, liquid polyethylene glycols and mixtures thereof in oils. Under typical conditions of storage and use, these preparations contain a preservative to inhibit the growth of microorganisms.

A pharmaceutical formulation of the invention suitable for injectable use can be, for example, a sterile aqueous solution or dispersion or sterile powder for the extemporaneous preparation of a sterile injectable solution or dispersion. In some embodiments, the formulation is sterile and is fluid to the extent that easy syringability exists. Such dosage forms are generally stable under the conditions of manufacture and storage and can be preserved against the contaminating action of microorganisms such as bacteria and fungi. In some embodiments, the carrier is a solvent or dispersion medium containing, for example, water, ethanol, polyol (e.g., glycerol, propylene glycol and liquid polyethylene glycol), suitable mixtures thereof, or vegetable oils.

In another embodiment, the compound or a pharmaceutically acceptable salt of the compound can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized powder or water-free concentrate in a hermetically sealed container such as an ampule or sachette indicating the quantity of the compound or pharmaceutically acceptable salt of the compound. Where the compound or a pharmaceutically acceptable salt of the compound is to be administered by infusion, it can be dispensed, for example, with an infusion bottle containing sterile pharmaceutical grade water or saline. Where the compound or a pharmaceutically acceptable salt of the compound is administered by injection, an ampule of sterile water for injection or saline can be provided so that the ingredients can be mixed prior to administration.

The compounds or pharmaceutically acceptable salts of the compounds described herein can be administered transdermally through the use of a transdermal patch. Transdermal administration includes administration across the surface of the body and the inner linings of bodily passages including epithelial and mucosal tissues. Such administration may be carried out using a compound described herein, or a pharmaceutically acceptable salt thereof, in a lotion, cream, foam, patch, suspension, solution, or suppository (rectal and vaginal).

Transdermal administration may be accomplished through the use of a transdermal patch containing the compound or pharmaceutically acceptable salt of the compound and a carrier that is inert to the compound, is non-toxic to the skin, and allows delivery of a compound described herein for systemic absorption into the bloodstream via the skin. The carrier may take any number of forms such as a cream or ointment, paste, gel or occlusive device. The creams and ointments may be viscous liquid or semisolid emulsions of either the oil-in-water or water-in-oil type. Pastes comprised of absorptive powders dispersed in petroleum or hydrophilic petroleum containing the compound or a pharmaceutically acceptable salt of the compound may also be suitable. A variety of occlusive devices may be used to release the compound or pharmaceutically acceptable salt of the compound into the blood stream, such as a semi-permeable membrane covering a reservoir containing the compound or pharmaceutically acceptable salt of the compound with or without a carrier, or a matrix containing the compound or pharmaceutically acceptable salt of the compound. Other occlusive devices are known in the literature.

The compounds or pharmaceutically acceptable salts of the compounds described herein may be administered rectally or vaginally in the form of a conventional suppository. Suppository formulations may be made from one or more binders and excipients known in the art, including cocoa butter and triglycerides, with or without the addition of waxes to alter the suppository's melting point, and glycerin. Water soluble suppository bases, such as polyethylene glycols of various molecular weights, may also be used.

It is understood that the dosage, regimen and mode of administration of the compounds described herein will vary according to the malady and the individual being treated and will be subject to the judgment of the medical practitioner involved. In some embodiments, administration of one or more of the compounds or pharmaceutically acceptable salts of the compounds described herein begins at a low dose and is increased until at least one desired effect is achieved.

When administered for the treatment or inhibition of a particular disease state or disorder, it is understood that the effective dosage may vary depending upon the particular compound or pharmaceutically acceptable salt of the compound utilized, the mode of administration, the condition, and severity thereof, of the condition being treated, as well as the various physical factors related to the individual being treated. In therapeutic application, the compounds and pharmaceutically acceptable salts of the compounds described herein are provided to a subject (i.e., a patient) already suffering from a disease in an amount sufficient to cure or at least partially ameliorate the symptoms of the disease and its complications. An amount adequate to accomplish this is defined as a “therapeutically effective amount”. The dosage to be used in the treatment of a specific case may be determined by the attending physician. The variables involved include the specific condition to be treated and the size, age and response pattern of the patient.

A controlled- or sustained-release composition can comprise a minimal amount of a compound or a pharmaceutically acceptable salt of the compound to treat or prevent a nervous system disorder, for example a disorder that is central nervous system disorder. In some cases, the nervous system disorder is a disorder that involves the peripheral nervous system. Typically, the disorder is a disorder mediated by a 5-HT_{1A} receptor and can involve, for example, a mutation in the 5-HT_{1A} or decreased expression of the receptor and/or if the disorder is dependent on serotonin transporter function. In some cases, such dosing results in a reduced amount of time for efficacy of the active compound compared to administration of the compound in compositions that are not so formulated, i.e., containing only the SSRI pharmacore. Advantages of controlled- or sustained-release compositions include extended activity of the drug, reduced dosage frequency, and increased compliance by the animal being treated. In addition, controlled or sustained release compositions can favorably affect the time of onset of action or other characteristics, such as blood levels of the compound or a pharmaceutically acceptable salt of the compound, and can thus reduce the occurrence of adverse side effects.

Controlled- or sustained-release compositions can initially release an amount of the compound or a pharmaceutically acceptable salt of the compound that promptly produces the desired therapeutic or prophylactic effect, and gradually and continually release other amounts of the compound or a pharmaceutically acceptable salt of the compound to maintain this level of therapeutic or prophylactic effect over an extended period of time. To maintain a constant level of the compound or a pharmaceutically acceptable salt of the compound in the body, the compound or a pharmaceutically acceptable salt of the compound can be released from the dosage form at a rate that will replace the amount of the compound or a pharmaceutically acceptable salt of the compound being metabolized and excreted from the body. Controlled or sustained release of the compound or pharmaceutically acceptable salt of the compound can be stimulated by various conditions, including but not limited to, changes in pH, changes in temperature, concentration or availability of enzymes that can metabolize the formulation, concentration or availability of water, or other physiological conditions or compounds.

In certain embodiments, the present invention is directed to prodrugs of the compounds or pharmaceutically acceptable salts of compounds described herein. Various forms of prodrugs are known in the art, for example as discussed in Bundgaard (ed.), Design of Prodrugs, Elsevier (1985); Widder et al. (ed.), Methods in Enzymology, Vol. 4, Academic Press (1985); Kgrogsgaard-Larsen et al. (ed.); “Design and Application of Prodrugs”, Textbook of Drug Design and Development, Chapter 5, pp. 113-191 (1991); Bundgaard et al., Journal of Drug Delivery Reviews, 8:1-38 (1992); Bundgaard et al., J. Pharmaceutical Sciences, 77:285 et seq. (1988); and Higuchi and Stella (eds.), Prodrugs as Novel Drug Delivery Systems, American Chemical Society (1975).

### Dosage

In vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a healthcare practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a healthcare practitioner. The effective dosage amounts described herein refer to total amounts administered; that is, if more than one compound or a pharmaceutically acceptable salt of the compound is administered, the effective dosage amounts correspond to the total amount administered.

The amount of the compound or a pharmaceutically acceptable salt of the compound that is effective for treating or preventing a disorder (e.g., a nervous system disorder) will typically range from about 0.001 mg/kg to about 600 mg/kg of body weight per day, in certain embodiments, from about 1 mg/kg to about 600 mg/kg body weight per day, in one embodiment, from about 1 mg/kg to about 250 mg/kg body weight per day, in another embodiment, from about 10 mg/kg to about 400 mg/kg body weight per day, in another embodiment, from about 10 mg/kg to about 200 mg/kg of body weight per day, in another embodiment, from about 10 mg/kg to about 100 mg/kg of body weight per day, in one embodiment, from about 10 mg/kg to about 25 mg/kg body weight per day, in another embodiment, from about 1 mg/kg to about 10 mg/kg body weight per day, in another embodiment, from about 0.001 mg/kg to about 100 mg/kg of body weight per day, in another embodiment, from about 0.001 mg/kg to about 10 mg/kg of body weight per day, and in another embodiment, from about 0.001 mg/kg to about 1 mg/kg of body weight per day.

In certain embodiments, the pharmaceutical composition is in unit dosage form, e.g., as a tablet, capsule, powder, solution, suspension, emulsion, granule, or suppository. In such form, the composition is sub-divided in unit dose containing appropriate quantities of the compound or pharmaceutically acceptable salt of the compound; the unit dosage form can be packaged compositions, for example, packeted powders, vials, ampoules, prefilled syringes or sachets containing liquids. The unit dosage form can be, for example, a capsule or tablet itself, or it can be the appropriate number of any such compositions in package form. Such unit dosage form may contain sufficient compound to administer from about 0.01 mg/kg to about 250 mg/kg, in some embodiments, from about 1 mg/kg to about 250 mg/kg, in other embodiments from about 10 mg/kg to about 25 mg/kg and may be given in a single dose or in two or more divided doses. Variations in the dosage will necessarily occur depending upon the species, weight and condition of the patient being treated and the patient's individual response to the medicament.

In some embodiments, the unit dosage form comprises about 0.01 to about 1000 mg of a compound described herein. In other embodiments, the unit dosage form is about 0.01 to about 500 mg; in another embodiment, the unit dosage form is about 0.01 to about 250 mg; in another embodiment, the unit dosage form is about 0.01 to about 100 mg; in another embodiment, the unit dosage form is about 0.01 to about 50 mg; in another embodiment, the unit dosage form is about 0.01 to about 25 mg; in another embodiment, the unit dosage form is about 0.01 to about 10 mg; in another embodiment, the unit dosage form is about 0.01 to about 5 mg; and in another embodiment, the unit dosage form is about 0.01 to about 10 mg.

### Treatment

The compound or a pharmaceutically acceptable salt of the compound can be assayed in vitro or in vivo for the desired therapeutic or prophylactic activity prior to use in humans. Animal model systems can be used to demonstrate safety and efficacy. In some cases, the amount of a compound or a pharmaceutically acceptable salt of the compound delivered is an amount that is effective for treating (e.g., ameliorating) or preventing a central nervous system disorder.

The present methods for treating or preventing a central nervous system disorder can further comprise administering at least one additional therapeutic agent to the animal being administered the compound or a pharmaceutically acceptable salt of the compound. Typically, the additional therapeutic agent is administered in an effective amount, e.g., to ameliorate at least one symptom of a disorder being treated or to increase the efficacy of a compound or the invention.

Effective amounts of the other therapeutic agents are known to those skilled in the art. However, it is well within the skilled artisan's purview to determine the other therapeutic agent's optimal effective amount range. The compound or a pharmaceutically acceptable salt of the compound and the additional therapeutic agent can act additively or, in some embodiments, synergistically. In certain embodiments, where another therapeutic agent is administered to an animal, the effective amount of the compound or a pharmaceutically acceptable salt of the compound is less than its effective amount would be where the other therapeutic agent is not administered. In this case, without being bound by theory, it is believed that the compound or a pharmaceutically acceptable salt of the compound and the other therapeutic agent act synergistically. In some cases, the patient in need of treatment is being treated with one or more other therapeutic agents. In some cases, the patient in need of treatment is being treated with at least two other therapeutic agents. In some embodiments, the additional therapeutic agent is selected from the group consisting of one or more anti-depressant agents (e.g., an SSRI, monoamine oxidase inhibitor, norepinephrine reuptake inhibitor, or serotonin and noradrenaline reuptake inhibitor), anti-anxiety agent (e.g., benzodiazepine, serotonin 1A (5-HT_{1A}) agonist a (5-HT_{1A}) antagonist (such as 5-HT_{1A} partial agonists), or corticotrophin releasing factor), an anti-psychotic agent (e.g., phethiazine, piperainze phenothiazine, butyrophenone, substituted benzamide, thioxanthine, haloperidol, olanzapine, clozapine, risperidone, pimozide, aripiprazol, or ziprasidone), or a cognitive enhancer (e.g., an acetylcholinesterase inhibitor, a cholinesterase inhibitor, cholinergic receptor agonist, or serotonin receptor antagonist, a drug that can modulate the level of soluble Aβ amyloid fibril formation, a drug that can modulate the amyloid plaque burden, or a drug that can protect neuronal activity).

In certain embodiments, the compound or a pharmaceutically acceptable salt of the compound is administered concurrently with another therapeutic agent.

In some embodiments, a composition comprising an effective amount of the compound or a pharmaceutically acceptable salt of the compound and an effective amount of another therapeutic agent within the same composition can be administered.

In other embodiments, a composition comprising an effective amount of the compound or a pharmaceutically acceptable salt of the compound and a separate composition comprising an effective amount of another therapeutic agent can be concurrently administered. In another embodiment, an effective amount of the compound or a pharmaceutically acceptable salt of the compound is administered prior to or subsequent to administration of an effective amount of another therapeutic agent. In this embodiment, the compound or a pharmaceutically acceptable salt of the compound is administered while the other therapeutic agent exerts its therapeutic effect, or the other therapeutic agent is administered while the compound or a pharmaceutically acceptable salt of the compound exerts its preventative or therapeutic effect for treating or preventing a central nervous system disorder.

Thus, in some cases, a composition comprising an effective amount of the compound or a pharmaceutically acceptable salt of the compound of the present invention and a pharmaceutically acceptable carrier is provided. In another embodiment, the composition further comprises a second therapeutic agent. In some embodiments, the second therapeutic agent includes one or more other antidepressants, anti-anxiety agents, anti-psychotic agents or cognitive enhancers. In another embodiment, the pharmaceutically acceptable carrier is suitable for oral administration and the composition comprises an oral dosage form.

In certain embodiments, the compounds or pharmaceutically acceptable salts of the compounds described herein are useful as modulators of the activity of a 5-HT_{1A} receptor. For example, in some cases, the compounds or pharmaceutically acceptable salts of the compounds described herein bind to a 5-HT_{1A}. In certain embodiments, a compound or pharmaceutically acceptable salt of a compound described herein is useful as 5-HT_{1A} receptor antagonist or as 5-HT_{1A} agonist or partial agonist. Compounds that modulate the activity of 5-HT_{1A} receptors, for example by binding to a 5-HT_{1A} or acting as agonists and/or antagonists at the receptor, can be readily identified by those skilled in the art using art-recognized methods, including pharmacological test procedures such as those described herein. Accordingly, in some embodiments, the compounds and pharmaceutically acceptable salts of the compounds described herein are useful for treating a mammal with a central nervous system disorder that is mediated, directly or indirectly, through the 5-HT_{1A} pathway. Central nervous system disorders include, without limitation, anxiety-related disorders (e.g., buspirone (Buspar®) a 5-HT_{1A} partial agonist is marketed as an anxiolytic), cognition-related disorders (Menese and Perez-Garcia, Neuroscience & Biobehavioral Reviews 31:705-727, (2007)); depression and depression-related disorders (Artegas et al., Current Drug Targets 7:139-147 (2006)), and schizophrenia and other psychotic disorders. Thus, in some embodiments, the compounds and pharmaceutically acceptable salts of the compounds described herein that act as 5-HT_{1A} receptor modulators are useful for treating a mammal with a cognition-related disorder, an anxiety-related disorder, depression or schizophrenia.

Exemplary cognition-related disorders (e.g., cognitive dysfunction) include, without limitation, mild cognitive impairment (MCI), dementia, delirium, amnestic disorder, Alzheimer's disease, Parkinson's disease, Huntington's disease; memory disorders including memory deficits associated with depression, senile dementia, dementia of Alzheimer's disease; cognitive deficits or cognitive dysfunction associated with neurological conditions including, for example, Parkinson's disease (PD), Huntington's disease (HD), Alzheimer's disease, depression, schizophrenia and other psychotic disorders such as paranoia and manic-depressive illness; cognitive dysfunction in schizophrenia; disorders of attention and learning such as attention deficit disorders (e.g., attention deficit hyperactivity disorder (ADHD)) and dyslexia; cognitive dysfunction associated with developmental disorders such as Down's syndrome and Fragile X syndrome; loss of executive function; loss of learned information; vascular dementia; schizophrenia; cognitive decline; a neurodegenerative disorder; and other dementias, for example, dementia due to HIV disease, head trauma, Parkinson's disease, Huntington's disease, Pick's disease, Creutzfeldt-Jakob disease, or due to multiple etiologies. Cognition-related disorders also include, without limitation, cognitive dysfunction associated with MCI and dementias such as Lewy Body, vascular, and post stroke dementias. Cognitive dysfunction associated with surgical procedures, traumatic brain injury or stroke may also be treated in accordance with the embodiments described herein.

Exemplary anxiety-related disorders include, without limitation, generalized anxiety disorder, attention deficit disorder, attention deficit hyperactivity disorder, obsessive compulsive disorder, substance addiction, withdrawal from drug, alcohol or nicotine addiction, panic disorder, panic attacks, post traumatic stress disorder, premenstrual dysphoric disorder, social anxiety disorder, eating disorders such as anorexia nervosa and bulimia nervosa, vasomotor flushing, and phobias including social phobia, agoraphobia, and specific phobias. Substance addiction includes, without limitation, drug, alcohol or nicotine addiction.

In some embodiments, the compounds or pharmaceutically acceptable salts of the compounds described herein are useful as modulators of serotonin reuptake. For example, in some embodiments, the compounds or pharmaceutically acceptable salts of the compounds described herein can block the reuptake of the brain neurotransmitter serotonin. Accordingly, the compounds or pharmaceutically acceptable salts of the compounds described herein are useful for the treatment or prevention of conditions that can be treated by the administration of a serotonin selective reuptake inhibitor (SSRI), such as depression (including but not limited to major depressive disorder, childhood depression and dysthymia), anxiety, panic disorder, post-traumatic stress disorder, premenstrual dysphoric disorder (e.g., pre-menstrual syndrome), attention deficit disorder (with and without hyperactivity), obsessive compulsive disorders (including but not limited to trichotillomania), obsessive compulsive spectrum disorders (including but not limited to autism), social anxiety disorder, generalized anxiety disorder, obesity, eating disorders such as anorexia nervosa, bulimia nervosa, vasomotor flushing, cocaine and alcohol addiction, sexual dysfunction (including but not limited to premature ejaculation), incontinence (including, but not limited to fecal incontinence, urge incontinence, overflow incontinence, passive incontinence, reflex incontinence, stress urinary incontinence urinary exertional incontinence and urinary incontinence), and pain (including, but not limited to migraine, chronic back pain, phantom limb pain, neuropathic pain such as diabetic neuropathy, and post herpetic neuropathy) and related illnesses.

The compounds or pharmaceutically acceptable salts of the compounds described herein are also useful for the treatment or prevention of conditions mediated through 5-HT_{1A} receptors (5-HT_{1A}-related disorders) (e.g., conditions that can be treated by the administration of a 5-HT_{1A} agonist or antagonist or for which 5-HT_{1A} receptor is involved in expression of one or more symptoms of the condition), such as depression, such as single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder, pediatric depression, child abuse induced depression and postpartum depression; bipolar disorders or manic depression, for example, bipolar I disorder, bipolar II disorder and cyclothymic disorder; conduct disorder; disruptive behavior disorder; disorders of attention and learning such as attention deficit hyperactivity disorder (ADHD) and dyslexia; behavioral disturbances associated with mental retardation, autistic disorder, pervasive development disorder and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia, substance-induced psychotic disorder, shared psychotic disorder, and psychotic disorder due to a general medical condition; delirium, dementia, and amnestic and other cognitive or neurodegenerative disorders, such as Parkinson's disease (PD), Huntington's disease (HD), Alzheimer's disease, senile dementia, dementia of the Alzheimer's type, mild cognitive impairment (MCI), memory disorders, loss of executive function, vascular dementia, and other dementias, for example, due to HIV disease, head trauma, Parkinson's disease, Huntington's disease, Pick's disease, Creutzfeldt Jakob disease, or due to multiple etiologies; cognitive deficits associated with neurological conditions including, for example, Parkinson's disease (PD), Huntington's disease (HD), Alzheimer's disease; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremor; chemical dependencies and addictions (e.g., dependency on, or addiction to, alcohol, heroin, cocaine, benzodiazepines, nicotine, or phenobarbital); behavioral addictions such as an addiction to gambling; and ocular disorders such as glaucoma and ischemic retinopathy; sexual dysfunction associated with drug treatment (e.g., sexual dysfunction associated with SSRIs).

In some embodiments, the compounds and pharmaceutically acceptable salts of the compounds described herein have dual-acting mechanisms. That is, the compounds and pharmaceutically acceptable salts of the compounds can modulate serotonin reuptake and can modulate 5-HT_{1A} receptor activity (e.g., through binding and/or agonism or antagonism).

In certain embodiments, a method for modulating the activity of a 5-HT_{1A} receptor is provided. The method includes contacting a 5-HT_{1A} receptor with one or more compounds or pharmaceutically acceptable salts of the compounds described herein. In some embodiments, a method of binding a 5-HT_{1A} receptor in a patient is provided. The method includes administering to the patient an effective amount of one or more compounds or pharmaceutically acceptable salts of the compounds described herein. In certain embodiments, a method of antagonizing a 5-HT_{1A} receptor is provided. In other embodiments, a method of agonizing a 5-HT_{1A} receptor is provided. Such methods include administering an effective amount of one or more compounds or pharmaceutically acceptable salts of the compounds described herein. In some embodiments, the method includes administration to a patient suffering from a 5-HT_{1A}-related disorder. In certain embodiments, a method of modulating serotonin reuptake in a patient is provided. The method includes administering an effective amount of one or more compounds or pharmaceutically acceptable salts of the compounds described herein.

In some embodiments, a method for treating depression, comprising administering to a mammal in need thereof a compound or a pharmaceutically acceptable salt of a compound or pharmaceutically acceptable salt of a compound described herein in an amount effective to treat depression is provided. In some cases, the method for treating depression includes administering a second therapeutic agent. In some embodiments, the second therapeutic agent is an anti-depressant agent, an anti-anxiety agent, an anti-psychotic agent, or a cognitive enhancer.

In some embodiments, pharmaceutical compositions or medicaments are provided. The pharmaceutical compositions or medicaments include one or more compounds or pharmaceutically acceptable salts of the compounds described herein. In some embodiments, the pharmaceutical compositions also include one or more pharmaceutically acceptable carriers. In some embodiments, the pharmaceutical composition is useful for modulating the activity of a 5-HT_{1A} receptor (e.g., by binding and agonism or antagonism of the receptor). In some embodiments, the pharmaceutical composition is useful for modulating serotonin reuptake in a patient. In some embodiments, the pharmaceutical composition is useful for treating a central nervous system disorder. In some embodiments, a pharmaceutical composition for treating depression is provided. The composition includes a compound or a pharmaceutically acceptable salt of a compound described herein.

In some embodiments, the compounds or pharmaceutically acceptable salts of the compounds described herein demonstrate fewer undesired side effects than observed for other central nervous system treatments. Typically, the compound or pharmaceutically acceptable salt of the compound, when administered in an effective amount, exhibits fewer side effects or at least one less severe side effect compared to a known treatment (e.g., a specific drug) for at lease one disorder specified herein. For example, a side effect associated with some antidepressant therapy (such as SSRIs) is sexual dysfunction. In some embodiments, administration of a compound or pharmaceutically acceptable salt of a compound described herein demonstrates a lower incidence or degree of sexual dysfunction than that associated with at least one SSRI, tricyclic antidepressants, aminoketone class compound, monoamine oxidase inhibitor (MAOI), serotonin and norepinephrine reuptake inhibitor (SNRI), norepinephrine reuptake inhibitor (NRI), partial 5-HT_{1A} agonist, 5-HT_{2A} receptor antagonist, or antipsychotic drug (typical and atypical). Accordingly, in certain embodiments, the compounds or pharmaceutically acceptable salts of the compounds described herein are useful for treating sexual dysfunction, e.g., sexual dysfunction associated with drug treatment such as drug treatment with an antidepressant, an antipsychotic, or an anticonvulsant. In some embodiments, the sexual dysfunction comprises a deficiency in penile erection.

The compounds and pharmaceutically acceptable salts of the compounds described herein are also useful in the manufacture of medicaments for treating a central nervous system disorder in a mammal. Similarly, the compounds and pharmaceutically acceptable salts of the compounds described herein are also useful in the manufacture of medicaments for treating a cognition-related disorder, an anxiety-related disorder, depression, or schizophrenia in a mammal. Also, the compounds and pharmaceutically acceptable salts of the compounds of described herein are useful in the manufacture of medicaments for modulating the activity of a 5-HT_{1A} receptor in a mammal. In some embodiments, the compounds and pharmaceutically acceptable salts of the compounds of described herein are also useful in the manufacture of medicaments for modulating serotonin reuptake in a mammal.

The invention is further illustrated by the following examples, which include non-limiting examples for production of representative compounds disclosed herein. The examples are provided for illustrative purposes only. They are not to be construed as limiting the scope or content of the invention in any way.

### EXAMPLES

### Example 1

### 3-(3,4-dihydroquinolin-1(2H)-yl)-N-{[(2S)-8-methyl-2,3-dihydro[1,4]dioxino[2,3-f]quinolin-2-yl]methyl}propan-1-amine

1a: 3-({[(2S)-8-methyl-2,3-dihydro[1,4]dioxino[2,3-f]quinolin-2-yl]methyl}amino)propan-1-ol

[(2R)-8-methyl-2,3-dihydro[1,4]dioxino[2,3-f]quinolin-2-yl]methyl 4-bromobenzene-sulfonate 1.50 g, 3.33 mmol) was dissolved in DMSO (3 mL) and then stirred with 3-amino-1-propanol (1.3 mL, 16.7 mmol) and Et₃N (1.39 mL, 10.0 mmol). This reaction mixture was then irradiated at about 150° C. for approximately 30 minutes using a microwave. Thin layer chromatography (TLC) indicated reaction to be complete. The resulting material was then diluted with CH₂Cl₂, washed with H₂O followed by brine, dried over Na₂SO₄, and concentrated under reduced pressure. The crude product was purified by silica gel chromatography to afford the product as a light brown oil (0.80 g, 83%); MS (ES) m/z=289.1 [M+H]+, 311.1 [M+Na]+.

1b: Tert-butyl (3-hydroxypropyl){[(2S)-8-methyl-2,3-dihydro[1,4]dioxino[2,3-f]quinolin-2-yl]methyl}carbamate

3-({[(2S)-8-methyl-2,3-dihydro[1,4]dioxino[2,3-f]quinolin-2-yl]methyl}amino)propan-1-ol (1a) (0.80 g, 2.8 mmol) was dissolved in CH₂Cl₂ (10 mL) and then treated with di-tert-butyl dicarbonate (0.91 g, 4.2 mmol). After approximately 0.5 hours, TLC indicated the reaction was complete. The reaction mixture was concentrated and purified by silica gel chromatography to afford the product as a near colorless oil (0.90 g, 84%); MS (ES) m/z=389.2 [M+H]+, 411.2 [M+Na]+, 799.4 [2M+Na]+.

1c: (8-Methyl-2,3-dihydro-[114]dioxino[2,3-f]quinolin-2-yl-methyl)-(3-oxo-propyl)-carbamic acid tert-butyl ester

Tert-butyl (3-hydroxypropyl) {[(2S)-8-methyl-2,3-dihydro[1,4]dioxino[2,3-f]quinolin-2-yl]methyl}carbamate (1b) (0.90 g, 2.3 mmol) was dissolved in CH₂Cl₂ (25 mL) and cooled to approximately 0° C. before being treated with Dess-Martin Periodinane (0.983 g, 2.3 mmol). After about 2 hours, TLC indicated that the reaction was complete. The reaction mixture was diluted with Et₂O (50 mL) and was then poured into a stirring solution of Na₂S₂O₃ (2.00 g) in NaHCO₃ (100 mL) and was stirred until both phases became clear. The organic layer was separated, dried over Na₂SO₄, and concentrated to afford product as a light brown oil and was used without purification (0.70 g, 78%); LC/MS m/z=387.2 [M+H]+.

1d: Tert-butyl [3-(3,4-dihydroquinolin-1(2H)-yl)-propyl]{[(2S)-8-methyl-2,3-dihydro[1,4]dioxino[2,3-f]quinolin-2-yl]methyl}carbamate

(8-Methyl-2,3-dihydro-[1,4]dioxino[2,3-f]quinolin-2-yl)-(3-oxo-propyl)-carbamic acid tert-butyl ester (1c) (0.05 g) and tetrahydroquinoline (0.03 g) were dissolved in MeOH (1 mL) and then treated with NaCNBH₃ (0.02 g) and AcOH (glacial; 0.1 mL). After about 18 hours, the reaction mixture was carefully basified with 2.5 M NaOH until pH˜12. The reaction mixture was extracted with CH₂Cl₂ (2×). The organic layer was washed with brine, dried over Na₂SO₄, concentrated, and purified by silica gel chromatography to afford product as a near colorless oil.

1e: 3-(3,4-dihydroquinolin-1(2H)-yl)-N-{[(2S)-8-methyl-2,3-dihydro[1,4]dioxino[2,3-f]quinolin-2-yl]methyl}propan-1-amine

Tert-butyl [3-(3,4-dihydroquinolin-1(2H)-yl)propyl]{[(2S)-8-methyl-2,3-dihydro[1,4]dioxino[2,3-f]quinolin-2-yl]methyl}carbamate (1d) was dissolved in CH₂Cl₂ (10 mL) and treated with trifluoroacetic acid (TFA) (0.5 mL). After stirring overnight, reaction mixture was diluted with CH₂Cl₂, washed with NaHCO₃ followed by brine. The organic layer was dried over Na₂SO₄, concentrated and purified by silica gel chromatography to afford the desired amine. The amine was converted into the hydrochloride salt by treating a solution of the amine in CH₂Cl₂ with 1N HCL in ether. The resulting yellow solid was collected by evaporation of the solvent under a stream of nitrogen to afford the title product as the hydrochloride salt (0.05 g) as yellow solid; MS (ES) m/z=404.2 [M+H]+.

### Example 2

### 3-(5-fluoro-2,3-dihydro-1H-indol-1-yl)-N-{[(2S)-8-methyl-2,3-dihydro[1,4]dioxino[2,3-f]quinolin-2-yl]methyl}propan-1-amine

2a: Tert-butyl [3-(5-fluoro-2,3-dihydro-1H-indol-1-yl)propyl]{[(2S)-8-methyl-2,3-dihydro[1,4]dioxino[2,3-f]quinolin-2-yl]methyl}carbamate

(8-Methyl-2,3-dihydro-[1,4]dioxino[2,3-f]quinolin-2-yl)-(3-oxo-propyl)-carbamic acid tert-butyl ester (1c) (0.13 g, 0.34 mmol) and 5-fluoroindoline (0.19 g, 1.4 mmol) were dissolved in MeOH and then treated with NaCNBH₃ (0.19 g, 3.0 mmol) and AcOH (glacial; 0.2 mL). After approximately 2 hours, TLC indicated reaction was complete. The reaction mixture was carefully basified with 2.5M NaOH until pH˜12. Then, the reaction mixture was extracted with EtOAc. The organic layer was washed with brine, dried over Na₂SO₄, concentrated, and purified by silica gel chromatography to afford product as a near colorless oil (0.130 g, 76%); MS (ES) m/z=508.3 [M+H]+.

2b: 3-(5-fluoro-2,3-dihydro-1H-indol-1-yl)-N-{[(2S)-8-methyl-2,3-dihydro[1,4]dioxino[2,3-f]quinolin-2-yl]methyl}propan-1-amine

Tert-butyl [3-(5-fluoro-2,3-dihydro-1H-indol-1-yl)propyl]{[(2S)-8-methyl-2,3-dihydro[1,4]dioxino[2,3-f]quinolin-2-yl]methyl}carbamate (2a) (0.12 g, 0.24 mmol) was dissolved in CH₂Cl₂ (5 mL) and treated with TFA (1 mL). After stirring overnight, reaction appeared to be complete by TLC. The reaction mixture was diluted with CH₂Cl₂ and washed with NaHCO₃ followed by brine. The organic layer was dried over Na₂SO₄, concentrated and purified by silica gel chromatography to afford the desired amine. The amine was converted into the hydrochloride salt by treating a solution of the amine in CH₂Cl₂ with 1N HCL in ether. The resulting yellow solid was collected by vacuum filtration, washed with ethyl acetate and hexane, and dried under vacuum to afford the title product as the hydrochloride salt (0.047 g); MS (ES) m/z=408.2 [M+H]+.

### Example 3

### 3-(6-fluoro-2,3-dihydro-1H-indol-1-yl)-N-{[(2S)-8-methyl-2,3-dihydro[1,4]dioxino[2,3-f]quinolin-2-yl]methyl}propan-1-amine

3a: Tert-butyl [3-(6-fluoro-2,3-dihydro-1H-indol-1-yl)propyl]{[(2S)-8-methyl-2,3-dihydro[1,4]dioxino[2,3-f]quinolin-2-yl]methyl}carbamate

(8-Methyl-2,3-dihydro-[1,4]dioxino[2,3-f]quinolin-2-yl)-(3-oxo-propyl)-carbamic acid tert-butyl ester (1c) (0.070 g, 0.18 mmol) and 6-fluoroindoline (0.100 g, 0.73 mmol) were reacted as previously described (step 2a) to provide the product as a colorless oil (0.055 g, 60%); MS (ES) m/z=508.2 [M+H]+.

3b: 3-(6-Fluoro-2,3-dihydro-1H-indol-1-yl)-N-{[(2S)-8-methyl-2,3-dihydro[1,4]dioxino[2,3-f]quinolin-2-yl]methyl}propan-1-amine

Tert-butyl [3-(6-fluoro-2,3-dihydro-1H-indol-1-yl)propyl]{[(2S)-8-methyl-2,3-dihydro[1,4]dioxino[2,3-f]quinolin-2-yl]methyl}carbamate (3a) (0.055 g, 0.11 mmol) was converted to the title amine as the hydrochloride salt (0.029 g); MS (ES) m/z=408.2 [M+H]+ according to a procedure similar to Step 2b, previously described.

### Example 4

### 3-(7-fluoro-3,4-dihydroquinolin-1(2H)-yl)-N-{[(2S)-8-methyl-2,3-dihydro[1,4]dioxino[2,3-f]quinolin-2-yl]methyl}propan-1-amine

4a: 7-Fluoro-3,4-dihydroquinolin-2(1H)-one

3-Fluoroaniline (5 mL, 52.02 mmol) was dissolved in acetone (250 mL) and was then treated with pyridine (10.5 mL, 130.1 mmol) and 3-chloropropionyl chloride (6 mL, 62.2 mmol). The reaction mixture was heated to approximately 55° C. and, after about 2 hours, reaction appeared complete by TLC analysis. The reaction mixture was cooled to room temperature, diluted with H₂O, and extracted with EtOAc. The organic layer was dried over Na₂SO₄ and concentrated to afford the amide as an off-white solid, which was used without purification. The amide (2.00 g, 9.9 mmol) and AlCl₃ (4.62 g, 34.7 mmol) were placed together in a 3-neck flask under N₂. Using a mechanical stirrer, this reaction was heated and stirred at 120° C. for 3 hours. TLC indicated reaction was complete. The reaction was cooled to room temperature, and the reaction mixture was treated with 35% HCl (10 mL) and ice (10.00 g). The mixture was stirred until all unreacted AlCl₃ was quenched, after which, the product was filtered and washed with H₂O several times. The resulting product was recrystallized from EtOH to afford the product as an off-white solid (1.25 g, 73% over 2 steps); MS (APPI) m/z=166 [M+H]+.

4b: 7-fluoro-1,2,3,4-tetrahydroquinoline

7-Fluoro-3,4-dihydroquinolin-2(1H)-one (4a) (0.70 g, 4.2 mmol) was stirred together with a BH₃, THF solution (1.0M; 42 mL, 42.0 mmol). The solution was brought to a gentle reflux (˜60° C.). The reaction was allowed to stir at reflux overnight, after which TLC indicated reaction was complete. The mixture was cooled to room temperature, and was carefully treated with MeOH until excess BH₃ was quenched. The reaction mixture was then concentrated to dryness, and residual oil was re-dissolved in MeOH (˜10 mL) and treated with concentrated HCl (˜2 mL). This mixture was allowed to stir at 60° C. for 1 hour. The mixture was then cooled to 0° C. and carefully basified until pH˜12. The mixture was extracted with EtOAc, the organic layer was washed with brine, concentrated and purified by silica gel chromatography to afford product as an off-white solid (0.40 g, 63%); MS (APPI) m/z=152 [M+H]+.

4c: Tert-butyl [3-(7-fluoro-3,4-dihydroquinolin-1(2H)-yl)propyl]{[(2S)-8-methyl-2,3-dihydro[1,4]dioxino[2,3-f]quinolin-2-yl]methyl}carbamate

(8-Methyl-2,3-dihydro-[1,4]dioxino[2,3-f]quinolin-2-yl)-(3-oxo-propyl)-carbamic acid tert-butyl ester (1c) (0.160 g, 0.42 mmol) and 7-fluoro-1,2,3,4-tetrahydroquinoline (4b) (0.25 g, 1.7 mmol) were reacted according to a procedure similar to Step 2a, previously described, to provide the product as a colorless oil (0.11 g, 50%); MS (ES) m/z=522.2 [M+H]+.

4d: 3-(7-fluoro-3,4-dihydroquinolin-1(2H)-yl)-N-{[(2S)-8-methyl-2,3-dihydro[1,4]dioxino[2,3-f]quinolin-2-yl]methyl}propan-1-amine

Tert-butyl [3-(7-fluoro-3,4-dihydroquinolin-1(2H)-yl)propyl]{[(2S)-8-methyl-2,3-dihydro[1,4]dioxino[2,3-f]quinolin-2-yl]methyl}carbamate (4c) (0.11 g, 0.21 mmol) was treated according to a procedure similar to Step 2b, previously described, to provide the amine as the hydrochloride salt (0.067 g); MS (ES) m/z=422.0 [M+H]+.

### Example 5

### 3-(2,3-dihydro-1H-indol-1-yl)-N-{[(2S)-8-methyl-2,3-dihydro[1,4]dioxino[2,3-f]quinolin-2-yl]methyl}propan-1-amine

5a: Tert-butyl [3-(2,3-dihydro-1H-indol-1-yl)propyl]{[(2S)-8-methyl-2,3-dihydro[1.4]dioxino[2,3-f]quinolin-2-yl]methyl}carbamate

A procedure similar to that of Step 1d, previously described, using indoline in place of tetrahydroquinoline provided the product as a colorless oil.

5b: 3-(2,3-dihydro-1H-indol-1-yl)-N-{[(2S)-8-methyl-2,3-dihydro[1,4]dioxino[2,3-f]quinolin-2-yl]methyl}propan-1-amine

A procedure similar to that of Step 1e, previously described, using tert-butyl [3-(2,3-dihydro-1H-indol-1-yl)propyl]{[(2S)-8-methyl-2,3-dihydro[1,4]dioxino[2,3-f]quinolin-2-yl]methyl} (5a) provided the product as the hydrochloride salt; MS (ES) m/z=390.2 [M+H]+.

### Example 6

### 3-(2,3-dihydro-4H-1,4-benzoxazin-4-yl)-N-{[(2S)-8-methyl-2,3-dihydro[1,4]dioxino[2,3-f]quinolin-2-yl]methyl}propan-1-amine

6a: 3,4-dihydro-2H-1,4-benzoxazine

A procedure similar to that of Step 4b, previously described, using 2H-1,4-benzoxazine-3(4H)-one (0.45 g, 3.0 mmol) in place of 7-fluoro-3,4-dihydroquinolin-2(1H)-one provided the product as a colorless oil (0.245 g, 60%); MS (APPI) m/z=136 [M+H]+, 177 {M+ACN+H]+.

6b: 3-(2,3-dihydro-4H-1,4-benzoxazin-4-yl)-N-{[(2S)-8-methyl-2,3-dihydro[1,4]dioxino[2,3-f]quinolin-2-yl]methyl}propan-1-amine

(8-Methyl-2,3-dihydro-[1,4]dioxino[2,3-f]quinolin-2-yl)-(3-oxo-propyl)-carbamic acid tert-butyl ester (1c) (0.10 g, 0.26 mmol) and 3,4-dihydro-2H-1,4-benzoxazine (6a) (0.14 g, 1.0 mmol) were dissolved in MeOH (5 mL) and then treated with NaCNBH₃ (0.15 g, 2.33 mmol) and AcOH (glacial; 0.2 mL). The mixture stirred overnight, after which, TLC indicated the reaction was complete. The mixture was carefully basified with 2.5 M NaOH until pH˜12, then the reaction mixture was extracted with EtOAc. The organic layer was washed with brine, dried over Na₂SO₄, concentrated, and purified by silica gel chromatography to afford product. The product was then dissolved in CH₂Cl₂ (5 mL) and treated with TFA (1 mL). After stirring for approximately 1 hour, the reaction appeared complete by TLC. The reaction mixture was diluted with CH₂Cl₂, washed with NaHCO₃ followed by brine. The organic layer was dried over Na₂SO₄, concentrated and purified by silica gel chromatography to afford the desired amine. The amine was converted into the hydrochloride salt by treating a solution of the amine in CH₂Cl₂ with 1N HCL in ether. The resulting yellow solid was collected by vacuum filtration, washed with ethyl acetate and hexane, and dried under vacuum to afford the title product (0.017 g); MS (ES) m/z=406.2 [M+H]+.

### Example 7

### 3-(6,7-difluoro-3,4-dihydroquinolin-1(2H)-yl)-N-{[(2S)-8-methyl-2,3-dihydro[1,4]dioxino[2,3-f]quinolin-2-yl]methyl}propan-1-amine

7a: 6,7-difluoro-3,4-dihydroquinolin-2(1H)-one

A procedure similar to that of Step 4a, previously described, using 3,4-difluoroaniline (2.50 ml, 29.03 mmol) in place of 3-fluoroaniline provided the product as a white solid (3.90 g, 85% over two steps); MS (ES) m/z=182.0 [M−H]⁻.

7b: 6,7-difluoro-1,2,3,4-tetrahydroquinoline

A procedure similar to that of Step 4b using 6,7-difluoro-3,4-dihydroquinolin-2(1H)-one (7a) (0.549 g, 3.00 mmol) provided the product as a white solid (0.36 g, 71%); MS (APPI) m/z=170 [M+H]+.

7c: 3-(6,7-difluoro-3,4-dihydroquinolin-1(2H)-yl)-N-{8 (2S)-8-methyl-2,3-dihydro[1,4]dioxino[2,3-f]quinolin-2-yl]methyl}propan-1-amine

A procedure similar to that of Step 6b, previously described, using (8-Methyl-2,3-dihydro-[1,4]dioxino[2,3-f]quinolin-2-yl)-(3-oxo-propyl)-carbamic acid tert-butyl ester (1c) (0.10 g, 0.26 mmol) and 6,7-difluoro-1,2,3,4-tetrahydroquinoline (7b) (0.18 g, 1.04 mmol) in place of 3,4-dihydro-2H-1,4-benzoxazine provided the desired amine as the hydrochloride salt (0.048 g); MS (ES) m/z=440.2 [M+H]+.

### Example 8

### 3-(6-fluoro-3,4-dihydroquinolin-1(2H)-yl)-N-{[(2S)-8-methyl-2,3-dihydro[1,4]dioxino[2,3-f]quinolin-2-yl]methyl}propan-1-amine

8a: 6-Fluoro-3,4-dihydro-1H-quinolin-2-one

A procedure similar to that of 4a, previously described, using 4-fluoroaniline (2.50 ml, 26.39 mmol) in place of 3-fluoroaniline provided the product as a light brown solid (3.20 g, 74% over two steps); MS (EI) m/z=165 [M+H]+.

8b: 6-fluoro-1,2,3,4-tetrahydroquinoline

A procedure similar to that of Step 4b, previously described, using 6-fluoro-3,4-dihydro-1H-quinolin-2-one (8a) (0.50 g, 3.00 mmol) provided the product as a clear solid (0.3 g, 68%); MS (APPI) m/z=152 [M+H]+.

8c: 3-(6-fluoro-3,4-dihydroquinolin-1(2H)-yl)-N-{[(2S)-8-methyl-2,3-dihydro[1,4]dioxino[2,3-f]quinolin-2-yl]methyl}propan-1-amine

A procedure similar to that of Step 6b, previously described, using (8-methyl-2,3-dihydro-[1,4]dioxino[2,3-f]quinolin-2-yl)-(3-oxo-propyl)-carbamic acid tert-butyl ester (1c) (0.10 g, 0.26 mmol) and 6-fluoro-1,2,3,4-tetrahydroquinoline (0.16 g, 1.04 mmol) (8b) in place of 3,4-dihydro-2H-1,4-benzoxazine provided the title amine as the hydrochloride salt (0.024 g); MS (ES) m/z=422.2 [M+H]+.

### Example 9

### 3-(6,8-difluoro-3,4-dihydroquinolin-1(2H)-yl)-N-{[(2S)-8-methyl-2,3-dihydro[1,4]dioxino[2,3-f]quinolin-2-yl]methyl}propan-1-amine

9a: 6,8-difluoro-3,4-dihydroquinolin-2(1H)-one

A procedure similar to that of Step 4a, previously described, using 2,4-difluoroaniline (3.00 g, 23.24 mmol) in place of 3-fluoroaniline provided the product as a white solid (3.20 g, 52% over two steps); MS (ES) m/z=182.1 [M−H]−.

9b: 6,8-difluoro-1,2,3,4-tetrahydroquinoline

A procedure similar to that of Step 4b, previously described, using 6,8-difluoro-3,4-dihydroquinolin-2(1H)-one (9a) (0.27 g, 1.45 mmol) provided the product as a clear oil (0.19 g, 78%); MS (APPI) m/z=170 [M+H]+, 211 {M+ACN+H]+.

9c: 3-(6,8-difluoro-3,4-dihydroquinolin-1(2H)-yl)-N-{[(2S)-8-methyl-2,3-dihydro[1,4]dioxino[2,3-f]quinolin-2-yl]methyl}propan-1-amine

A procedure similar to that of Step 6b, previously described, using (8-methyl-2,3-dihydro-[1,4]dioxino[2,3-f]quinolin-2-yl)-(3-oxo-propyl)-carbamic acid tert-butyl ester (1c) (0.10 g, 0.26 mmol) and 6,8-difluoro-1,2,3,4-tetrahydroquinoline (9b) (0.18 g, 1.04 mmol) in place of 3,4-dihydro-2H-1,4-benzoxazine provided the desired amine as the hydrochloride salt (0.020 g); MS (ES) m/z=440.2 [M+H]+.

### Example 10

### 3-(7-chloro-6-fluoro-3,4-dihydroquinolin-1(2H)-yl)-N-{[(2S)-8-methyl-2,3-dihydro[1,4]dioxino[2,3-f]quinolin-2-yl]methyl}propan-1-amine

10a: 7-chloro-6-fluoro-3,4-dihydroquinolin-2(1H)-one

A procedure similar to that of Step 4a, previously described, using 3-chloro-4-fluoroaniline (3.64 g, 25.00 mmol) in place of 3-fluoroaniline provided the product as a white solid (2.01 g, 40% over two steps); MS (ES) m/z=198.0 [M−H]−.

10b: 7-chloro-6-fluoro-1,2,3,4-tetrahydroquinoline

A procedure similar to that of Step 4b, previously described, using 7-chloro-6-fluoro-3,4-dihydroquinolin-2(1H)-one (10a) (0.60 g, 3.00 mmol) provided the product as a white solid (0.37 g, 67%); MS (ES) m/z=186.0 [M+H]+.

10c: 3-(7-chloro-6-fluoro-3,4-dihydroquinolin-1(2H)-yl)-N-{[(2S)-8-methyl-2,3-dihydro[1,4]dioxino[2,3-f]quinolin-2-yl]methyl}-propan-1-amine

A procedure similar to that of 6b, previously described, using (8-Methyl-2,3-dihydro-[1,4]dioxino[2,3-f]quinolin-2-yl)-(3-oxo-propyl)-carbamic acid tert-butyl ester (1c) (0.10 g, 0.26 mmol) and 7-chloro-6-fluoro-1,2,3,4-tetrahydroquinoline (10b) (0.19 g, 1.04 mmol) in place of 3,4-dihydro-2H-1,4-benzoxazine provided the title amine as the hydrochloride salt (0.049 g); MS (ES) m/z=456.1 [M+H]+.

### Example 11

### 3-(6-fluoro-2,3-dihydro-4H-1,4-benzoxazin-4-yl)-N-{[(2S)-8-methyl-2,3-dihydro[1,4]dioxino[2,3-f]quinolin-2-yl]methyl}propan-1-amine

11a: 6-fluoro-3,4-dihydro-2H-1,4-benzoxazine

A procedure similar to that of Step 4b, previously described, using 6-fluoro-2H-1,4-benzoxazin-3(4H)-one (0.50 g, 3.0 mmol) provided the product as a white solid (0.45 g, 98%); MS (APPI) m/z=154 [M+H]+.

11b: 3-(6-fluoro-2,3-dihydro-4H-1,4-benzoxazin-4-yl)-N-{[(2S)-8-methyl-2,3-dihydro[1,4]dioxino[2,3-f]quinolin-2-yl]methyl}propan-1-amine

A procedure similar to that of Step 6b, previously described, using (8-Methyl-2,3-dihydro-[1,4]dioxino[2,3-f]quinolin-2-yl)-(3-oxo-propyl)-carbamic acid tert-butyl ester (1c) (0.10 g, 0.26 mmol) and 6-fluoro-3,4-dihydro-2H-1,4-benzoxazine (11a) (0.16 g, 1.0 mmol) in place of 3,4-dihydro-2H-1,4-benzoxazine provided the title amine as the hydrochloride salt (0.053 g); MS (ES) m/z=424.1 [M+H]+.

### Example 12

### N-{[(2S)-8-methyl-2,3-dihydro[1,4]dioxino[2,3-f]quinolin-2-yl]methyl}-3-(2,3,4,5-tetrahydro-1H-1-benzazepin-1-yl)propan-1-amine

12a: Tert-butyl [3-(2,3,4,5-tetrahydro-1H-1-benzazepin-1-yl)propyl]{[(2S)-8-methyl-2,3-dihydro[1,4]dioxino[2,3-f]quinolin-2-yl]methyl}carbamate

A procedure similar to that of Step 1d, previously described, using benzazepine in place of tetrahydroquinoline provided the product as colorless oil.

12b: N-{[(2S)-8-methyl-2,3-dihydro[1,4]dioxino[2,3-f]quinolin-2-yl]methyl}-3-(2,3,4,5-tetrahydro-1H-1-benzazepin-1-yl)propan-1-amine

A procedure similar to that of Step 1e, previously described, using tert-butyl [3-(2,3,4,5-tetrahydro-1H-1-benzazepin-1-yl)propyl]{[(2S)-8-methyl-2,3-dihydro[1,4]dioxino[2,3-f]quinolin-2-yl]methyl}carbamate (12a) provided the title product as the hydrochloride salt; MS (ES) m/z=418.2 [M+H]+.

### Example 13

### N-(2-chlorophenyl)-N-methyl-N′-{[(2S)-8-methyl-2,3-dihydro[1,4]dioxino[2,3-f]quinolin-2-yl]methyl}propane-1,3-diamine

13a: N-(2-chlorophenyl)-N-methyl-N′-{[(2S)-8-methyl-2,3-dihydro[1,4]dioxino[2,3-f]quinolin-2-yl]methyl}propane-1,3-diamine

A procedure similar to that of 6b, previously described, using (8-methyl-2,3-dihydro-[1,4]dioxino[2,3-f]quinolin-2-yl)-(3-oxo-propyl)-carbamic acid tert-butyl ester (1c) (0.10 g, 0.3 mmol) and 2-chloro-N-methylaniline (0.15 g, 1.0 mmol) in place of 3,4-dihydro-2H-1,4-benzoxazine provided the title amine as the hydrochloride salt (0.036 g); MS (ES) m/z=412.1 [M+H]+.

### Example 14

### 4-(6-fluoro-2,3-dihydro-1H-indol-1-yl)-N-{[(2S)-8-methyl-2,3-dihydro[1,4]dioxino[2,3-f]quinolin-2-yl]methyl}butan-1-amine

14a: 4-({[(2S)-8-methyl-2,3-dihydro[1,4]dioxino[2,3-f]quinolin-2-yl]methyl}amino)butan-1-ol

A procedure similar to that of Step 1a, previously described, using 4-amino-1-propanol (1.50 g, 3.3 mmol) in place of 3-amino-1-propanol provided the product as a colorless oil (0.750 g, 75%); MS (APPI) m/z=303 [M+H]+.

14b: Tert-butyl (4-hydroxybutyl){[(2S)-8-methyl-2,3-dihydro[1,4]dioxino[2,3-f]quinolin-2-yl]methyl}carbamate

A procedure similar to that of Step 1b, previously described, using 4-({[(2S)-8-methyl-2,3-dihydro[1,4]dioxino[2,3-f]quinolin-2-yl]methyl}amino)butan-1-ol (14a) (0.72 g, 2.4 mmol) provided the product as a white solid (0.90 g, 94%); LC/MS m/z=403.2 [M+H]+.

14c: (8-Methyl-2,3-dihydro-[1,4]dioxino[2,3-f]quinolin-2-yl)-(4-oxo-butyl)-carbamic acid tert butyl ester

A procedure similar to that of Step 1c, previously described, using product from tert-butyl (4-hydroxybutyl){[(2S)-8-methyl-2,3-dihydro[1,4]dioxino[2,3-f]quinolin-2-yl]methyl}carbamate (14b) (0.65 g, 1.6 mmol) afforded the product as a colorless oil (0.60 g, 93%); LC/MS m/z=401.2 [M+H]+.

14d: Tert-butyl [4-(6-fluoro-2,3-dihydro-1H-indol-1-yl)butyl]{[(2S)-8-methyl-2,3-dihydro[1,4]dioxino[2,3-f]quinolin-2-yl]methyl}carbamate

A procedure similar to that of Step 2a, previously described, using (8-methyl-2,3-dihydro-[1,4]dioxino[2,3-f]quinolin-2-yl)-(4-oxo-butyl)-carbamic acid tert butyl ester (14c) (0.12 g, 0.3 mmol) and 6-fluoroindoline (0.17 g, 1.2 mmol) in place of 5-fluoroindoline afforded product as a near colorless oil (0.113 g, 70%); MS (ES) m/z=522.3 [M+H]+.

14e: 4-(6-fluoro-2,3-dihydro-1H-indol-1-yl)-N-{[(2S)-8-methyl-2,3-dihydro[1,4]dioxino[2,3-f]quinolin-2-yl]methyl}butan-1-amine

A procedure similar to Step 2b, previously described, using tert-butyl [4-(6-fluoro-2,3-dihydro-1H-indol-1-yl)butyl]{[(2S)-8-methyl-2,3-dihydro[1,4]dioxino[2,3-f]quinolin-2-yl]methyl}carbamate (14d) (0.113 g, 0.2 mmol) provided the desired amine as the hydrochloride salt (0.096 g); MS (ES) m/z=422.2 [M+H]+.

### Example 15

### 4-(3,4-dihydroquinolin-1(2H)-yl)-N-{[(2S)-8-methyl-2,3-dihydro[1,4]dioxino[2,3-f]quinolin-2-yl]methyl}butan-1-amine

15a: Tert-butyl [4-(3,4-dihydroquinolin-1(2H)-yl)butyl]{[(2S)-8-methyl-2,3-dihydro[1,4]dioxino[2,3-f]quinolin-2-yl]methyl}carbamate

A procedure similar to that of Step 2a, previously described, using (8-methyl-2,3-dihydro-[1,4]dioxino[2,3-f]quinolin-2-yl)-(4-oxo-butyl)-carbamic acid tert butyl ester (1c) (0.12 g, 0.31 mmol) and tetrahydroquinoline in place of 5-fluoroindoline afforded product as colorless oil.

15b: 4-(3,4-dihydroquinolin-1(2H)-yl)-N-{[(2S)-8-methyl-2,3-dihydro[1,4]dioxino[2,3-f]quinolin-2-yl]methyl}butan-1-amine

A procedure similar to Step 2b, previously described, using tert-butyl [4-(3,4-dihydroquinolin-1(2H)-yl)butyl]{[(2S)-8-methyl-2,3-dihydro[1,4]dioxino[2,3-f]quinolin-2-yl]methyl}carbamate (15a) provided the title amine as the hydrochloride salt; MS (ES) m/z=418.5 [M+H]+.

### Example 16

### 4-(5-fluoro-2,3-dihydro-1H-indol-1-yl)-N-{[(2S)-8-methyl-2,3-dihydro[1,4]dioxino[2,3-f]quinolin-2-yl]methyl}butan-1-amine

16a: Tert-butyl [4-(5-fluoro-2,3-dihydro-1H-indol-1-yl)butyl]{[(2S)-8-methyl-2,3-dihydro[1,4]dioxino[2,3-f]quinolin-2-yl]methyl}carbamate

A procedure similar to that of Step 2a, previously described, using (8-methyl-2,3-dihydro-[1,4]dioxino[2,3-f]quinolin-2-yl)-(4-oxo-butyl)-carbamic acid tert butyl ester (1c) (0.10 g, 0.3 mmol) and 6-fluoroindoline (0.14 g, 1.0 mmol) in place of 5-fluoroindoline provided the product as a colorless oil (0.090 g, 69%); MS (ES) nm/z=522.3 [M+H]+.

16b: 4-(5-fluoro-2,3-dihydro-1H-indol-1-yl)-N-{[(2S)-8-methyl-2,3-dihydro[1,4]dioxino[2,3-f]quinolin-2-yl]methyl}butan-1-amine

A procedure similar to that of Step 2b, previously described, using tert-butyl [4-(5-fluoro-2,3-dihydro-1H-indol-1-yl)butyl]{[(2S)-8-methyl-2,3-dihydro[1,4]dioxino[2,3-f]quinolin-2-yl]methyl}carbamate (16a) (0.090 g, 0.2 mmol) which provided the desired amine as the hydrochloride salt (0.051 g); MS (ES) m/z=422.2 [M+H]+.

### Example 17

### 8-OH-DPAT Binding Human 5-HT_{1A} Receptor Cell Line

Affinity for the serotonin 5-HT_{1A} receptor was established by testing each compound's ability to displace [³H]-OHDPAT (dipropylaminotetralin) from the 5-HT_{1A} serotonin receptor following a modification of the procedure of Hall et al., J. Neurochem. 44:1685-1696 (1985). Stably transfected Chinese hamster ovary (CHO) cells were plated and grown in Dulbecco's Modified Eagle's Medium (DMEM) containing 10% heat inactivated fetal bovine serum (FBS) and non-essential amino acids. Cells were scraped off the plate, transferred to centrifuge tubes, and washed twice by centrifugation (2000 rpm for 10 minutes at 4° C.) in buffer (50 mM Tris, pH 7.5). The resulting pellets were aliquoted and stored at −80° C. On the day of assay, the cells were thawed on ice, and re-suspended in buffer. The binding assay was performed in a 96-well microtiter plate in a total volume of about 250 μL. Non-specific binding was determined in the presence of 10 μM 5-HT; the final radio ligand concentration was 1.5 nM. Following a 30 minute incubation at room temperature, the reaction was terminated by the addition of ice cold buffer and rapid filtration through a GF/B filter presoaked for 30 minutes in 0.5% polyethyleneimine (PEI). Compounds were initially tested in a single point assay to determine percent inhibition at 1 μM, 0.1 μM, and 0.01 μM. Subsequently, Kᵢ values were determined for compounds defined to be active. Percent inhibition Kᵢ was determined by RFComp (Lundon Software, Chagrin Falls, Ohio), with serotonin and 8-OH-DPAT as the reference compounds. Results are presented below in Table 1. These data demonstrate the very high affinity of compounds described herein for the 5-HT_{1A} receptor, whereby lower Kᵢ values correlate with a higher affinity for the receptor.

High affinity for the receptor is evidence of a compound's ability to directly modulate activity at the 5-HT_{1A} receptor. This Example also demonstrates a method for determining the Kᵢ of a compound.

### Example 18

### ³H-Paroxetine Binding to Assess Affinity of Drugs for the Serotonin Transporter

A protocol similar to that used by Cheetham et al. (Neuropharmacol. 32:737-743, 1993) was used to determine the affinity of compounds for the serotonin transporter (i.e., by determining the relative ability of a compound to displace ³H-paroxetine from male rat frontal cortical membranes). Briefly, frontal cortical membranes prepared from male Sprague Dawley rats were incubated with ³H-paroxetine (0.1 nM) for 60 minutes at 25° C. All tubes also contained either vehicle, test compound (one to eight concentrations), or a saturating concentration of fluoxetine (10 μM) to define specific binding. All reactions were terminated by the addition of ice cold Tris buffer followed by rapid filtration using a Tom Tech filtration device to separate bound from free ³H-paroxetine. Bound radioactivity was quantitated using a Wallac 1205 Beta Plate® counter. Nonlinear regression analysis was used to determine IC₅₀ values which were converted to Kᵢ values using the method of Cheng and Prusoff (Biochem. Pharmacol. 22: 3099-3108, 1973); Kᵢ=IC₅₀/((Radioligand conc.)/(1+KD)). Results are presented in Table 1.

These data demonstrate that all compounds tested possess affinity for the 5-HT reuptake site with the majority of compounds possessing a Ki value of <100 nM. This affinity translates to the blockade of 5-HT uptake, i.e., the ability of a compound increase synaptic 5-HT levels and to act like an SSRI.

### Example 19

### cAMP Radioimmunoassay (RIA) in Human 5-HT_{1A} Receptor Cell Line

Certain effects of 5-HT (e.g., certain effects mediated by the interaction of 5-HT with the 5-HT_{1A} receptor) are mediated by cAMP. To test the ability of compounds described herein to modulate cAMP, a radioimmunoassay (RIA) method was used. Briefly, stably transfected CHO cells were grown in DMEM containing 10% heat inactivated FBS and non-essential amino acids. The cells were plated at a density of 10⁶ cells per well in a 96-well plate and incubated for 2 days in a CO₂ incubator. On the second day, the medium was replaced with 0.1 ml Krebs and incubated 15 minutes at 37° C. The medium was then replaced with Krebs buffer containing 500 μM isobutylmethylxanthine (IBMX) and incubated for approximately 5 minutes at 37° C. Wells were treated with forskolin (1 μM final concentration) followed immediately by test compound (0.1 and 1 μM for the initial screen) and incubated for an additional 10 minutes at 37° C. The reaction was terminated by removal of the medium and the addition of 0.5 ml ice cold assay buffer (supplied in the RIA kit). Plates were stored at −20° C. prior to assessment of cAMP formation by RIA. The cAMP kit was obtained from Amersham (Piscataway, N.J.) and the RIA was performed as per the kit instructions. In the initial screen, percent inhibition of forskolin stimulated cAMP was measured. Compounds in this group demonstrated high efficacy for the 5-HT_{1A} receptor and EC₅₀ concentrations only are reported. These compounds behaved as either full agonists or partial agonists. 5-HT, BMY 7378, buspirone, and 8-OH-DPAT were used as reference compounds. Results are presented Table 1.

All compounds tested inhibited the forskolin-stimulated increase in 5-HT. This indicates that the compounds are full agonists or partial agonists.

### Example 20

### Inhibition of 3H-5-HT Uptake by Cells Possessing the Human 5-HT Transporter

A human carcinoma cell line (Jar cells) possessing low endogenous levels of the 5-HT-transporter were seeded into 96 well plates and treated with staurosporine at least 18 hours prior to assay. [Staurosporine greatly increases the expression of the 5-HT-Transporter.] On the day of assay, vehicle, excess of fluoxetine, or test compound was added to various wells on the plate. ³H-5-HT was then aliquoted into all wells and the samples were incubated at 37° C. for 5 minutes. The wells were then washed with ice cold 50 mM Tris HCl (pH 7.4) buffer and aspirated to remove free ³H-5-HT. 25 μL 0.25 M NaOH was then added to each well to lyse the cells and 75 μL scintillation cocktail (Microscint™ 20) was added prior to quantitation on a Packard TopCount® machine. Tubes with vehicle represent total possible uptake, radioactivity counted in tubes with excess fluoxetine represent nonspecific binding/uptake and were subtracted from the total possible uptake to give total possible specific uptake. This nonspecific binding (usual low in number) was then subtracted from the counts obtained in wells with various test compounds (or different concentrations of a test compound) resulting in uptake in the presence of drug. Specific uptake was then expressed as a percent of control values and was analyzed using nonlinear regression analysis (Prizm) to determine IC₅₀ values. If the compound was active in inhibiting 5-HT uptake, its counts will be close to that obtained with fluoxetine. Results are presented in Table 1.

As the results demonstrate, all compounds tested demonstrated an ability to block the reuptake of ³H-5-HT. This blockade, in vivo, would be expected to result in an increase in extracellular 5-HT concentrations.

TABLE 1

| | h5HT_{1A} | | 5HT Uptake | |
|---|---|---|---|---|
| | Affinity | cAMP | r5HT-T | h5HT-T |
| | Ki (nM) | EC₅₀ (nM) | Ki (nM) | IC₅₀ (nM) |
| | (Example | (Example | (Example | (Example |
| Compound | 17) | 19) | 18) | 20) |
| Example 1 | 2.45 | 1.58 | 13 | 150 |
| Example 2 | 0.6 | 3.76 | 10.2 | n/d |
| Example 3 | 0.35 | 1.88 | 2.8 | 107 |
| Example 4 | 0.27 | 1.57 | 7.7 | 32.9 |
| Example 5 | 0.62 | 3.44 | 21.1 | 180 |
| Example 6 | 2.58 | 0.77 | 40.8 | n/d |
| Example 7 | 3.73 | 1.63 | 12.3 | 77 |
| Example 8 | 1.44 | 1.48 | 7.2 | 76.9 |
| Example 9 | 1.41 | 1.18 | 27 | n/d |
| Example 10 | 3.22 | 1.95 | 115.5 | n/d |
| Example 11 | 2.77 | 1.43 | 14.7 | 4709 |
| Example 12 | 0.79 | 3.06 | 36.2 | n/d |
| Example 13 | 0.57 | 2.49 | 3.5 | n/d |
| Example 14 | 0.19 | 1.66 | 3.3 | 161 |
| Example 15 | 0.66 | 9.25 | 11.8 | 438 |
| Example 16 | 0.45 | 31.4 | 9.6 | n/d |

n/d: not determined

### OTHER EMBODIMENTS

It is to be understood that while the invention has been described in conjunction with the detailed description thereof, the foregoing description is intended to illustrate and not limit the scope of the invention, which is defined by the scope of the appended claims. Those skilled in the art will readily appreciate that numerous changes and modifications can be made to the embodiments and that such changes and modifications can be made without departing from the spirit of the invention and will recognize, or be able to ascertain, using no more than routine experimentation, many equivalents to the specific embodiments described herein. Other aspects, advantages, and modifications are within the scope of the following claims.

We claim:

1. A compound of Formula I: 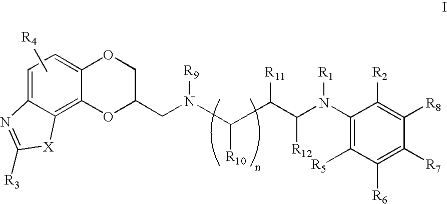
or an enantiomer, diastereomer, tautomer, pharmaceutically acceptable salt or solvate thereof, wherein
X is —O—, —N═CH—, —CR₁₃═CH—, —CR₁₃═N—, —CH═N—, —CH═CR₁₃—, —N═CR₁₃— or —NR₁₃—, in which R₁₃ is hydrogen or (C₁-C₆)-alkyl;
R₁ is hydrogen, (C₁-C₆)-alkyl, (C₁-C₆)-haloalkyl, (C₂-C₆)-alkenyl, or (C₂-C₆)-alkynyl;
R₂ is hydrogen, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₁₄, —OSO₂R₁₄, —SR₁₄, —SO₂R₁₄, —SO₂NR₁₄R₁₅, —NR₁₄R₁₅, —COR₁₄, —CO₂R₁₄, —NR₁₄CO₂R₁₅, —NR₁₄COR₁₅, —NR₁₄CONR₁₅, —NR₁₄SO₂R₁₅, or —CONR₁₄R₁₅; or
R₁ and R₂, when taken together with the atoms to which they are attached and the carbon interposed between them, form a 5- to 7-membered saturated ring, optionally containing an additional heteroatom selected from O, NR₁₄ or SOₘ;
R₃ is hydrogen, (C₁-C₆)-alkyl, halogen, or NR₁₄R₁₅;
R₄ is hydrogen, (C₁-C₆)-alkyl, halogen, —CF₃, —CN, —OR₁₄, —SO₂R₁₄, —NR₁₄SO₂R₁₅, —NR₁₄R₁₅, —COR₁₄, —CO₂R₁₄, —NR₁₄COR₁₅, or —CONR₁₄R₁₅;
R₉ is hydrogen or (C₁-C₃)-alkyl;
R₅, R₆, R₇, R₈, R₁₀, R₁₁ and R₁₂ are each independently hydrogen, halogen, (C₁-C₃)-alkyl, (C₁-C₃)-haloalkyl, (C₂-C₃)-alkenyl, or (C₂-C₃)-alkynyl, —CN, —CF₃, —NO₂, —CN, C(O)NR₁₄ or —OR₁₄;
R₁₄ and R₁₅ are each independently is hydrogen, (C₁-C₆)-alkyl, (C₁-C₆)-haloalkyl, (C₂-C₆)-alkenyl, or (C₂-C₆)-alkynyl; and
n and m are each independently 0, 1, or 2;
wherein when X is —O—, R₁ and R₂ are taken together with the atoms to which they are attached and the carbon interposed between them to form a 5- to 7-membered saturated ring, optionally containing an additional heteroatom selected from O, NR₁₄ or SOₘ.

2. The compound of claim 1, wherein X is —O—.

3. The compound of claim 1, wherein X is —CR₁₃═CH— in which R₁₃ is hydrogen.

4. The compound of claim 1, wherein R₃ is hydrogen, —NH₂, or (C₁-C₆)-alkyl.

5. The compound of claim 1, wherein R₃ is methyl.

6. The compound of claim 1, wherein R₄ is hydrogen, halogen, —CN, —CF₃, (C₁-C₆)-alkyl, or OR₁₄; and R₁₄ is hydrogen or (C₁-C₆)-alkyl.

7. The compound of claim 1, wherein R₄ is hydrogen.

8. The compound of claim 1, wherein R₁ is hydrogen or (C₁-C₆)-alkyl; R₂ is hydrogen, halogen, —CN, —CF₃, (C₁-C₆)-alkyl, or OR₁₄; and R₁₄ is hydrogen or (C₁-C₃)-alkyl.

9. The compound of claim 1, wherein R₁ and R₂ are taken together with the atoms to which they are attached and the carbon interposed between them to form a 5- to 7-membered saturated ring, optionally containing an additional heteroatom selected from O, NR₂₅ or SOₘ.

10. The compound of claim 1, wherein R₅, R₆, R₇, and R₈ are each independently hydrogen, halogen, (C₁-C₃)-alkyl, —OR₁₄, —CN, or —C(O)NR₁₄ and R₁₄ is hydrogen or (C₁-C₆)-alkyl.

11. The compound of claim 1, wherein R₅, R₆, R₇, and R₈ are each independently hydrogen or fluorine.

12. The compound of claim 1, wherein R₉ is methyl or ethyl.

13. The compound of claim 1, wherein R₉ is hydrogen.

14. The compound of claim 1, wherein R₁₀, R₁₁, and R₁₂ are each independently hydrogen, (C₁-C₃)-alkyl, or halogen.

15. The compound of claim 1, wherein R₁₀, R₁₁ and R₁₂ are hydrogen.

16. The compound of claim 1, wherein n is 1 or 2.

17. The compound of claim 1, wherein
X is —CR₁₃═CH— and R₁₃ is hydrogen or (C₁-C₃)-alkyl;
R₁ is hydrogen or (C₁-C₆)-alkyl;
R₂ is hydrogen, halogen, (C₁-C₆)-alkyl, —CN, —CF₃, or —OR₁₄, or
R₁ and R₂, when taken together with the atoms to which they are attached and the carbon interposed between them, form a 5- to 7-membered unsaturated ring, optionally containing O;
R₃ is hydrogen (C₁-C₆)-alkyl, or —NR₁₄;
R₄ is hydrogen, halogen, (C₁-C₆)-alkyl, —CN, —CF₃, or —OR₁₄;
R₅, R₆, R₇, and R₈ are each independently hydrogen, halogen, (C₁-C₃)-alkyl, —OR₁₅, —CN, or —C(O)NR₁₄;
R₉ is hydrogen or (C₁-C₃)-alkyl;
R₁₀ R₁₁ and R₁₂ are each independently hydrogen, halogen, or (C₁-C₃)-alkyl;
R₁₄ is hydrogen or (C₁-C₆)-alkyl; and
n is 1 or 2.

18. The compound of claim 1, wherein
X is —CR₁₃═CH— in which R₁₃ is hydrogen;
R₁ is hydrogen or (C₁-C₃)-alkyl;
R₂ is hydrogen, (C₁-C₃)-alkyl, or halogen; or
R₁ and R₂, when taken together with the atoms to which they are attached and the carbon interposed between them, form a 5- to 7-membered unsaturated ring, optionally containing O;
R₃ is hydrogen or —CH₃;
R₅, R₆, R₇ and R₈ are each independently hydrogen or halogen;
R₉, R₁₀, R₁₁ and R₁₂ are each independently hydrogen or (C₁-C₃)-alkyl; and
n is 1 or 2.

19. The compound of claim 1 having Formula Ib: 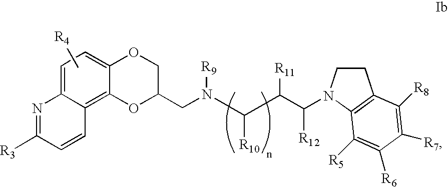
or an enantiomer, diastereomer, tautomer, pharmaceutically acceptable salt or solvate thereof.

20. The compound of claim 1 having Formula Ic: 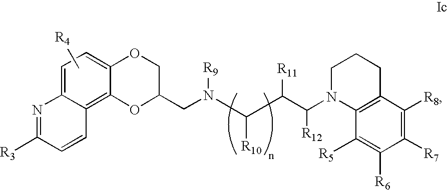
or an enantiomer, diastereomer, tautomer, pharmaceutically acceptable salt or solvate thereof.

21. The compound of claim 1 having Formula Id: 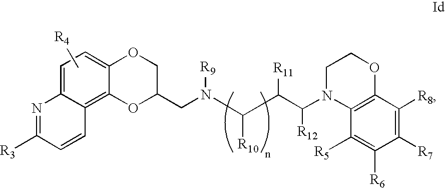
or an enantiomer, diastereomer, tautomer, pharmaceutically acceptable salt or solvate thereof.

22. A compound according to claim 1, wherein said compound is:
3-(3,4-dihydroquinolin-1(2H)-yl)-N-{[8-methyl-2,3-dihydro[1,4]dioxino[2,3-f]quinolin-2-yl]methyl}propan-1-amine;
3-(5-fluoro-2,3-dihydro-1H-indol-1-yl)-N-{[8-methyl-2,3-dihydro[1,4]dioxino[2,3-f]quinolin-2-yl]methyl}propan-1-amine;
3-(6-fluoro-2,3-dihydro-1H-indol-1-yl)-N-{[8-methyl-2,3-dihydro[1,4]dioxino[2,3-f]quinolin-2-yl]methyl}propan-1-amine;
3-(7-fluoro-3,4-dihydroquinolin-1(2H)-yl)-N-{[8-methyl-2,3-dihydro[1,4]dioxino[2,3-f]quinolin-2-yl]methyl}propan-1-amine;
3-(2,3-dihydro-1H-indol-1-yl)-N-{[8-methyl-2,3-dihydro[1,4]dioxino[2,3-f]quinolin-2-yl]methyl}propan-1-amine;
3-(2,3-dihydro-4H-1,4-benzoxazin-4-yl)-N-{[8-methyl-2,3-dihydro[1,4]dioxino[2,3-f]quinolin-2-yl]methyl}propan-1-amine;
3-(6,7-difluoro-3,4-dihydroquinolin-1(2H)-yl)-N-{[8-methyl-2,3-dihydro[1,4]dioxino[2,3-f]quinolin-2-yl]methyl}propan-1-amine;
3-(6-fluoro-3,4-dihydroquinolin-1(2H)-yl)-N-{[8-methyl-2,3-dihydro[1,4]dioxino[2,3-f]quinolin-2-yl]methyl}propan-1-amine;
3-(6,8-difluoro-3,4-dihydroquinolin-1(2H)-yl)-N-{[8-methyl-2,3-dihydro[1,4]dioxino[2,3-f]quinolin-2-yl]methyl}propan-1-amine;
3-(6-fluoro-2,3-dihydro-4H-1,4-benzoxazin-4-yl)-N-{[8-methyl-2,3-dihydro[1,4]dioxino[2,3-f]quinolin-2-yl]methyl}propan-1-amine;
N-{[8-methyl-2,3-dihydro[1,4]dioxino[2,3-f]quinolin-2-yl]methyl}-3-(2,3,4,5-tetrahydro-1H-1-benzazepin-1-yl)propan-1-amine;
N-(2-chlorophenyl)-N-methyl-N′-{[8-methyl-2,3-dihydro[1,4]dioxino[2,3-f]quinolin-2-yl]methyl}propane-1,3-diamine;
4-(6-fluoro-2,3-dihydro-1H-indol-1-yl)-N-{[8-methyl-2,3-dihydro[1,4]dioxino[2,3-f]quinolin-2-yl]methyl}butan-1-amine;
4-(3,4-dihydroquinolin-1(2H)-yl)-N-{[8-methyl-2,3-dihydro[1,4]dioxino[2,3-f]quinolin-2-yl]methyl}butan-1-amine;
4-(5-fluoro-2,3-dihydro-1H-indol-1-yl)-N-{[8-methyl-2,3-dihydro[1,4]dioxino[2,3-f]quinolin-2-yl]methyl}butan-1-amine; or
3-(3,4-dihydroquinolin-1(2H)-yl)-N-{[(2S)-8-methyl-2,3-dihydro[1,4]dioxino[2,3-f]quinolin-2-yl]methyl}propan-1-amine;
3-(5-fluoro-2,3-dihydro-1H-indol-1-yl)-N-{[(2S)-8-methyl-2,3-dihydro[1,4]dioxino[2,3-f]quinolin-2-yl]methyl}propan-1-amine;
3-(6-fluoro-2,3-dihydro-1H-indol-1-yl)-N-{[(2S)-8-methyl-2,3-dihydro[1,4]dioxino[2,3-f]quinolin-2-yl]methyl}propan-1-amine;
3-(7-fluoro-3,4-dihydroquinolin-1(2H)-yl)-N-{[(2S)-8-methyl-2,3-dihydro[1,4]dioxino[2,3-f]quinolin-2-yl]methyl}propan-1-amine;
3-(2,3-dihydro-1H-indol-1-yl)-N-{[(2S)-8-methyl-2,3-dihydro[1,4]dioxino[2,3-f]quinolin-2-yl]methyl}propan-1-amine;
3-(2,3-dihydro-4H-1,4-benzoxazin-4-yl)-N-{[(2S)-8-methyl-2,3-dihydro[1,4]dioxino[2,3-f]quinolin-2-yl]methyl}propan-1-amine;
3-(6,7-difluoro-3,4-dihydroquinolin-1(2H)-yl)-N-{[(2S)-8-methyl-2,3-dihydro[1,4]dioxino[2,3-f]quinolin-2-yl]methyl}propan-1-amine;
3-(6-fluoro-3,4-dihydroquinolin-1(2H)-yl)-N-{[(2S)-8-methyl-2,3-dihydro[1,4]dioxino[2,3-f]quinolin-2-yl]methyl}propan-1-amine;
3-(6,8-difluoro-3,4-dihydroquinolin-1(2H)-yl)-N-{[(2S)-8-methyl-2,3-dihydro[1,4]dioxino[2,3-f]quinolin-2-yl]methyl}propan-1-amine;
3-(6-fluoro-2,3-dihydro-4H-1,4-benzoxazin-4-yl)-N-{[(2S)-8-methyl-2,3-dihydro[1,4]dioxino[2,3-f]quinolin-2-yl]methyl}propan-1-amine;
N-{[(2S)-8-methyl-2,3-dihydro[1,4]dioxino[2,3-f]quinolin-2-yl]methyl}-3-(2,3,4,5-tetrahydro-1H-1-benzazepin-1-yl)propan-1-amine;
N-(2-chlorophenyl)-N-methyl-N′-{[(2S)-8-methyl-2,3-dihydro[1,4]dioxino[2,3-f]quinolin-2-yl]methyl}propane-1,3-diamine;
4-(6-fluoro-2,3-dihydro-1H-indol-1-yl)-N-{[(2S)-8-methyl-2,3-dihydro[1,4]dioxino[2,3-f]quinolin-2-yl]methyl}butan-1-amine;
4-(3,4-dihydroquinolin-1(2H)-yl)-N-{[(2S)-8-methyl-2,3-dihydro[1,4]dioxino[2,3-f]quinolin-2-yl]methyl}butan-1-amine;
4-(5-fluoro-2,3-dihydro-1H-indol-1-yl)-N-{[(2S)-8-methyl-2,3-dihydro[1,4]dioxino[2,3-f]quinolin-2-yl]methyl}butan-1-amine; or
an enantiomer, diastereomer, tautomer, pharmaceutically acceptable salt or solvate thereof.

23. A compound of Formula Ie: 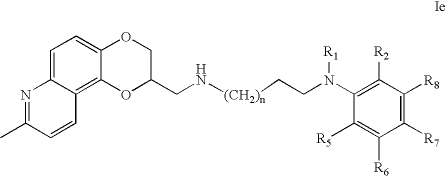
or an enantiomer, diastereomer, tautomer, pharmaceutically acceptable salt or solvate thereof, wherein
R₁ is hydrogen or (C₁-C₃)-alkyl;
R₂ is hydrogen, (C₁-C₃)-alkyl, or halogen; or
R₁ and R₂, when taken together with the atoms to which they are attached and the carbon interposed between them, form a 5- to 7-membered unsaturated ring, optionally containing O;
R₅, R₆, R₇, and R₈ are each independently hydrogen, —F or —Cl; and
n is 0, 1 or 2.

24. The compound of claim 23, wherein two of R₅, R₆, R₇, and R₈ are each independently hydrogen, —F or —Cl and the remaining R₅, R₆, R₇, or R₈ are hydrogen.

25. A pharmaceutical composition comprising
(a) a compound of claim 1 or a pharmaceutically acceptable salt thereof, and
(b) a pharmaceutically acceptable carrier.

26. A method of synthesizing a compound of Formula I: 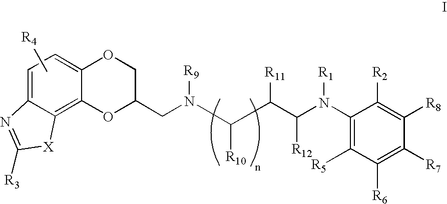
or an enantiomer, diastereomer, tautomer, pharmaceutically acceptable salt, or solvate thereof, wherein:
X is —O—, —N═CH—, —CR₁₃═CH—, —CR₁₃═N—, —CH═N—, —CH═CR₁₃—, —N═CR₁₃— or —NR₁₃—, in which R₁₃ is hydrogen or (C₁-C₆)-alkyl;
R₁ is hydrogen, (C₁-C₆)-alkyl, (C₁-C₆)-haloalkyl, (C₂-C₆)-alkenyl, or (C₂-C₆)-alkynyl;
R₂ is hydrogen, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₁₄, —OSO₂R₁₄, —SR₁₄, —SO₂R₁₄, —SO₂NR₁₄R₁₅, —NR₁₄R₁₅, —C(O), —COR₁₄, —CO₂R₁₄, —NR₁₄CO₂R₁₅, —NR₁₄COR₁₅, —NR₁₄CONR₁₅, —NR₁₄SO₂R₁₅, or —CONR₁₄R₁₅; or
R₁ and R₂, when taken together with the atoms to which they are attached and the carbon interposed between them, form a 5- to 7-membered saturated ring, optionally containing an additional heteroatom selected from O, NR₁₄ or SOₘ;
R₃ is hydrogen, (C₁-C₆)-alkyl, halogen, or NR₁₄R₁₅;
R₄ is hydrogen, (C₁-C₆)-alkyl, halogen, —CF₃, —CN, —OR₁₄, —SO₂R₁₄, —NR₁₄SO₂R₁₅, —NR₁₄R₁₅, —COR₁₄, —CO₂R₁₄, —NR₁₄COR₁₅, or —CONR₁₄R₁₅;
R₉ is hydrogen or (C₁-C₃)-alkyl;
R₅, R₆, R₇, R₈, R₁₀, R₁₁ and R₁₂ are each independently hydrogen, halogen, (C₁-C₃)-alkyl, (C₁-C₃)-haloalkyl, (C₂-C₃)-alkenyl, or (C₂-C₃)-alkynyl, —CN, —CF₃, —NO₂, —CN, —C(O)NR₁₄ or —OR₁₄;
R₁₄ and R₁₅ are each independently is hydrogen, (C₁-C₆)-alkyl, (C₁-C₆)-haloalkyl, (C₂-C₆)-alkenyl, or (C₂-C₆)-alkynyl; and
n and m are each independently 0, 1, or 2;
wherein when X is —O—, R₁ and R₂ are taken together with the atoms to which they are attached and the carbon interposed between them to form a 5- to 7-membered saturated ring, optionally containing an additional heteroatom selected from O, NR₁₄ or SOₘ;
the method comprising:
reacting a compound of Formula 1, or an enantiomer, diastereomer, tautomer, pharmaceutically acceptable salt or solvate thereof, 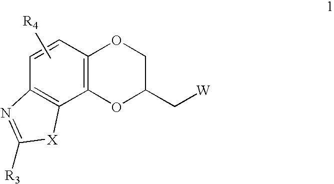
wherein X, R₃ and R₄ are as defined hereinabove, and W is a leaving group;
with an amino alcohol of Formula 2, or an enantiomer, diastereomer, tautomer, or pharmaceutically acceptable salt or solvate thereof, 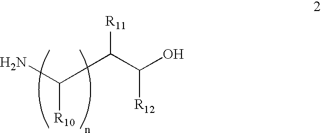
wherein R₁₀, R₁₁, R₁₂, and n are as defined hereinabove,
under conditions effective to produce a compound of Formula 3, or an enantiomer, diastereomer, tautomer, pharmaceutically acceptable salt or solvate thereof, 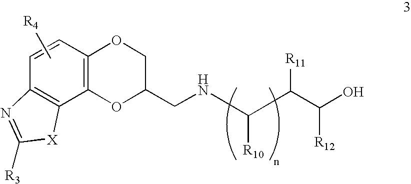
wherein X, R₃, R₄, R₁₀, R₁₁, R₁₂, and n are as defined hereinabove,
protecting the amino nitrogen of the compound of Formula 3, or an enantiomer, diastereomer, tautomer, or pharmaceutically acceptable salt or solvate thereof, under conditions effective to produce a compound of Formula 4, or an enantiomer, diastereomer, tautomer, or pharmaceutically acceptable salt or solvate thereof, 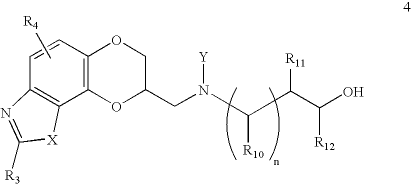
wherein X, R₃, R₄, R₁₀, R₁₁, R₁₂, and n are as defined hereinabove, and Y is an amino protecting group;
oxidizing the alcohol of the compound of formula 4, or an enantiomer, diastereomer, tautomer, or pharmaceutically acceptable salt or solvate thereof, under conditions effective to produce a compound of Formula 5, or an enantiomer, diastereomer, tautomer, or pharmaceutically acceptable salt or solvate thereof, 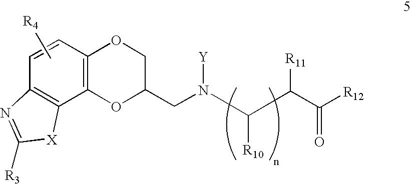
wherein X, R₃, R₄, R₁₀, R₁₁, R₁₂, Y, and n are as defined hereinabove,
reacting the compound of Formula 5, or an enantiomer, diastereomer, tautomer, or pharmaceutically acceptable salt or solvate thereof, with a compound of Formula 6, or an enantiomer, diastereomer, tautomer, pharmaceutically acceptable salt or solvate thereof, 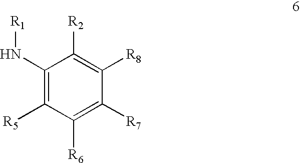
wherein R₁, R₂, R₅, R₆, R₇, and R₈ are as defined hereinabove,
under conditions effective to bring about reductive amination at the carbonyl of the compound of Formula 5, or an enantiomer, diastereomer, tautomer, or pharmaceutically acceptable salt or solvate thereof, thereby providing a compound of Formula 7, or an enantiomer, diastereomer, tautomer, pharmaceutically acceptable salt or solvate thereof, 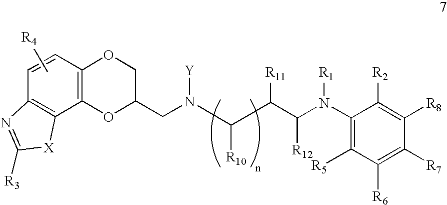
wherein X, R₁, R₂, R₃, R₄, R₅, R₆, R₇, R₈, R₁₀, R₁₁, R₁₂, Y, and n are as defined hereinabove,
reacting the compound of Formula 7, or an enantiomer, diastereomer, tautomer, pharmaceutically acceptable salt or solvate thereof, under conditions effective to remove the protecting group Y, thereby providing a compound having Formula Ia, or an enantiomer, diastereomer, tautomer, or pharmaceutically acceptable salt or solvate thereof, 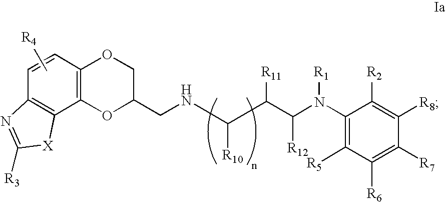
wherein X, R₁, R₂, R₃, R₄, R₅, R₆, R₇, R₈, R₁₀, R₁₁, R₁₂, and n are as defined hereinabove,
reacting the compound of Formula Ia, or an enantiomer, diastereomer, tautomer, pharmaceutically acceptable salt or solvate thereof, with a compound of Formula 8: 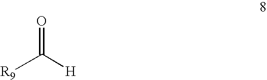
wherein R₉ is hydrogen or (C₁-C₃)-alkyl,
under conditions effective to bring about reductive amination at the nitrogen of the compound of Formula Ia, or an enantiomer, diastereomer, tautomer, pharmaceutically acceptable salt or solvate thereof, thereby providing a compound of Formula I, or an enantiomer, diastereomer, tautomer, pharmaceutically acceptable salt or solvate thereof.

27. The method of claim 26, wherein W is brosylate, tosylate, or mesylate.

28. The method of claim 26, wherein Y is t-BOC, or CBZ.

29. The method of claim 26, wherein
X is —CR₁₃═CH— and in which R₁₃ is hydrogen or (C₁-C₃)-alkyl;
R₁ is hydrogen or (C₁-C₆)-alkyl;
R₂ is hydrogen, halogen, (C₁-C₆)-alkyl, —CN, —CF₃, or —OR₁₄, or
R₁ and R₂, when taken together with the atoms to which they are attached and the carbon interposed between them, form a 5- to 7-membered unsaturated ring, optionally containing O;
R₃ is hydrogen, (C₁-C₆)-alkyl, or —NR₁₄R₁₅;
R₄ is hydrogen, halogen, (C₁-C₆)-alkyl, —CN, —CF₃, or —OR₁₄;
R₅, R₆, R₇, and R₈ are each independently hydrogen, halogen, (C₁-C₃)-alkyl, —OR₁₄, —CN, or —C(O)NR₁₄;
R₉ is hydrogen or (C₁-C₃)-alkyl;
R₁₀, R₁₁, and R₁₂ are each independently hydrogen, halogen, or (C₁-C₃)-alkyl;
R₁₄ is hydrogen or (C₁-C₆)-alkyl; and
n is 1 or 2.

30. The method of claim 26, wherein
X is —CR₁₃═CH— and in which R₁₃ is hydrogen;
R₁ is hydrogen or (C₁-C₃)-alkyl;
R₂ is hydrogen, (C₁-C₃)-alkyl, or halogen; or
R₁ and R₂, when taken together with the atoms to which they are attached and the carbon interposed between them, form a 5- to 7-membered unsaturated ring, optionally containing O;
R₃ is hydrogen or —CH₃;
R₅, R₆, R₇ and R₈ are each independently hydrogen or halogen;
R₉, R₁₀, R₁₁, and R₁₂ are each independently hydrogen or (C₁-C₃)-alkyl; and
n is 1 or 2.

31. A method of synthesizing a compound of Formula Ie, or an enantiomer, diastereomer, tautomer, or pharmaceutically acceptable salt or solvate thereof, 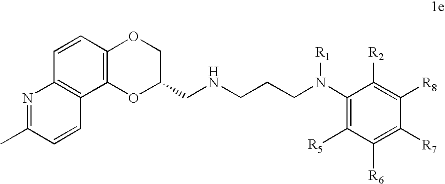
wherein:
R₁ is hydrogen, (C₁-C₆)-alkyl, (C₁-C₆)-haloalkyl, (C₂-C₆)-alkenyl, or (C₂-C₆)-alkynyl;
R₂ is hydrogen, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₁₄, —OSO₂R₁₄, —SR₁₄, —SO₂R₁₄, —SO₂NR₁₄R₁₅, —NR₁₄R₁₅, —C(O), —COR₁₄, —CO₂R₁₄, —NR₁₄CO₂R₁₅, —NR₁₄COR₁₅, —NR₁₄CONR₁₅, —NR₁₄SO₂R₁₅, or —CONR₁₄R₁₅; or
R₁ and R₂, when taken together with the atoms to which they are attached and the carbon interposed between them, form a 5- to 7-membered saturated ring, optionally containing an additional heteroatom selected from O, NR₁₄ or SOₘ;
R₅, R₆, R₇, and R₈ are each independently hydrogen, halogen, (C₁-C₃)-alkyl, (C₁-C₃)-haloalkyl, (C₂-C₃)-alkenyl, or (C₂-C₃)-alkynyl, —CN, —CF₃, —NO₂, —CN, —C(O)NR₁₄ or —OR₁₄;
R₁₄ and R₁₅ are each independently is hydrogen, (C₁-C₆)-alkyl, (C₁-C₆)-haloalkyl, (C₂-C₆)-alkenyl, or (C₂-C₆)-alkynyl; and
n and m are each independently 0, 1, or 2;
the method comprising:
reacting a compound of Formula Ie, or an enantiomer, diastereomer, tautomer, or pharmaceutically acceptable salt or solvate thereof, 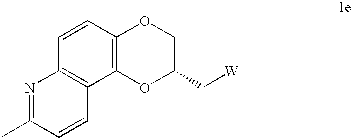
wherein W is a leaving group;
with an amino alcohol of Formula 2e, or an enantiomer, diastereomer, tautomer, or pharmaceutically acceptable salt or solvate thereof, 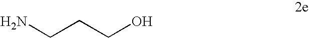
under conditions effective to produce a compound of Formula 3e, or an enantiomer, diastereomer, tautomer, or pharmaceutically acceptable salt or solvate thereof, 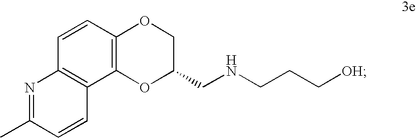
protecting the amino nitrogen of the compound of Formula 3e, or an enantiomer, diastereomer, tautomer, or pharmaceutically acceptable salt or solvate thereof, under conditions effective to produce a compound of Formula 4e, or an enantiomer, diastereomer, tautomer, or pharmaceutically acceptable salt or solvate thereof, 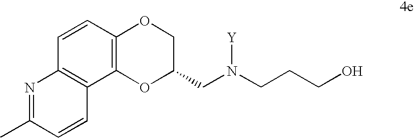
wherein Y is an amino protecting group;
oxidizing the alcohol of the compound of formula 4e, or an enantiomer, diastereomer, tautomer, or pharmaceutically acceptable salt or solvate thereof, under conditions effective to produce a compound of Formula 5e, or an enantiomer, diastereomer, tautomer, or pharmaceutically acceptable salt or solvate thereof, 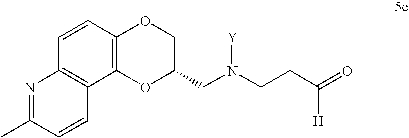
wherein Y is as defined hereinabove,
reacting the compound of Formula Se, or an enantiomer, diastereomer, tautomer, or pharmaceutically acceptable salt or solvate thereof, with a compound of Formula 6e, or an enantiomer, diastereomer, tautomer, or pharmaceutically acceptable salt or solvate thereof, 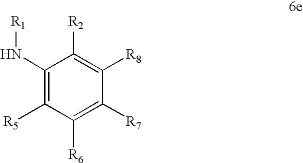
wherein R₁, R₂, R₅, R₆, R₇, and R₈ are as defined hereinabove,
under conditions effective to bring about reductive amination at the carbonyl of the compound of Formula 5, or an enantiomer, diastereomer, tautomer, or pharmaceutically acceptable salt or solvate thereof, thereby providing a compound of Formula 7e, or an enantiomer, diastereomer, tautomer, or pharmaceutically acceptable salt or solvate thereof, 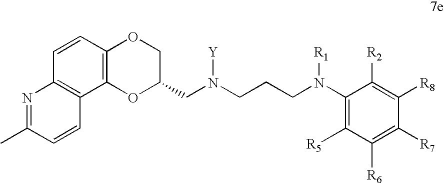
wherein R₁, R₂, R₅, R₆, R₇, R₈ and Y are as defined hereinabove,
reacting the compound of Formula 7e, or an enantiomer, diastereomer, tautomer, or pharmaceutically acceptable salt or solvate thereof, under conditions effective to remove the protecting group Y, thereby providing a compound having Formula Ie, or an enantiomer, diastereomer, tautomer, or pharmaceutically acceptable salt or solvate thereof.

32. The method of claim 31, wherein W is brosylate, tosylate, or mesylate.

33. The method of claim 31, wherein Y is t-BOC or CBZ.

34. The method of claim 31, wherein
R₁ is hydrogen or (C₁-C₆)-alkyl;
R₂ is hydrogen, halogen, (C₁-C₆)-alkyl, —CN, —CF₃, or —OR₁₄, or
R₁ and R₂, when taken together with the atoms to which they are attached and the carbon interposed between them, form a 5- to 7-membered unsaturated ring, optionally containing O;
R₅, R₆, R₇, and R₈ are each independently hydrogen, halogen, (C₁-C₃)-alkyl, —OR₁₄, —CN, or —C(O)NR₁₄; and
R₁₄ is hydrogen or (C₁-C₆)-alkyl.

35. The method of claim 31, wherein
R₁ is hydrogen;
R₂ is hydrogen, (C₁-C₃)-alkyl, or halogen; or
R₁ and R₂, when taken together with the atoms to which they are attached and the carbon interposed between them, form a 5- to 7-membered unsaturated ring, optionally containing O;
R₅, R₆, R₇ and R₈ are each independently hydrogen or halogen.

36. A method of modulating 5-HT activity in a cell, the method comprising
a. providing a cell comprising a 5-HT_{1A}; and
b. contacting the cell with a compound of claim 1 in an amount and for a time sufficient for the compound to contact the 5-HT_{1A}.

37. The method of claim 36, wherein the cell is a nervous system cell or a derivative thereof.

38. The method of claim 36, wherein a 5-HT activity in the cell is increased.

39. The method of claim 36, wherein a 5-HT activity in the cell is decreased.

40. The method of claim 36, wherein a 5-HT activity is increased and 5-HT transport is decreased.

41. A method of treating a 5-HT_{1A} related disorder in a subject in need thereof, the method comprising administering to the subject an effective amount of a compound of claim 1 or a pharmaceutically acceptable salt thereof.

42. A method of treating a central nervous system disorder in an animal in need thereof, the method comprising administering to the animal an effective amount of a compound of claim 1 or a pharmaceutically acceptable salt thereof.

43. The method of claim 42, wherein the central nervous system disorder is an anxiety-related disorder, a cognition-related disorder, depression, or schizophrenia.

44. The method of claim 43, wherein the disorder is a cognition-related disorder and the disorder is a cognitive deficit, dementia, Parkinson's disease, Huntington's disease, Alzheimer's disease, or schizophrenia.

45. The method of claim 44, wherein the cognition-related disorder is a cognitive deficient and is cognitive deficit associated with Alzheimer's disease or mild cognitive impairment.

46. The method of claim 42, wherein the disorder is an anxiety-related disorder and is attention deficit disorder, obsessive compulsive disorder, substance addiction, withdrawal from substance addiction, premenstrual dysphoric disorder, social anxiety disorder, anorexia nervosa, or bulimia nervosa.

47. A method of modulating the activity of a 5-HT_{1A} receptor, the method comprising
(a) providing a 5-HT_{1A} receptor; and
(b) contacting the receptor with at a compound of claim 1 or a pharmaceutically acceptable salt thereof in an amount and for a time sufficient to modulate 5-HT_{1A} activity.

48. The method of claim 47, wherein the 5-HT_{1A} receptor is in a subject.

49. The method of claim 47, wherein the 5-HT_{1A} receptor is in a human.

50. A method of modulating 5-HT_{1A}-mediated activity in a cell, the method comprising
(a) providing a cell comprising 5-HT_{1A};
(b) contacting the cell with a compound of claim 1 or a pharmaceutically acceptable salt thereof, wherein at least one 5-HT_{1A}-mediated activity is modulated.

51. The method of claim 50, wherein the 5-HT_{1A}-mediated activity is cyclic AMP (cAMP) level and cAMP is increased compared to a control.

52. The method of claim 50, wherein the cell is in a human.

53. A method of modulating serotonin reuptake in a cell, the method comprising
(a) providing a cell comprising a serotonin transporter;
(b) contacting the cell with a compound of claim 1 or a pharmaceutically acceptable salt thereof, wherein 5-HT reuptake is modulated.

54. The method of claim 53, wherein the cell is in a human.

55. A pharmaceutical composition for treating a 5-HT_{1A} related disorder, the composition comprising a compound of claim 1 or a pharmaceutically acceptable salt thereof.

56. A pharmaceutical composition for treating a central nervous system disorder, the composition comprising a compound of claim 1 or a pharmaceutically acceptable salt thereof.
